(19) United States
(12) Patent Application Publication
(10) Pub. No.: US 2022/0236287 A1
(43) Pub. Date: Jul. 28, 2022

(54) BIOMARKERS FOR PULMONARY EMBOLISM IN EXHALED BREATH CONDENSATE

(71) Applicants: Region Nordjylland, Aalborg University Hospital, Aalborg Ost (DK); Aarhus Universitet, Aarhus C (DK); Aalborg Universitet, Aalborg Ost (DK)

(72) Inventors: Inger Lise Gade, Aalborg (DK); Bent Honoré, Risskov (DK); Søren Risom Kristensen, Aalborg (DK)

(73) Assignees: Region Nordjylland, Aalborg University Hospital, Aalborg Øst (DK); Aarhus Universitet, Aarhus C (DK); Aalborg Universitet, Aalborg Øst (DK)

(21) Appl. No.: 17/615,693

(22) PCT Filed: Jun. 3, 2020
(86) PCT No.: PCT/EP2020/065352

§ 371 Date: Dec. 1, 2021

(30) Foreign Application Priority Data
Jun. 3, 2019 (EP) 19177903.2

(51) Int. Cl. G01N 33/68

(52) U.S. Cl. CPC G01N 33/6893; G01N 2800/226

(57) ABSTRACT

A method is provided for determining pulmonary embolism and/or increased risk thereof in a human being, comprising collecting a sample of exhalation air from said human being and determining the presence or absence in said exhaled air of one or more biomarkers associated with pulmonary embolism. Additionally, a kit that comprises the means for detecting at least one biomarker associated with pulmonary embolism or risk thereof is provided.

### TECHNICAL FIELD

The present invention relates to biomarkers used for diagnosis of pulmonary embolisms.

### BACKGROUND

Diagnosing pulmonary embolism (PE) is a big challenge. It may actually be asymptomatic, but usually the symptoms can vary from cough, chest pain, dyspnea, hemoptysis, or syncope to acute circulatory collapse and even death. The varying symptomatology, even shared with other acute cardiothoracic and respiratory diseases, can delay the very thought of pulmonary embolism as a diagnosis. The diagnostic workup includes clinical examination, D-dimer testing, arterial blood gas (A-gas) analysis, electrocardiography, echocardiography and imaging diagnostics. However, most of these tests are not specific for pulmonary embolism, and, therefore, pulmonary embolism is severely underdiagnosed.

In fact, the current diagnostic tests are faced with several issues. The biomarker D-dimer can be increased due to other conditions than pulmonary embolism and can even be falsely negative if the pulmonary embolism is sub-acute. A-gas analysis can be affected by Chronic Obstructive Pulmonary Disease (COPD) and often even large embolisms are not detectable by this analysis. Additionally, in several studies, CT scans have provided a rather poor specificity, and detectable changes on an ECG during a pulmonary embolism are not well-defined. Thus, improved diagnostic tools are needed, and there is an unmet need for a fast and non-invasive method which can diagnose pulmonary embolism with a high sensitivity.

### SUMMARY OF THE INVENTION

The provided invention has identified several significantly up- and downregulated biomarkers in exhaled breath condensate after induction of a pulmonary embolism in an animal model. Hereby will diagnosis be faster and more reliable and timely treatment can be initiated.

Several of these biomarkers have been identified as specific proteins, which have locations and/or functions that the scientific theory associates with pulmonary embolism. One such group of proteins are intracellular proteins as hypoxia and direct damage to the lung tissue due to a pulmonary embolism will cause rupture of the cell membrane. Another group of proteins are inflammatory proteins since embolisms induce an inflammatory cascade with both local and general effects on the lung parenchyma. Furthermore, pulmonary embolism will also lead to increased diffusion across the blood-air barrier because of the ischemic lung parenchymal damage, which explains why several plasma proteins were identified in the exhaled breath condensate and had a significantly altered expression.

Based on the finding of significantly up- and downregulated biomarkers substantiated by the scientific theory the present inventors has indeed established a method for detection of pulmonary embolism in an exhaled breath condensate.

### DETAILED DESCRIPTION

A central finding here relates to exhaled breath condensate for use in diagnosis of pulmonary embolism.

In one embodiment the exhaled breath condensate is subject to an assay, such that the constituents of the exhaled breath condensate can be determined. Thus, a preferred embodiment relates to the use of an assay comprising exhaled breath condensate for detection of pulmonary embolism.

An embodiment relates to an assay for detection of pulmonary embolism in a subject comprising the step of condensing exhaled breath from said subject. A similar embodiment relates to an assay for detection of pulmonary embolism in a subject, the assay comprising exhaled breath condensate from said subject.

Herein is disclosed a method for preparing an assay for the detection of pulmonary embolism in a subject by collecting a sample of exhaled breath condensate from said subject.

One embodiment relates to a method for determining pulmonary embolism and/or increased risk thereof in a human being comprising assaying a sample of exhalation air from said human being.

A superior method for detecting pulmonary embolisms are provided herein. The method is based on detection of biomarkers, which are differentially expressed in exhalation air from human beings with a pulmonary embolism or in risk thereof compared to healthy controls. In one aspect, a method is provided for determining determining pulmonary embolism and/or increased risk thereof in a human being comprising the steps of:

a. collecting a sample of exhalation air from said human being and

b. determining the presence or absence in said exhaled air of one or more biomarkers associated with pulmonary embolism or increased risk thereof.

In a preferred aspect of this method, the sample of exhalation air is an exhaled breath condensate sample.

Pulmonary embolisms can lead to blockage of the normal blood flow in the pulmonary arteries, and this blockage can cause serious problems for the circulation (worst-case a collapse) and lung function, and may lead to damage of the lungs and low oxygen levels in the blood. When an embolism obstructs a pulmonary artery, the blood is to some degree captured at the right side of the heart leading to right heart strain and possible circulatory collapse. Detection and diagnosis of a pulmonary embolism as early as possible is an important aspect of avoiding serious health effects because early treatment can then be provided. Pulmonary embolisms can be fatal if not detected and treated in due time. However, reliable detection and diagnosis is difficult. The present invention provides biomarkers, which are associated with pulmonary embolisms. More specifically, the invention provides biomarkers, which are detectable in an exhaled air sample. Thus, the biomarkers are detectable by non-invasive methods. A method is therefore provided for determining pulmonary embolism and/or increased risk thereof in a human being, where the method is based on detection of the presence or absence of specific biomarkers in exhaled air samples. In a preferred embodiment, the determination is based on a detection of level changes in one or more biomarkers.

These biomarkers allow for direct and specific diagnosis of pulmonary embolisms, which is superior to the current diagnostic tools.

Here, biomarkers applicable for a reliable diagnosis of pulmonary embolisms are presented.

A method is provided herein, which allows detection of pulmonary embolism (PE) in exhaled air. Pulmonary embolism can be a serious and acute condition. Acute pulmonary embolism is the third most common cardiovascular cause of death after acute myocardial infarction and stroke. Pulmonary embolisms occur when a blood clot breaks off from a thrombus formed in a vein in the peripheral circulation, mainly in the legs, and travels to the arteries in the lungs where it creates a blockage in the lung (pulmonary) arteries.

The symptoms of pulmonary embolisms include sudden shortness of breath, problems with breathing, chest pain, coughing with or without bloody sputum (mucus), an arrhythmia (irregular heartbeat), swelling of the leg or along a vein in the leg, pain or tenderness in the leg, increased warmth in a leg that is swollen or painful, red or discolored skin on the affected leg, feelings of anxiety or dread, bluish skin (cyanosis), lightheadedness or fainting, rapid breathing, sweating, clammy skin, increased heart rate. Subjects experiencing one or more of these symptoms are potentially affected by a pulmonary embolism and the methods provided herein are therefore preferably provided to subjects experiencing one or more of these symptoms.

A pulmonary embolism is a very serious disorder that can lead to one or several of the following conditions such as heart damage, damage to a part of the lung because of lack of blood flow to lung tissue (leading to pulmonary hypertension), low oxygen levels in the blood, damage to other organs in the body because of a lack of oxygen and/or death if the blood clot gets too large or if there are multiple blood clots.

All subjects can suffer from a pulmonary embolism. However, some subjects may have a higher risk of developing pulmonary embolisms. These subjects may have the following indications: Inactivity or immobility for long periods of time, certain inherited conditions such as blood clotting disorders or factor V Leiden, been subjected to surgery or have a broken bone (the risk is higher after surgery or an injury), have cancer, a history of cancer and/or are receiving chemotherapy and/or are bedridden or sit for long periods of time. The diagnostic methods provided herein may therefore also be provided to such subjects. In one embodiment, the detection of pulmonary embolism is the detection of increased risk thereof, i.e. increased risk of pulmonary embolism.

Additional risk factors consist of overweight or obesity, smoking, pregnancy or having given birth in the previous six weeks, taking birth control pills (oral contraceptives) or hormone replacement therapy, having diseases such as stroke, paralysis, chronic heart disease, chronic kidney disease or high blood pressure, having had recent injury or trauma to a vein, having had severe injuries, burns, or fractures of the hips or thigh bone and/or being elder or aging such as above the age of 60. Thus, the methods provided herein are therefore also relevant to provide to subjects with one or more of these risk factors.

Suspected pulmonary embolisms are detected by standard clinical tests for detection of pulmonary embolism such as clinical examination, ultrasound of the leg, computed tomography (CT) scan, lung ventilation perfusion scan, pulmonary angiography, blood tests (D-dimer testing, arterial blood gas (A-gas) analysis), echocardiography, electrocardiogram, chest X-Ray, and magnetic resonance scanning (MRI). The endogenous fibrinolytic system immediately begins to break down parts of the blood clot after coagulation. Decomposed fractions of the blood clot, i.e. Fibrin D-dimer, can be measured in the blood; however, it can take several hours before the D-dimer level increases above the normal level. Furthermore, D-dimer levels increase in a variety of other conditions such as infections and cancer. Arterial blood gas analysis is based on an arterial blood sample typically taken from the radial artery. The blood sample must be analysed within 30 minutes, and the levels of oxygen, carbon dioxide and pH, among others, is measured by electrochemical analysis in automatic blood gas analyzers.

The clinical examination concerns the examination of a subject by trained medical staff by assessment of symptoms and preferably a description by the subject of self-perceived symptoms.

Electrocardiography is a clinical diagnostic procedure in which the electrical activity of the heart is recorded over a period of time using electrodes placed over the skin of the chest wall. Every muscle contraction is caused by electronic changes. The electrodes detect these electrical changes from the heart muscle's electrophysiologic pattern of depolarizing and repolarizing during each heartbeat. An echocardiogram is a sonogram of the heart. Echocardiography uses standard two-, three-dimensional and Doppler ultrasound to create images of the heart. The echocardiogram can provide information on size and shape of the heart (internal chamber size quantification), pumping capacity, the location and extent of any tissue damage. Additionally, the echocardiogram can also give physicians estimates of the heart function, such as a calculation of the cardiac output, ejection fraction, and diastolic function (how well the heart relaxes).

Both the electro- and echocardiogram provides means for detecting symptoms of pulmonary embolism, which affect the cardiac function.

A CT pulmonary angiogram is a medical diagnostic test that employs computed tomography (CT) angiography to obtain an image of the pulmonary arteries. The patient receives an intravenous injection of an iodine-containing contrast agent. A normal CT pulmonary angiogram will show the contrast filling the pulmonary vessels, appearing as bright white. Any mass filling defects, such as an embolus, will appear dark in place of the contrast, and arteries distal to the embolus will not be contrast filled. Additionally, MRI can be used to assess pulmonary embolisms. This method is especially relevant for pregnant subjects where the usage of an iodine-containing contrast agent is contraindicated so the CT pulmonary angiogram cannot be performed.

However, the blood tests are not specific for pulmonary embolisms and the imaging tests are cumbersome. Therefore, pulmonary embolism is underdiagnosed. This is a serious problem since the treatment for pulmonary embolism is also challenging.

In order to increase the validity of the diagnosis, the methods provided herein can in certain embodiments be combined with one or more of the clinical tests such as ultrasound of the leg, computed tomography (CT) scan, lung ventilation perfusion scan, pulmonary angiography, blood tests (D-dimer testing, arterial blood gas (A-gas) analysis), echocardiography, electrocardiogram, chest X-Ray and/or MRI.

In one additional aspect, a method of treatment of a pulmonary embolism is provided, wherein the method comprises first determining a pulmonary embolism or increased risk thereof by any method disclosed herein, generally by

a. collecting a sample of exhalation air from said human being and

b. determining the presence or absence in said exhalation air of one or more biomarkers associated with pulmonary embolism or increased risk thereof, said method comprising an additional step of providing a suitable treatment for a pulmonary embolism.

Suitable treatments include anticoagulation therapy in low-risk patients and systemic or catheter guided (local) thrombolytic therapy in high-risk patients. An alternative treatment is emergency surgical embolectomy.

The sample of exhalation air can be collected by any method available in the art. The method should allow retention of relevant biomarkers present in the expiration air, which can be indicative of pulmonary embolisms. In one preferred embodiment, the sample of exhalation air is an exhaled breath condensate sample. Exhaled breath condensates can be collected by allowing the human subject to breathe into a collection device. Thus, whenever the term exhailed breath condensate is used, it implies samples of exhailed air.

Typically, the subject exhales into a collection device, which immediately cools and condensates the air, thereby providing an exhaled breath condensate. The collection device may for example comprise a cooled tube.

Thus, one embodiment of the invention relates to the use of an exhailed air collection device for diagnosis of pulmonary embolism.

In one embodiment, the exhaled air sample is obtained from a subject that is connected to a medical ventilator, where the ventilator may control the subject's inhalation and expiration. The subject may be unconscious when connected to the medical ventilator.

In another embodiment, the exhaled air sample is obtained from a subject voluntarily exhaling into a medical ventilator.

In one embodiment, a medical ventilator can be used for retaining and/or collecting the biomarkers of the provided invention, wherein a tube and preferably an associated cooling sleeve are inserted between the expiratory limb of a Y-connector and the expiratory limb of the mechanical ventilator.

In another embodiment, a medical ventilator can be used for retaining and/or collecting the biomarkers of the provided invention, wherein a tube and preferably an associated cooling sleeve are inserted directly at the expiratory limb on the medical ventilator.

In one embodiment, the exhaled air sample can be cooled and the resulting exhaled air sample can be collected in collection tube and/or strip precooled to a temperature such as below 10° C., such as below 5° C., such as below 0° C., such as below −5° C., such as below −10° C., such as below −20° C., such as below −40° C., such as below −60° C., such as below −80° C.

In one embodiment the time used for air sample collection is any given time, such as at least 1 minute, such as at least 2 minutes, such as at least 3 minutes, such as at least 4 minutes, such as at least 5 minutes, such as at least 6 minutes, such as at least 15 minutes, such as at least 18 minutes, such as at least 20 minutes, such as at least 30 minutes, such as at least 1 hour, such as at least 2 hours, such as at least 3 hours.

The collection device can comprise a filter, which is capable of removing unwanted molecules of a certain size. The breath condensate is preferably cooled immediately in the collection device in order to obtain a condensate.

The samples of expiration air can comprise relevant biomarkers, which are indicative of a pulmonary embolism. Thus, the method provided herein comprises detecting one or more biomarkers associated with pulmonary embolism or increased risk thereof. The relevant biomarkers are specified herein below.

Detection of the relevant biomarkers includes determination and/or quantification of the relevant biomarkers. Methods for detecting biomarkers are well-known and a large amount of methodologies are available to those of skill in the art.

In one embodiment, one or more biomarkers are determined by antibody-based detection assay such as an ELISA, western blot, and/or protein immunoprecipitation preferably using the samples obtained in said collection tube.

In another embodiment, the biomarkers are detected by methods selected from the group consisting of mass spectrometry, liquid chromatography-tandem mass spectrometry (LC-MS/MS), high-performance liquid chromatography (HPLC) preferably using the samples obtained in said collection tube.

In one embodiment, the biomarkers can be identified in a database, such as Uniprot, and/or quantified using label free quantification (LFQ).

In another embodiment, the biomarkers can be identified using mass spectrometry.

In one embodiment, the biomarkers can be identified by metabolomics using LC-MS/MS and/or Nuclear Magnetic Resonance (NMR). In one embodiment, the biomarkers can be identified using proteomics using LC-MS/MS and/or Nuclear Magnetic Resonance (NMR).

Preferably the assay, methods and kits provided herein further comprises the detection of one or more biomarkers. The methods and kits provided herein are based on the detection of or absence of detection of one or more biomarkers, which are associated with pulmonary embolism or risk thereof.

Generally, a biomarker is a measurable indicator of a biological state and/or condition and is by definition objective and quantifiable. The expression of biomarker(s) can indicate a risk, presence or progression of a disease, indicate the severity/prognosis and/or the susceptibility of the disease to a given treatment. In general, a biomarker can be any given molecule, including proteins, hormones, cytokines, chemokines, nucleotides, amino acids, lipids and/or metabolic intermediate products and/or by-products. The biomarkers, for which the presence or absence is determined in the methods and kits provided herein, can be proteins, metabolic intermediate products and/or by-products.

In one embodiment, the determination of one or more biomarkers is the absence or precense of such one or more biomarkes. In another embodiment, the absence or presence of such one or more biomarkes is graded and resolution enhanced to detect the levels of such biomarkers.

According to some embodiments, the method and the assay comprises determining the presence or absence of at least 1, such as at least 2, such as at least 3, 4, 5, 6, 7, 8, 9, such as at least 10, such as at least 11, 12, 13, 14, 15, 16, 17, 18, 19, such as at least 20 biomarkers associated with pulmonary embolism or risk thereof. In a preferred embodiment, the method comprises determining the presence or absence of 1-10 biomarkers, such as 1-5 biomarkers.

The link between the up- and downregulation of proteins in the exhaled breath condensate establishes an assay for detection of pulmonary embolism and/or increased risk thereof in a subject comprising the steps of:

a. collecting a sample of exhaled breath condensate from said subject; and

b. determining in said exhaled breath condensate one or more biomarkers.

In one embodiment the one or more biomarkers are proteins. That is, at least one of the biomarkers is a protein. The term protein in this context includes all peptides or polypeptides present in the human organism, including protein fragments. The proteins can contain posttranslational modifications, such as acylation, acetylation, methylation, amidation, biotinylation, formylation, phosphorylation, glutamylation, glycosylation, glycinylation, hydroxylation, iodination, isoprenylation, myristoylation, farnesylation, geranylgeranylation, oxidation, palmitoylation, polysialylation and/or sulfurylation. The proteins can be stabilized by disulphide bridges. The proteins may be detected either in their native state or as denatured proteins.

Protein biomarkers are detected by any method for identifying and/or quantifying polypeptides in a sample. In one preferred embodiment, protein biomarkers are preferably detected in a sample present in a collection tube.

In one embodiment the biomarker is one or more proteins selected from the group of proteins showing a p-value below or equal to 0.10 in FIGS. 15, 16 and 17.

In another embodiment the biomarker is one or more proteins selected from the group of proteins showing a p-value below or equal to 0.05 in FIGS. 15, 16 and 17.

In a further embodiment the biomarker is one or more proteins selected from the group of proteins showing a p-value below or equal to 0.01 in FIGS. 15,16 and 17.

In one embodiment the biomarker is one or more proteins selected from the group of proteins showing at least a 10-fold change in expression levels in FIGS. 15, 16 and 17.

In another embodiment the biomarker is one or more proteins selected from the group of proteins showing at least a 5-fold change in expression levels in FIGS. 15, 16 and 17.

In one embodiment, the biomarker is a polypeptide (designated by its gene name) selected from the group consisting of TPM3, DSTN, S100A11, ARG1, APOD, LYZ, AMY1A, ALB, BLMH, DSG1, SERPINB12, CSTA, GGCT, KATNAL2, LCN2, SERPINB4, ATMIN, IGHG1, TPI1, RPLP2, CALML3, CALML5, ALDOA, PPIB, C3, HSPA5, ARHGDIA, TKT, AKR1B10, PABPC1, ACTN4, ACTG1, HSP90AA1, TUBB4B, A2ML1, VCP, EEF1A1P5, TUBAIB, IGHG2, IGKC, IL36G, MDH1, ERP29, TYMP, LGALS7 SFN, ENO1, GSTP1, PPIA, YWHAZ and/or HIST2H2AC.

In one embodiment, the biomarker is a polypeptide (designated by its gene name) selected from the group consisting of LYZ, AMY1A, ALB, BLMH, DSG1, SERPINB12, CSTA, GGCT, KATNAL2, LCN2, SERPINB4, ATMIN, IGHG1, TPI1, RPLP2, CALML3, CALML5, ALDOA, PPIB, C3, HSPA5, ARHGDIA, TKT, AKR1B10, PABPC1, ACTN4, ACTG1, HSP90AA1, TUBB4B, A2ML1, VCP, EEF1A1P5, TUBAIB, IGHG2, IGKC, IL36G, MDH1, ERP29, TYMP, LGALS7, SFN, ENO1, GSTP1, PPIA, YWHAZ and/or HIST2H2AC.

In one embodiment, the biomarker is a polypeptide (designated by its gene name) selected from the group consisting of Tropomyosin alpha-3 (TPM3), destrin (DSTN), protein S100-A11 (S100A11), arginase 1 (ARG1) and apolipoprotein D (APOD).

The biomarkers can be either positively or negatively correlated with pulmonary embolism. The presence of some biomarkers is increased whereas other biomarkers are decreased in pulmonary embolism compared to the corresponding level in a control sample.

In one embodiment, the one or more biomarkers are positively correlated to pulmonary embolism. Preferred examples are set out in table 1.

The criteria for selection as “positive marker” for pulmonary embolism, (i.e. a protein that would be present in higher amounts in case of pulmonary embolism) are e.g. proteins significantly upregulated in the Early Post PE or Late Post PE samples compared with the Pre PE samples and not upregulated in Early Post C or late Post C compared with Pre C.

Positive markers also include proteins significantly upregulated in Early Post PE compared with Early Post C and not upregulated in Early Post C compared with Pre C.

Positive markers also include proteins significantly upregulated in Late Post PE compared with Late Post C and not upregulated in Late Post C compared with Pre C.

The term upregulated refer to an increase in the concentration and/or amount of biomarkers in a PE sample compared to a control sample.

In one embodiment, the one or more biomarkers is one or more negatively correlated biomarkers selected from the group of polypeptides set out in table 2.

The criteria for selection as “negative marker” for pulmonary embolism (i.e. a protein that would be present at very low amounts (if present at all) in case of pulmonary embolism and possibly present in higher amounts in non-PE cases) are e.g. proteins significantly downregulated in the Early Post PE or Late Post PE samples compared with Pre PE and not significantly downregulated in the Early Post C or Late Post C compared with Pre C).

Negative markers also include proteins significantly downregulated in Early Post PE compared with Early Post C and not downregulated in Early Post C compared with Pre C. Negative markers also include proteins significantly downregulated in Late Post PE compared with Late Post C and not downregulated in Late Post C compared with Pre C.

The term downregulated refers to a decrease in the concentration and/or amount of biomarkers in a PE sample compared to a control sample.

A particularly preferred assay includes detection of a combination of two or more biomarkers from table 1 and table 2. That is, the detection of high amounts of some biomarkers as well as low amounts of other biomarkers. Hereby is a sensitive assay obtained.

TABLE 1
Total group of positive markers for PE

| Protein name | Gene name |
|---|---|
| IgGFc-binding protein | FCGBP |
| Clusterin | CLU |
| Polymeric immunoglobulin receptor | PIGR |
| Apolipoprotein D | APOD |
| Serotransferrin | TF |
| Hemopexin | HPX |
| Hemoglobin subunit beta | HBB |
| Serum albumin | ALB |
| Alpha-amylase 1 | AMY1A |
| Fibronectin | FN1 |
| Lysozyme C | LYZ |
| Proteasome subunit alpha type-3 | PSMA3 |
| Mucin-5B | MUC5B |
| Lipocalin-1 | LCN1 |
| Bleomycin hydrolase | BLMH |
| Protein-glutamine gamma-glutamyltransferase 5 | TGM5 |
| PITH domain-containing protein 1 | PITHD1 |
| Cystatin-A | CSTA |
| Serpin B12 | SERPINB12 |
| Serpin A12 | SERPINA12 |
| Desmoglein-1 | DSG1 |
| Coatomer subunit beta | COPB2 |
| Apolipoprotein A-1 | APOA1 |
| Lactotransferrin | LTF |
| Target of Myb protein 1 | TOM1 |
| Cell division control protein 42 homolog | CDC42 |
| N-acetyl-D-glucosamine kinase | NAGK |
| Glucose-6-phosphate 1-dehydrogenase | G6PD |
| Heme oxygenase 2 | HMOX2 |
| Actin, cytoplasmic 1 | ACTB |
| Adenosine kinase | ADK |
| ADP/ATP translocase 3 | SLC25A6 |
| Vinculin | VCL |
| Heterogeneous nuclear ribonucleoprotein K | HNRNPK |
| Perilipin-3 | PLIN3 |
| Desmocollin-3 | DSC3 |
| Calpain-1 catalytic subunit | CAPN1 |
| Rho GTPase-activating protein 1 | ARHGAP1 |
| Zinc-alpha-2-glycoprotein | AZGP1 |
| Protein-glutamine gamma-glutamyltransferase K | TGM1 |
| Lysosome-associated membrane glycoprotein 2 | LAMP2 |
| Elongation factor 1-gamma | EEF1G |
| Peroxiredoxin-1 | PRDX1 |
| Keratin, type II cytoskeletal 2 epidermal | KRT2 |
| Gamma-glutamylcyclotransferase | GGCT |
| Annexin A4 | ANXA4 |
| Corneodesmosin | CDSN |
| Hemoglobin subunit alpha | HBA |
| 40S ribosomal protein S25 | RPS25 |
| Kynureninase | KYNU |
| Submaxillary gland androgen-regulated protein 3B | SMR3B |
| Creatine kinase B-type | CKB |
| 26S proteasome non-ATPase regulatory subunit 11 | PSMD11 |
| Ubiquitin-like protein 3 | UBL3 |
| Haptoglobin | HP |
| Annexin A5 | ANXA5 |
| Proteasome subunit beta type-4 | PSMB4 |
| Clathrin heavy chain 1 | CLTC |
| Ig kappa chain V-III region POM | IGKV3D-7 |
| Alpha-1-antitrypsin | SERPINA1 |

TABLE 2
Total group of negative markers for PE

| Protein name | Gene name |
|---|---|
| 60S ribosomal protein L11 | RPL11 |
| Polyadenylate-binding protein 1 | PABPC1 |
| Transitional endoplasmic reticulum ATPase | VCP |
| Thymidine phosphorylase | TYMP |
| Katanin p60 ATPase-containing subunit A-like 2 | KATNAL2 |
| Tubulin alpha-1B chain | TUBA1B |
| Alpha-2-macroglobulin-like protein 1 | A2ML1 |
| Epiplakin | EPPK1 |
| Neutrophil gelatinase-associated lipocalin | LCN2 |
| Serpin B4 | SERPINB4 |
| ATM interactor | ATMIN |
| Farnesyl pyrophosphate synthase | FDPS |
| Fructose-bisphosphate aldolase A | ALDOA |
| Tubulin beta-4B chain | TUBB4B |
| Myosin-9 | MYH9 |
| Putative elongation factor 1-alpha-like 3 | EEF1A1P5 |
| Peptidyl-prolyl cis-trans isomerase B | PPIB |
| T-complex protein 1 subunit theta | CCT8 |
| Alpha-actinin-4 | ACTN4 |
| Elongation factor 2 | EEF2 |
| 60S ribosomal protein L23a | RPL23A |
| Ig gamma-1 chain C region | IGHG1 |
| Galectin-7 | LGALS7 |
| 14-3-3 protein sigma | SFN |
| Ganglioside GM2 activator | GM2A |
| Alpha-enolase | ENO1 |
| Plakophilin-1 | PKP1 |
| Glutathione S-transferase P | GSTP1 |
| Triosephosphate isomerase | TPI1 |
| Aldo-keto reductase family 1 member B10 | AKR1B10 |
| Complement C3 | C3 |
| Actin, cytoplasmic 2 | ACTG1 |
| Phosphoglycerate kinase 1 | PGK1 |
| Serpin B5 | SERPINB5 |
| Serpin B3 | SERPINB3 |
| Desmoplakin | DSP |
| Transketolase | TKT |
| Heat shock protein HSP 90-alpha | HSP90AA1 |
| 60S acidic ribosomal protein P2 | RPLP2 |
| Calmodulin-like protein 3 | CALML3 |
| Cofilin-1 | CFL1 |
| Calmodulin-like protein 5 | CALML5 |
| Heat shock protein beta-1 | HSPB1 |
| Rho GDP-dissociation inhibitor 1 | ARHGDIA |
| Polypyrimidine tract-binding protein 1 | PTBP1 |
| 78 kDa glucose-regulated protein | HSPA5 |
| Glucosylceramidase | GBA |
| 26S proteasome non-ATPase regulatory subunit 6 | PSMD6 |
| 10 kDa heat shock protein, mitochondrial | HSPE1 |
| Alpha-2-HS-glycoprotein | AHSG |
| Signal transducer and activator of transcription 3 | STAT3 |
| Cellular retinoic acid-binding protein 2 | CRABP2 |
| Ig gamma-2 chain C region | IGHG2 |
| Inter-alpha-trypsin inhibitor heavy chain H4 | ITIH4 |
| Small nuclear ribonucleoprotein Sm D3 | SNRPD3 |
| Ig lambda-3 chain C regions | IGLC3 |
| Ig kappa chain C region | IGKC |
| lnterleukin-36 gamma | IL36G |
| Interferon alpha-inducible protein 27, mitochondrial | IFI27 |
| Plastin-3 | PLS3 |
| ATP-dependent RNA helicase A | DHX9 |
| Mitogen-activated protein kinase kinase kinase kinase 4 | MAP4K4 |
| Endoplasmic reticulum resident protein 29 | ERP29 |
| Ubiquitin-60S ribosomal protein L40 | UBA52 |
| Proteasome activator complex subunit 2 | PSME2 |
| Histone H1.4 | HIST1H1E |
| Gelsolin | GSN |
| ATP synthase subunit beta, mitochondrial | ATP5B |
| 40S ribosomal protein S7 | RPS7 |
| Histone H2A type 2-C | HIST2H2AC |
| Histone H1.5 | HIST1H1B |
| Protein disulfide-isomerase | P4HB |
| Protein POF1B | POF1B |
| 60S ribosomal protein L22 | RPL22 |
| F-actin-capping protein subunit alpha-1 | CAPZA1 |
| 40S ribosomal protein S4, X isoform | RPS4X |
| Peptidyl-prolyl cis-trans isomerase A | PPIA |
| Ezrin | EZR |
| Plakophilin-3 | PKP3 |
| 14-3-3 protein zeta/delta | YWHAZ |
| ADP-ribosylation factor 5 | ARF5 |
| Iron/zinc purple acid phosphatase-like protein | PAPL |
| Rab GDP dissociation inhibitor beta | GDI2 |
| Nucleophosmin | NPM1 |
| Nascent polypeptide-associated complex subunit alpha | NACA |
| Ras-related protein Rab-7a | RAB7A |
| THO complex subunit 4 | ALYREF |
| Protein S100-A11 | S100A11 |
| V-type proton ATPase catalytic subunit A | ATP6V1A |
| Protein disulfide-isomerase A3 | PDIA3 |
| Cathepsin D | CTSD |
| Proteasome subunit alpha type-6 | PSMA6 |
| Actin-related protein 3 | ACTR3 |
| Purine nucleoside phosphorylase | PNP |
| Peroxiredoxin-2 | PRDX2 |
| Protein S100-A7 | S100A7 |
| Adenylyl cyclase-associated protein 1 | CAP1 |
| Histone H4 | HIST1H4A |
| Protein S100-A8 | S100A8 |
| Drebrin-like protein | DBNL |
| Acetyl-CoA acetyltransferase, cytosolic | ACAT2 |
| Proteasome subunit alpha type-2 | PSMA2 |
| Creatine kinase U-type, mitochondrial | CKMT1A |
| Heat shock cognate 71 kDa protein | HSPA8 |
| Histone H2B type 1-M | HIST1H2BM |
| Acyl-CoA-binding protein | DBI |
| ATP synthase subunit alpha, mitochondrial | ATP5A1 |
| Three prime repair exonuclease 2 | TREX2 |
| Filamin-B | FLNB |
| Junction plakoglobin | JUP |

Without being bound by the presence theory, the inventors recognize that pulmonary embolism can lead to a release of intracellular proteins due to hypoxia and direct damage on the lung tissue. These intracellular proteins include proteins localized to the cytoplasm, intracellular membrane-bounded organelles, intracellular non-membrane-bounded organelles or to the cytosol. Generally, hypoxia and tissue damage will lead to increased levels of these intracellular proteins in the exhaled breath condensate. We speculate, that the intracellular proteins escape the cell and can be found in the exhailed breath. However, due to the tissue damage, enzymatic degradation of proteins outside of cellular protection will take place, resulting in a mared decrease of certain other biomarkers.

Any intracellular proteins provided in the present invention were identified using a STRING analysis (including bioinformatics characterization) (FIG. 40). The degree of interaction between the proteins in the analysis is significantly higher than expected for a random set of proteins (p<1×10-16). Out of the proteins, 122 were found to be associated with the cytoplasm, 76 with the cytosol, 91 with intracellular membrane-bounded organelles and 49 with intracellular non-membrane-bounded organelles.

Thus, in one embodiment, the biomarkers of the present invention are intracellular proteins. In another embodiment, the biomarkers are the intracellular proteins provided in FIG. 41.

In a further embodiment, the intracellular biomarkers are one or more proteins (designated by its gene name) selected from a group comprising the following proteins: TF, ALB, LYZ, BLMH, APOA1, LTF, HBB, HBA1, HBA2, TUBA1B, LCN2, ALDOA, RPLP2 and CLAML5.

In one embodiment, the biomarkers of the present invention are major plasma proteins and proteins associated with hemopthysis.

In another embodiment, the biomarkers are the major plasma proteins and proteins associated with hemopthysis provided in FIG. 42.

In one embodiment, the biomarkers are one or more major plasma proteins (designated by its gene name) selected from a group comprising the following proteins: SERPINA1, C3, HP, TF, FN1, LTF, SERPINC1, PLG, A2M, FGA, FGB, FGG, KLKB, serpins superfamily, Immunoglobulin superfamily, ORM1 and/or ORM2. In one similar embodiment, one of the biomarkers is albumin (ALB).

In one embodiment, the one or more biomarkers are one or more proteins associated with coagulation.

In another embodiment, the biomarkers are one or more proteins associated with coagulation (designated by its gene name) selected from a group comprising the following proteins: CDCl₄2, ANXA5, SERPINA1, CLU, C3, SERPINC1 and/or SERPIN B2.

Without being bound by the presence theory, the inventors recognize that pulmonary embolism can lead to ischemic pulmonary parenchymal necrosis and thus sometimes macroscopic hemoptysis. Sufficient impedance of these sources can cause infarction and subsequent tissue necrosis. Inflammatory mediators from ischemic lung parenchyma limit gas exchange following vasoconstriction and bronchoconstriction.

When ischemia of lung tissue is not reversed infarction ensues. Consequently, “micro-hemoptysis” should be measurable in the exhaled breath condensate as a sign of the parenchymal necrosis in pulmonary embolism. Additionally, pulmonary embolism will lead to increased diffusion across the blood-air barrier because of the ischemic lung parenchymal damage. This will lead to an abundance of plasma proteins in the exhaled breath condensate.

Thus, in one embodiment, the biomarkers of the present invention are major plasma proteins and proteins associated with hemoptysis.

In another embodiment, the biomarkers are the major plasma proteins and proteins associated with hemoptysis provided in FIG. 42.

In a further embodiment, the major plasma proteins and proteins associated with hemoptysis biomarkers are one or more proteins (designated by its gene name) selected from a group consisting of the following proteins: ALB, HBB, HBA1, HBA2, HMOX, HPX, HP and TF.

In one embodiment, the biomarkers are one or more major plasma proteins (designated by its gene name) selected from a group comprising the following proteins: SERPINA1, C3, HP, TF, FN1, LTF, SERPINC1, PLG, A2M, FGA, FGB, FGG, KLKB, serpins superfamily, Immunoglobulin superfamily, ORM1 and ORM2.

In a second embodiment, the biomarkers are one or more proteins associated with coagulation (designated by its gene name) selected from a group comprising the following proteins: CDCl₄2, ANXA5, SERPINA1, CLU, C3, SERPINC1 and SERPIN B2.

In a third embodiment, the biomarkers are one or more proteins associated with hemostasis, heme metabolism and/or a hemoglobin scavenger (designated by its gene name) selected from a group comprising the following proteins: HMOX, HPX, HP, ALB, APOA1, VCL, ACTB, CFL1 and TUBA1B.

Without being bound by the presence theory, the inventors recognize that pulmonary embolism can lead to postembolic pulmonary inflammation. Thrombosis induces an inflammatory cascade with both local and general effects on the lung parenchyma. It has been shown, that pulmonary thrombosis leads to the recruitment of neutrophils within 15 minutes after thrombus formation. The neutrophils possibly accumulate in the alveolar fluid. Also, common inflammatory cytokines such as thrombin, arachidonic-acid-derived factors, platelet activating factor, leukotriene B4, tromboxane A2, interleukin 6, vascular endothelial growth factor could be present in the alveolar fluid enabling a detection in the exhaled breath condensate.

Any inflammatory proteins provided in the present invention were identified using a STRING analysis where proteins associated with neutrophil degranulation, platelet degranulation and acute inflammatory response are depicted in FIG. 43 and proteins associated with neutrophil degranulation, platelet degranulation and the innate immune system are depicted in FIG. 44.

Thus, in one embodiment, the biomarkers of the present invention are inflammatory proteins.

In another embodiment, the biomarkers are one or more inflammatory proteins provided in FIG. 45.

In a further embodiment, the inflammatory proteins are one or more proteins (designated by its gene name) selected from a group comprising the following proteins: ALB, HBB, LTF, HP, LYZ, LCN2, FN1 and SERPINC1.

Based on additional statistics using student's t-test without further correction for multiple comparisons in order not to increase the risk of overlooking important markers, additional relevant proteins were identified.

Thus, in one embodiment, the biomarkers are one or more proteins provided in FIG. 46.

In a further embodiment, the proteins are one or more proteins (designated by its gene name) selected from a group comprising the following proteins: ACTB, AMY1A, ALB, BLMH, CSTA, HBB, LCN1, LYZ and TF.

Additionally, favourite negative biomarkers based on dataquality, fold-changes, p-values and literature findings were identified.

Thus, in one embodiment, the biomarkers are one or more proteins provided in FIG. 47.

In a further embodiment, the proteins are one or more proteins (designated by its gene name) selected from a group comprising the following proteins: IL36G, LCN2, TYMP, RPLP2 and ALDOA.

Using a prediction model based on penalized regression, additional important biomarkers for pulmonary embolism were identified. During estimation, the prediction model was “punished” when using too many parameters. Therefore, the model aimed to obtain the best balance between a good performance (how good the model was at identifying pulmonary embolism) and how many proteins the model used. FIG. 48A indicates that the optimal model obtained for comparison of Pre PE vs. Early post PE has a misclassification error of ˜0.5%. The optimal model (based on 57 proteins, FIG. 49A) classified 28 out of 28 samples correctly. FIG. 48B indicates that the optimal model for comparison of Pre PE vs. Late post PE has a misclassification error of ˜2.5%. The optimal model (based on 5 proteins, FIG. 49B) classified 24 out of 27 samples correctly.

Thus, in one embodiment, the biomarkers are one or more proteins identified in the prediction model provided in FIG. 49.

In a second embodiment the biomarkers are one or more proteins identified in the prediction model provided in FIG. 49A.

In a third embodiment the biomarkers are one or more proteins identified in the prediction model provided in FIG. 49B.

Additional statistics using a student's t-test, paired and unpaired without further correction for multiple comparisons in order not to increase the risk of overlooking important markers, identified proteins that were up-regulated in more than one comparison in the dataset.

Thus, in one embodiment, the biomarkers are one or more proteins provided in FIG. 50.

In a further embodiment, the proteins are one or more proteins (designated by its gene name) selected from a group comprising the following proteins: ALB, APOA1, BLMH, and LTF.

The designated biomarkers can be used for detection of a pulmonary embolism, such as acute pulmonary embolism. In another embodiment, the biomarkers can be used for detection of an increased risk of pulmonary embolism.

The biomarkers provided herein can be used for determining pulmonary embolism at any time after the pulmonary embolism has occurred.

Early detection is relevant for the acute setting where the necessity for a fast diagnosis is vital. Late detection can be relevant for all patients not initially diagnosed with pulmonary embolism or who is provided with late medical care. Generally, the provided method allows detection of pulmonary embolism at any given time after onset.

In one embodiment, the biomarkers can detect PE after onset within 0-48 hours, such as 0-36 hours, such as 0-24 hours, such as 0-18 hours, such as 0-12 hours, such as 0-8 hours, such as 0-4 hours, such as 0-2.5 hours and/or such as 0-1 hour.

In another embodiment the biomarkers can detect PE late after onset within 1.5-48 hours, such as 1.5-24 hours, such as 1.5-12 hours, such as 90-600 minutes, such as 90-360, such as 90-210 minutes, such as 120-180 minutes and/or such as 135-165 minutes after onset.

In yet another embodiment, the provided method allows detection of PE early after onset within 0-90 minutes, such as 0-60 minutes, such as 10-50 minutes and/or such as 15-45 minutes after onset.

One aspect also relates to a kit, which comprises means for detecting at least one biomarker associated with pulmonary embolism or risk thereof.

### DESCRIPTION OF DRAWINGS

FIG. 1.

Overview of the study population in the validation study. EBC: Exhaled Breath Condensate. PE: Pulmonary Embolism.

FIG. 2.

Collection times for the Exhaled Breath Condensates (EBCs) for the PE animals, negative controls and for the condensate from the mechanical ventilator (validation study). Pre PE means sample obtained prior to pulmonary embolism (PE), Post PE means sample obtained early after PE and Late Post PE means samples obtained at a later timepoint after PE.

FIG. 3.

Protein amount (μg) per 100 liter of exhaled air according to collection time and animal group and for the mechanical ventilator (MV) (validation study). Pre PE means sample obtained prior to pulmonary embolism (PE), Post PE means sample obtained early after PE and Late Post PE means samples obtained at a later timepoint after PE.

FIG. 4.

Protein concentration in the EBC according to collection time and animal group and in the condensate from the mechanical ventilator (validation study). Pre PE means sample obtained prior to pulmonary embolism (PE), Post PE means sample obtained early after PE and Late Post PE means samples obtained at a later timepoint after PE. C refers to the control samples obtained at identical time points in control animals.

FIG. 5.

Overview of number of proteins significantly upregulated (upper circles) and downregulated (lower circles) in Early Post PE and Late Post PE compared with Pre PE (validation study, unpaired analysis). Pre PE means sample obtained prior to pulmonary embolism (PE), Post PE means sample obtained early after PE and Late Post PE means samples obtained at a later timepoint after PE.

FIG. 6.

Overview of number of proteins significantly upregulated (upper circle) and downregulated (lower circle) in Early Post PE compared with Early Post C (validation study, unpaired analysis combined). Post PE means sample obtained early after PE and Post C means control sample obtained at same time point as the Post PE sample.

FIG. 7.

Overview of number of proteins significantly upregulated (upper circles) and downregulated (lower circle) in Late Post PE compared with Late Post C (validation study, unpaired analysis). Late Post PE means sample obtained at a later time point after PE and Late Post C means control sample obtained at same time point as the Late Post PE sample.

FIG. 8.

Overview of number of proteins significantly upregulated (upper circles) and downregulated (lower circles) in Early Post C and Late Post C compared with Pre C (validation study, unpaired analysis). Pre C means control sample obtained prior to a sham surgery, Post C means sample obtained early after sham surgery and Late Post C means samples obtained at a later timepoint after sham surgery.

FIG. 9.

Overlap of upregulated proteins in Early Post PE (left circle) and Late Post PE (right circle) compared with Pre PE and Early Post PE compared with Early Post C (upper circle) (validation study, unpaired analysis).

FIG. 10.

STRING analysis of positive markers for PE. Nodes are proteins and edges show the degree of interaction. The number of edges are significantly higher than expected for a random set of proteins. The proteins are at least partially biologically related with a large part of proteins in the extracellular region (dark grey nodes) (validation study, paired and unpaired analysis).

FIG. 11.

Overlap of downregulated proteins in Early Post PE (left circle) and Late Post PE (right circle) compared with Pre PE, in Early Post PE compared with Early Post C (upper circle) and in Late Post PE compared with Late Post C (lower circle) (validation study, unpaired analysis).

FIG. 12.

STRING analysis of negative markers for PE. Nodes are proteins and edges show the degree of interaction. The number of edges are significantly higher than expected for a random set of proteins. The proteins are at least partially biologically related with a large part of proteins in the extracellular region (dark grey nodes) (validation study, paired and unpaired analysis).

FIG. 13.

Clinical data, functional and invasive measures at baseline and 30 minutes after pulmonary embolism for PE animals and at index times for the negative controls (mean±SEM) (validation study).

FIG. 14.

Protein concentrations in the Exhaled Breath Condensate (EBC) and in the exhaled air of the validation study.

FIG. 15.

Positive markers for PE found in the validation study (un-paired analysis). PE: Pulmonary embolism.

FIG. 16.

Negative markers for PE found in the validation study (un-paired analysis). PE: Pulmonary embolism.

FIG. 17.

Fold changes and p-values from paired analysis of data from the validation study.

FIG. 18.

Fold changes and p-values for proteins in the validation study that was also observed in the pilot study. Values with *is based on paired analysis.

FIG. 19.

Amount of protein plotted against volume of exhaled air during the collection of Exhaled Breath Condensate (EBC) in the validation study.

FIG. 20.

Four proteins were upregulated in both Early Post PE and Late Post PE compared with Pre PE (represented in the intersection of the right and left circles); LYZ, AMY1A, ALB and BLMH, the latter was also upregulated in Early Post PE compared with Post C as depicted in the intersection of all three circles. Three proteins were upregulated in both Early Post PE compared with Pre PE and in Early Post PE compared with Post C (DSG1, SERPINB12, CSTA) (validation study, unpaired analysis). Pre PE means sample obtained prior to pulmonary embolism (PE), Post PE means sample obtained early after PE and Late Post PE means samples obtained at a later timepoint after PE. C refers to the control samples obtained at identical time points in control animals.

FIG. 21.

One of the four proteins that were upregulated in both Early Post and Late Post PE compared with Pre PE was downregulated in Late Post C compared with Pre C (LYZ) (validation study, unpaired analysis). Pre PE means sample obtained prior to pulmonary embolism (PE), Post PE means sample obtained early after PE and Late Post PE means samples obtained at a later timepoint after PE. C refers to the control samples obtained at identical time points in control animals.

FIG. 22.

One of the significantly upregulated proteins in Early Post PE compared with Post C was downregulated in Early Post C compared with Pre C (GGCT) (validation study, unpaired analysis). Pre C means control sample obtained prior to a sham surgery, Post C means sample obtained early after sham surgery and Late Post C means samples obtained at a later timepoint after sham surgery. Post PE means sample obtained early after PE.

FIG. 23.

Twelve proteins were downregulated in both Early Post PE and Late Post PE compared with Pre PE (9+3 represented in the intersection between the left and right circles); KATNAL2, LCN2, SERPINB4, ATMIN, IGHG1, TPI1, RPLP2, CALML3, CALML5, ALDOA, PPIB and C3—the latter 3 were also down regulated in Early Post PE compared with Early Post C (represented in the intersection between all three circles). These three plus thirteen proteins were downregulated in both Early Post PE compared with Pre PE and in Early Post PE compared with Post C; HSPA5, ARHGDIA, TKT, AKR1B10, PABPC1, ACTN4, ACTG1, HSP90AA1, TUBB4B, A2ML1, VCP, EEF1A1P5 and TUBA1B (validation study, unpaired analysis). Pre PE means sample obtained prior to pulmonary embolism (PE), Post PE means sample obtained early after PE and Late Post PE means samples obtained at a later timepoint after PE. C refers to the control samples obtained at identical time points in control animals.

FIG. 24.

Five proteins were downregulated in both Late Post PE compared with Pre PE and in Late Post PE compared with Late Post C (IGHG2, IGKC, IL36G, MDH1 and ERP29). Two proteins were downregulated in both Early Post PE and Late Post PE compared with Pre PE and Late Post PE compared with Late Post C (IGHG1, LCN2). One protein was downregulated in both Early Post Pre compared with Pre PE and Late Post PE compared with Late Post C (TYMP) (validation study, unpaired analysis). Pre PE means sample obtained prior to pulmonary embolism (PE), Post PE means sample obtained early after PE and Late Post PE means samples obtained at a later timepoint after PE. C refers to the control samples obtained at identical time points in control animals.

FIG. 25.

Two of the proteins significantly downregulated in both Early Post PE and Late post PE compared with Pre PE were significantly upregulated in Early Post C compared with Pre C (ALDOA and C3, represented in the dark grey/dark grey/left light grey intersection). Three of the downregulated proteins in Early Post PE compared with Pre PE were significantly up-regulated in both Early Post C and Late post C compared with Pre C (A2ML1, ACTG1 and HSP90AA1, represented in the light grey/light grey/left dark grey intersection). Two of the downregulated proteins in Early Post PE compared with Pre PE were significantly upregulated in Early Post C compared with Pre C (TUBA1B and EEF1A1P5 represented in the left dark grey/left light grey intersection). Five of the downregulated proteins in Early Post PE compared with Pre PE were upregulated in Late Post C compared with Pre C (marked in the right light grey/left dark grey intersection; TYMP, LGALS7, SFN, ENO1, GSTP1) (validation study, unpaired analysis). Pre PE means sample obtained prior to pulmonary embolism (PE), Post PE means sample obtained early after PE and Late Post PE means samples obtained at a later timepoint after PE. C refers to the control samples obtained at identical time points in control animals.

FIG. 26.

Six of the significantly downregulated proteins in Early Post PE compared with Post C was significantly upregulated in Early Post C compared with Pre C (TUBA1B, ALDOA, EEF1A1P5, PPIA, C3, YWHAZ). Four of the downregulated proteins in Early Post PE compared with Early Post C were upregulated in both Early Post C and Late Post C compared with Pre C (A2ML1, HIST2H2AC, ACTG1 and HSP90AA1). One of the significantly downregulated proteins in Late Post PE compared with Late Post C was significantly upregulated in Late Post C compared with Pre C (TYMP) (validation study, unpaired analysis).

FIG. 27.

Proteins in the condensate from the mechanical ventilator compared with the Pre PE and Pre C samples (N=19) (validation study). PE: Pulmonary Embolism. C: Control.

FIG. 28.

Proteins present at different amounts Early Post PE compared with Pre PE (N=14) (validation study, unpaired analysis). PE: Pulmonary Embolism

FIG. 29.

Proteins present at different amounts Late Post PE compared with Pre PE (N=14) (validation study, unpaired analysis). PE: Pulmonary Embolism.

FIG. 30.

Proteins present at different amounts Early Post PE compared with Post C (N=18) (validation study, unpaired analysis). PE: Pulmonary Embolism. C: Control.

FIG. 31.

Proteins present at different amounts Late Post PE compared with Late Post C (N=18) (validation study, unpaired analysis). PE: Pulmonary Embolism. C: Control.

FIG. 32.

Proteins present at different amounts Early Post C compared with Pre C (validation study, unpaired analysis). C: Control.

FIG. 33.

Proteins present at different amounts Late Post C compared with Pre C (validation study, unpaired analysis). C: Control.

FIG. 34.

Proteins present at different amounts Late Post PE compared with Pre PE in the animals treated with placebo (N=6) (validation study, unpaired analysis). PE: Pulmonary Embolism.

FIG. 35.

Proteins present at different amounts Late Post PE treated with a vasodilatory drug (VD) compared with Late Post PE treated with placebo (N=13, because both first and second replicate of the Late Post PE sample from pig number 6 was missed, see methods section) (validation study, unpaired analysis). PE: Pulmonary Embolism.

FIG. 36.

Enrichment component analysis of positive markers of PE (validation study). PE: Pulmonary Embolism.

FIG. 37.

Enrichment component analysis of negative markers of PE (validation study). PE: Pulmonary Embolism.

FIG. 38.

Ten proteins were upregulated in Early Post PE compared with Pre PE in both the paired and unpaired analysis (TF, ALB, AMY1A, LYZ, LCN1, BLMH, CSTA, SERPINB12, DSG1, LTF). Fourtytwo proteins were downregulated in Early Post PE compared with pre PE in both paired and unpaired analysis (PABPC1, VCP, TYMP, KATNAL2, TUBA1B, A2ML1, EPPK1, LCN2, SERPINB4, ALDOA, TUBB4B, MYH9, EEF1A1P5, ACTN4, EEF2, IGHG1, LGALS7, SFN, GM2A, ENO1, PKP1, GSTP1, TPI1, ACTG1, SERPINB3, DSP, HSP90AA1, CALML3, CFL1, CALML5, HSPB1, ARHGDIA, HSPA5, CRABP2, IGHG2, ITIH4, ATP5B, RPS7, POF1B, RPL22, EZR, GD12). Pre PE means sample obtained prior to pulmonary embolism (PE) and Early Post PE means sample obtained early after PE.

FIG. 39.

Ten proteins were upregulated in Late Post PE compared with Pre PE in both the paired and unpaired analysis (TF, ALB, FN1, DSG1, APOA1, TOM1, NAGK, ACTB, SLC25A6, DSC3). Fourteen proteins were downregulated in Late Post PE compared with pre PE in both paired and unpaired analysis (VCP, TUBA1B, LCN2, SERPINB4, PPIB, IGHG1, PKP1, TPI1, SERPINB3, RPLP2, CALML3, CALML5, IGLC3, IGKC). Pre PE means sample obtained prior to pulmonary embolism (PE) and Late Post PE means samples obtained at a later timepoint after PE.

FIG. 40.

A STRING analysis (including bioinformatic characterization), in which intracellular proteins (proteins localized to the cytoplasm, intracellular membrane-bounded organelles, intracellular non-membrane-bounded organelles or to the cytosol) differentially expressed in EBC were identified. Nodes are proteins and edges show the degree of interaction. The human genes HBA1 and HBA2 are used instead of the pig gene HBA.

FIG. 41

Intracellular proteins. Proteins identified in the present invention that are localized to the cytoplasm, intracellular membrane-bounded organelles, intracellular non-membrane-bounded organelles or cytosol. PE: Pulmonary Embolism.

FIG. 42

Proteins associated with hemoptysis and major plasma proteins, which were found to be upregulated during the PE in the experimental setup. PE: Pulmonary Embolism.

FIG. 43

A STRING analysis where proteins associated with neutrophil degranulation, platelet degranulation and acute inflammatory response were identified. The bioinformatics were based on differentially expressed proteins (p<0.1) and solely proteins with positive fold changes were included. Nodes are proteins and edges show the degree of interaction.

FIG. 44

A STRING analysis where proteins associated with neutrophil degranulation, platelet degranulation and the innate immune system were identified. The bioinformatics were based on differentially expressed proteins (p<0.1) and solely proteins with positive fold changes were included. Nodes are proteins and edges show the degree of interaction.

FIG. 45

Upregulated proteins associated with postembolic pulmonary inflammation.

FIG. 46

Positive biomarkers based on (increasing) p-values. Proteins that were up-regulated with an associated p-value of either <0.01, <0.05, or 0.1 in the following comparisons Early Post/Pre (paired and un-paired) and Late Post/Pre (paired and un-paired) and Early Post/Early Post_c and Late Post/Late Post_c. Statistics were based on student's t-test without further correction for multiple comparison.

FIG. 47

Favourite down-regulated biomarkers based on dataquality, fold-changes, p-values and literature findings.

FIG. 48

The figure shows prediction models with the cross-validation (y-axis) for each value of lambda (x-axis). The models also contain an alpha value that was regulated by the researcher during model optimization. (A) Misclassification vs. Lambda values for Pre PE vs Early post PE predition. (B) Misclassification vs. Lambda values for Pre PE vs late post PE predition. PE: Pulmonary Embolism.

The prediction model included PE animals, but not the negative controls (4 animals). Proteins present in more than 70% of the samples were included and any missing values were imputed using k-nearest neighbour. Lambda is the weight of the penalty; higher values forces the model to choose fewer parameters. Leave-one-out cross-validation was done for a variety of lambdas. Thus, the observations were suspended one-by-one and trained the model based on the remaining observations to see how the model performs. PE: Pulmonary Embolism.

FIG. 49

Proteins from the prediction models of pulmonary embolism. Coefficient: a positive value means that an increase in expression increases the probability of PE, whereas a negative value means that an increase in expression decreases the probability of PE. PE: Pulmonary Embolism.

FIG. 50

Proteins that were up-regulated with an associated p-value of either <0.01, <0.05, or 0.1 in more than one comparison in the dataset, where the statistics were based on a students t-test, paired and unpaired, without further correction for multiple comparisons. PE: Pulmonary Embolism.

### EXAMPLES

### Example 1: Validation Study

The study animals were female Danish Landrace pigs weighing 60 kg. Sham pigs were included as negative controls that underwent the exact same protocol regarding medication, intubation, monitoring, ventilation, placement of sheaths etc. as the pigs who got a pulmonary embolism, except for 8 pigs that also received vasodilators. In the step where the pulmonary embolism was infused, the negative controls were solely given the bolus of isotonic saline.

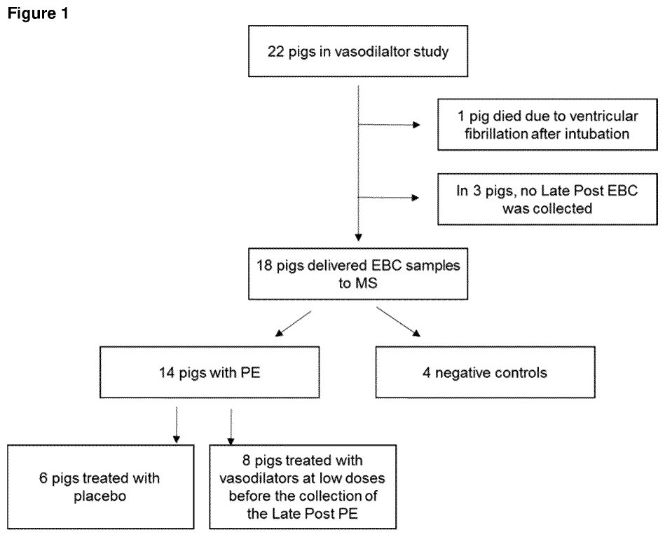

In total 22 pigs were included in the study; 4 negative controls and 18 in whom an autologous pulmonary embolism was induced (FIG. 1). The pigs were born and bred at a local specific pathogen free farmer and kept in quarantine for seven days in the research animal facility at Aarhus University before the experiment. The research animals arrived at the research facility at eight o'clock in the morning of the experimental day.

Briefly, anesthesia was induced by intravenous Etomidate (0.5 mg/kg, Hypnomidate®Janssen Parmaceutical, Belgium) and maintained after intubation (ID 7.5 mm, Unomedical, Malaysia) by continuous intravenous infusion of Propofol (2 mg/kg/hour, Propolipid, Fresenius Kabi, Germany) and Fentanyl (5 μg/kg/hour, Hamlen Pharma, Germany). Upon intubation pressure controlled volume gated mechanical ventilation (GE Datex-Ohmeda S/5 Avance) with non-humidified air was initiated. To correct hypoxemia in association to intubation, the fraction of inspired 02 was initially set to 1.0, but reduced to 0.3 soon after. The tidal volume was set to 8 ml/kg and the respiratory rate to 16 breaths per minute, no positive end-expiratory pressure was applied. The end tidal CO₂ before induction of the pulmonary embolism was maintained at 5.0 kPa by increasing the respiratory rate if necessary. The core temperature was maintained at 38-39° C. by use of the Bair Hugger™ normothermia system, monitored continuously by a rectal thermometer. Three intravascular sheaths were applied guided by ultrasound before the first collection of exhaled breath condensate: A 7-French sheath in the femoral vein for continuous isotonic saline infusion and drawing of venous blood samples, a 6-French sheath was inserted in the contralateral femoral artery for continuous monitoring of blood pressure and heart rate and arterial blood sampling. A 7-French sheath was placed in the left external jugular vein for infusion of anesthetics. After the first collection of exhaled breath condensate, 60 mL of blood was drawn from the left external jugular vein and distributed to two ¾″ uncoated extra corporal tubes for spontaneous coagulation forming two autologous emboli at room temperature over three hours. A 26-French Dry-Seal sheath (Gore Medical, USA) was placed guided by fluoroscopy via the right external jugular vein to the superior vena cava ending the level of the right atrium for administration of the emboli and right sided heart catheterization before and after induction of pulmonary embolism. Serum was discarded before each embolus was transferred to the 26-French sheath through which it was flushed by isotonic saline to be pushed into the pulmonary arteries. Invasive hemodynamic and functional measures plus blood samples were obtained immediately before the administration of the pulmonary embolism, 30 minutes after the pulmonary embolism and 2% hours after. The pigs were euthanized by intravenous injection of Phenobarbital (67 mg/kg, Exagon®, vet, Richter Pharma, Austria) at the cessation of the experiment.

Collection and Storage of the Exhaled Breath Condensate

The exhaled air was cooled and the resulting condensate collected in a polypropylene collection tube precooled to −80° C. (RTubeVent, Respiratory Research Inc., Austin, Tex.). The collection tube and the associated cooling sleeve were inserted between the expiratory limb of the Y-connector and the expiratory limb of the mechanical ventilator. In total six EBC samples were collected from each pig; two before induction of pulmonary embolism (Pre PE) (after all the sheaths were in place), two new samples were collected starting 30 minutes after the induction of pulmonary embolism (Early Post PE) and finally two samples were collected starting 2% hours after pulmonary embolism (Late Post PE) and at corresponding times in the negative controls (Pre C, Early Post C and Late Post C). In eight of the pigs, in whom pulmonary embolism was induced, two low doses of vasodilatory drugs (NO, Sildenafil, or Riociguate) were given before the collection of the Late Post PE sample because this study was an amendment protocol to a pharmacological study (see FIG. 1).

In the pilot study, we observed that an initial collection temperature of −80° C. prompted collection periods of 18 minutes, and accordingly the collection time for each sample in this study was planned to be 18 minutes as a minimum. The heat and moisture exchange filter was disconnected during collection of the EBC. A condensate sample from the mechanical ventilator was collected by inserting a sterile set of ventilator hoses connected to an adult breathing circuit with the Evaqua™ technology (RT380/RT385, Fisher & Paykel Healthcare, New Zealand). The moisture chamber was filled with sterile, isotonic sodium chloride. The resulting condensate was collected by insertion of the collection tube covered by the aluminum sleeve precooled to −80° C. inline at the expiratory limb directly at the mechanical ventilator. Gowns, surgical hairnets and new nitrile gloves were worn whenever the EBC were handled. Immediately after collection, the EBC was transferred by sterile pipettes to sterile 2.0 ml low temperature freezer vials and immediately stored at −80° C.

Preparation of EBC Samples for Mass Spectrometry

The volume of EBC was estimated by weighing assuming that 1 gram equals 1 mL. Samples were vacuum centrifuged and re-dissolved in 120 μL digestion buffer (0.5% SDC, 20 mM TEAB). The fluorescence (Excitation at 295 nm, Emission at 350 nm) was measured in microtiter trays using 100 μL. A standard curve was constructed from tryptophan. The protein concentration was estimated under the presumption that 1 g of protein corresponds to 0.0117 g of tryptophan as it is the case for human and mouse protein samples.

Samples were incubated with tris(2-carboxyethyl)phosphine (TCEP) at 55° C. for 1 h. Iodoacetamide was added and samples incubated for further 30 min. at room temperature protected from light before being added to Microcon 30K centrifugal filter devices (Merck Milipore Ltd., Tullagreen, IRL) and centrifuged at 14,000 g for 15 min. Digestion buffer was added to the filter units two times with centrifugation. Finally 50 μL of digestion buffer was added together with trypsin and mixed at 600 rpm in a thermomixer for 1 min. The units were incubated in a wet chamber at 37° C. overnight. Samples were then centrifuged for 14,000 g for 10 min. into new collection tubes. The filters were rinsed with 100 μL digestion buffer twice. Trifluoroacetic acid (TFA) were added to a final concentration of 0.5% (v/v). An equal volume of ethyl acetate was added, shaken for 1 min. and centrifuged at maximum speed for 2 min. The lower phase was saved by removal of the upper phase. The extraction was repeated two times. The samples were dried in a vacuum centrifuge and re-suspended in 120 μL 100 mM TEAB. The peptide concentration was measured by fluorescence. Samples were vacuum dried, resuspended in 0.1% (v/v) formic acid at a concentration of 0.05 μg/μL. Three μL (0.15 μg) was loaded for each run and samples were analyzed in replicates. Some samples gave problems during the LC run, mostly because of pressure increases. Due to these problems, not all files were run twice. The first replicate of the Late Post PE in pig number 6 and the Pre PE in pig number 8 plus the second replicate of the Late Post PE in pig number 6, Pre PE in pig number 7 and Early Post PE in pig number 19 were thereby missed.

Nano Liquid Chromatography-Tandem Mass Spectrometry (nLC-MS/MS)

The peptide mixture was separated by nLC on an UltiMate 3000 (Thermo Scientific) coupled to an Orbitrap Fusion mass spectrometer (Thermo Scientific) through an EASY-Spray source (Thermo Scientific). A trap column (300 μm×5 mm, C18 PepMap100, 5 μm, 100 Å, Thermo Scientific) was used to load the sample. An analytical column (EASY-Spray Column, 750 mm×75 μm, PepMap RSCL, C18, 2 mm, 100 Å, Thermo Scientific) was used to separate peptides. A 90 min. gradient was formed by mixing buffer A (0.1% formic acid) with buffer B (80% acetonitrile, 20% water, 0.1% formic acid). The following amount of buffer B was used, 6% (0 min.), 16% (3 min.), 30% (55 min.), 60% (67 min.), 99% (70 min.), 99% (80 min.), 6% (81 min.), 6% (90 min.). The universal method setting was used for mass spectrometry detection with full Orbitrap scans (m/z 400-1500) at a resolution of 120,000. Automatic gain control (AGC) target of 4×10⁵ and a maximum injection time of 50 ms was used. The cycle time was 3 sec. The most intense precursors were selected with an intensity threshold of 5×10³ in top speed data dependent mode. MS² scans were performed in the linear ion trap in auto scan range mode with CID energy set at 35%, an AGC target of 2×10³ and a maximum injection time of 300 ms. The precursor ions with charge states 2-7 were isolated using the quadrupole with an isolation window of 1.6 m/z. Dynamic exclusion was set to 60 s.

Identification and Label-Free Quantification of the Proteins

The raw MS datafiles were searched against Uniprot databases, Sus scrofa and Homo sapiens downloaded on Feb. 22, 2017 using MaxQuant (v1.5.5.1) for label free quantification (LFQ). Fixed modification was carbamidomethyl (C). The false discovery rate was set to 1% for PSM, protein and site and the LFQ minimum ratio count was set at 1. MS/MS was required for LFQ comparisons. Unique and razor peptides, unmodified and modified with oxidation (M) or acetyl (protein N-terminal), were used for protein quantification with a minimum ratio count of 2. The match between runs function was activated. Reverse sequences were used for decoy search. Contaminant sequences were included. The results were entered into Perseus (v1.5.8.5) for further filtering and statistical analysis. Proteins identified as potential contaminants, only identified by site or by the reverse part of the database were removed. After filtering in Perseus, 45 proteins were only identified by site, 9 were identified in the reverse database and 76 were potential contaminants leaving 897 proteins for further analysis. The proteins included in the subsequent analyses are not necessarily represented in all the EBC samples since we chose not to filter by valid values (i.e. we did not remove proteins if they were not found in all the EBC samples). All peptides were used for protein quantification. The LFQ values were log 2 transformed, and the arithmetic mean of the technical duplicates were calculated. The EBC samples were then categorized as either Pre PE, Early Post PE or Late Post PE in the PE animals, or Pre C, Early Post C or Late Post C in the negative controls. Changes of the proteins of the Pre PE and Pre C samples compared with the condensate from the mechanical ventilator were calculated as fold changes (i.e. amount of a certain protein in Pre PE or Pre C samples divided by the amount of the same protein in mechanical ventilator condensate) and a one-sample t-test was used to calculate associated p-values. Unpaired t-tests were used to calculate the differences and associated p-values for the proteins in the Early Post PE versus the Early Post C and for the Late Post PE versus the Late Post C samples. We calculated the differences and associated p-values using unpaired t-tests in the Early Post PE versus Pre PE and Late Post PE versus Pre PE in the PE animals and Early Post C versus Pre C and Late Post C versus Pre C in the negative controls. Additionally, paired t-tests were used to calculate the differences and associated p-values for the proteins in the Early Post PE versus Pre PE and Late Post PE versus Pre PE samples. To assess possible effects of the vasodilatory drugs on the results in analysis of Late Post PE samples, we included a subgroup analysis of pigs treated with placebo (FIG. 34) and compared the Late Post PE from pigs treated with vasodilators with Late Post PE samples from pigs treated with placebo (FIG. 35). Linear fold changes were calculated by taking the base 2 to the power of the log 2 transformed fold changes. Fold change >1 indicates that the protein had higher LFQ values in the particular type of sample (pigs compared with the mechanical ventilator; in the PE animals as compared with the negative controls; in the Early Post PE or Late Post PE as compared with the Pre PE in the PE pigs; and Early Post C or Late Post C compared with the Pre C in the negative controls, respectively). Fold change <1 indicate that the proteins had lower LFQ values in the particular type of sample in the before mentioned comparisons. A linear fold change of 17.1 is referred to as a 17-fold higher amount/upregulation of the protein, while a fold change of 0.16 corresponds to a protein amount of 1/6. Proteins with p-values <0.10 were considered significantly changed after PE.

Statistics Concerning Clinical Parameters and Protein Concentrations

In analyses where we compared the means of the PE animals and the negative controls, data were analyzed as independent samples by the Students t-test. The assumption of normality was checked by inspection of Q-Q plots, and when violated the Wilcoxon rank-sum test was used. The functional and invasive measures obtained at baseline (Pre PE) and 30 minutes after induction of PE (Early Post PE) were analyzed as paired samples based on the Student's t-test for PE animals and negative controls. The assumptions of equal distributions of the differences and normality of the distribution were checked by visual inspection of relevant plots. Estimates are presented as means and standard error of the mean (SEM). The volume of exhaled air during the EBC collection was calculated as collection time*tidal volume*respiratory frequency. The latter two parameters were readily available from the monitor of the mechanical ventilator and were noted at Pre PE/Pre C, after which the settings were unaltered. A one way-analysis of variance (ANOVA) was used to determine whether the EBC collection time differed Pre PE, Early Post PE and Late Post PE. The underlying assumptions of normality and variance homogeneity were checked by QQ plots and Bartlett's test, respectively.

Bioinformatic Analysis

STRING (string-db.org) was used for bioinformatic analysis of the differentially expressed proteins. Positive markers and negative markers with the gene names as given in tables 1 and 2 were entered for recognition as human genes. Few of these were not recognized by STRING and the analyses were based on the recognized genes.

Results

The Research Animals

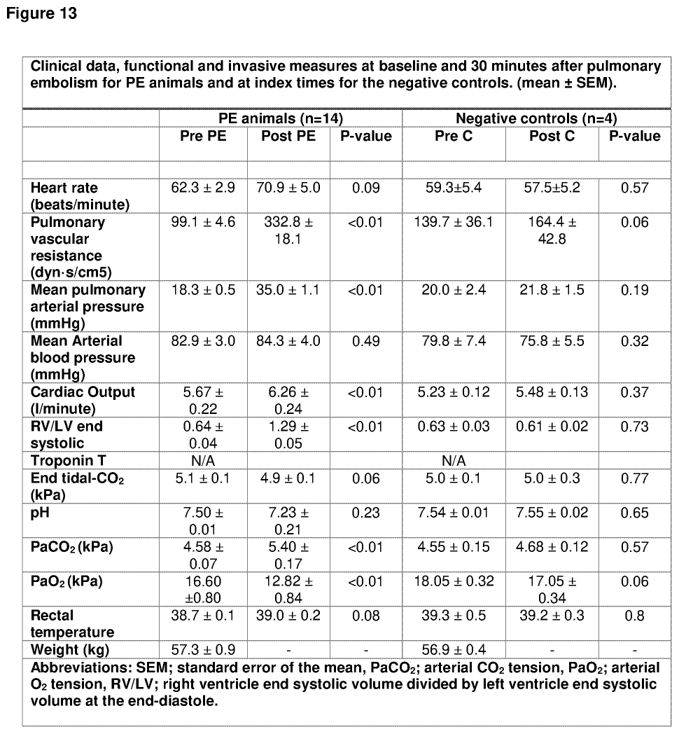

Twenty-two pigs were included in the study. For three of the pigs the Late Post samples were not collected and one of the pigs died due to ventricular fibrillation after intubation. Thus, Pre PE, Early Post PE and Late Post PE samples were obtained from 14 PE animals and Pre C, Early Post C and Late Post C EBC samples from four negative controls (FIG. 1). Mean temperature in the research facility was 22.4° C. (min 21.3, max 23.5). FIG. 13 depicts basic data for the pigs showing no significant differences between PE animals and negative controls.

Troponin T, the pulmonary vascular resistance, the mean pulmonary artery pressure and the end systolic volume in the right ventricle was significantly higher Early Post PE compared with Pre PE. In the negative controls, these parameters were largely unaltered during the procedure. The cardiac output did not decrease and the mean arterial blood pressure was unaltered Early Post PE (FIG. 13), but the PE animals showed significant signs of ventilation/perfusion mismatch Early Post PE with increased arterial CO₂ partial pressure and decreased arterial O₂ partial pressure and accordingly lower arterial pH Post PE compared with Pre PE, while in the negative controls no significant signs of ventilation/perfusion mismatches were observed (FIG. 13). Taken together, the PE animals had hemodynamic and biochemical signs of right ventricular strain but no signs of acute heart failure, corresponding to intermediate-high risk PE, while in the negative controls the hemodynamics and troponin T was unaltered.

Timing of EBC Collection and Induction of PE

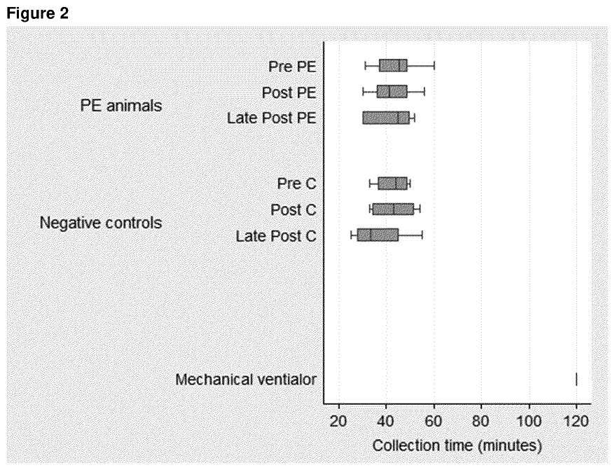

The mean collection time for the two collection tubes for the Pre PE/Pre C samples was 48 minutes±2, no difference between PE animals and negative controls (p-value 0.77), for the Early Post PE/Early Post C EBC samples 42 minutes±2, no difference between PE animals and negative controls (p-value 0.81) and for the Late Post PE/Late Post C EBC samples 41 minutes±2, no difference between PE animals and controls (P-value 0.35). The mean collection times for the EBCs Pre PE, Early Post PE and Late Post PE, respectively Pre C, Early Post C and Late Post C are depicted in FIG. 2, the one way-ANOVA showed no significant differences between the collection periods in the PE animals (p-value 0.76) or in the negative controls (p-value 0.64). Taken together, all time points and time spans in the experiment were similar for PE animals and negative controls. The mechanical ventilator yielded 5.86 mL condensate per 1000 liter “expired” air and therefore, a total collection period of 120 minutes was necessary in order to obtain a volume of condensate (3.9 mL) similar to the pigs, i.e. considerably longer than the collection times of EBC from the pigs (FIG. 2).

The Protein Concentration in the Exhaled Breath

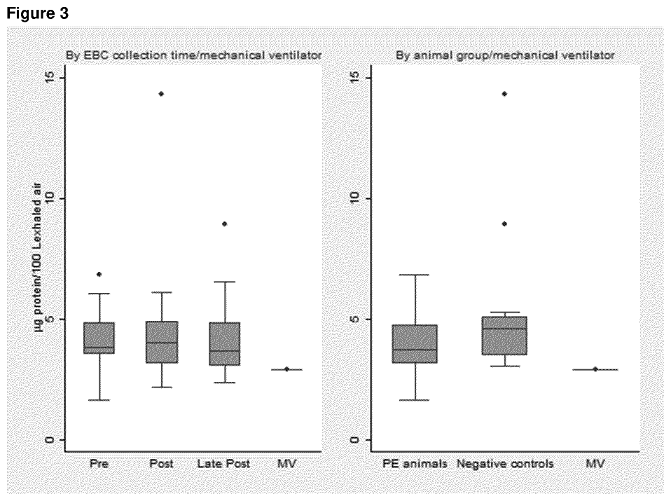

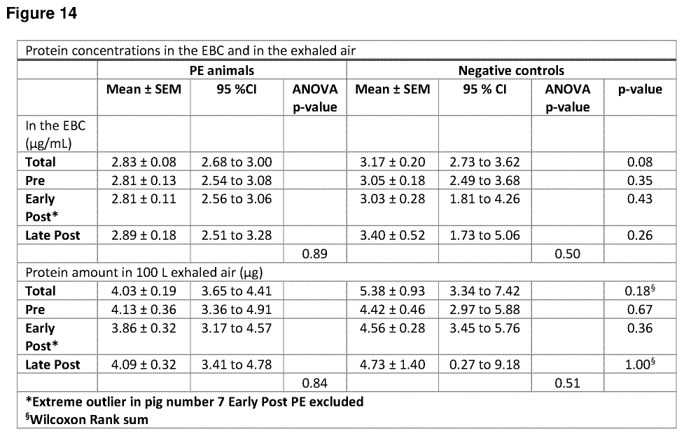

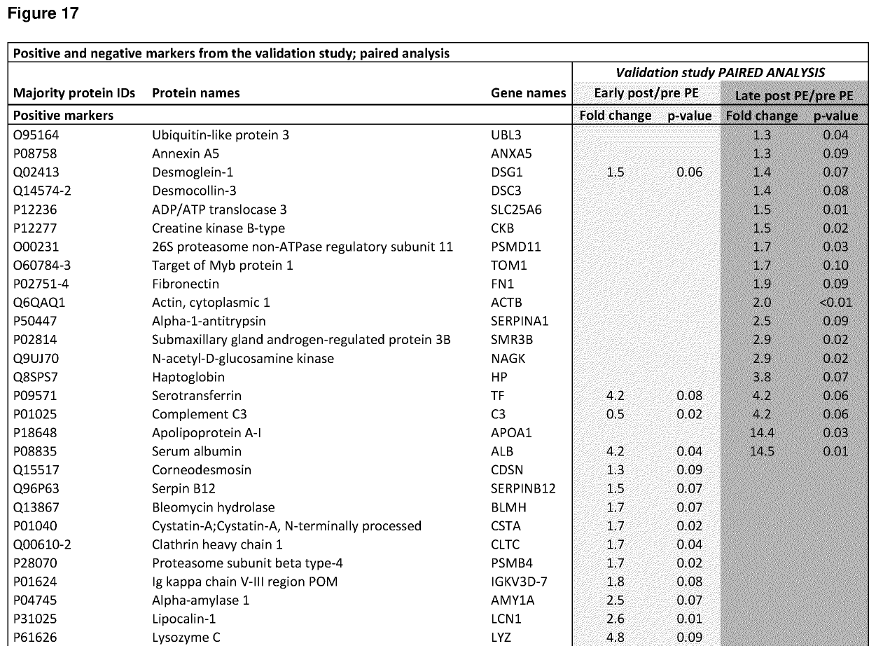

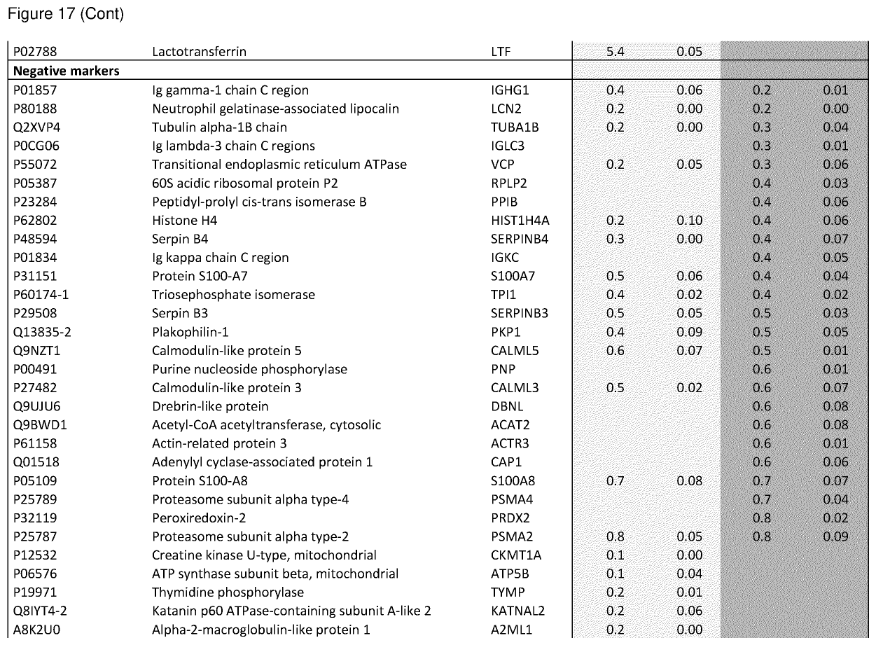

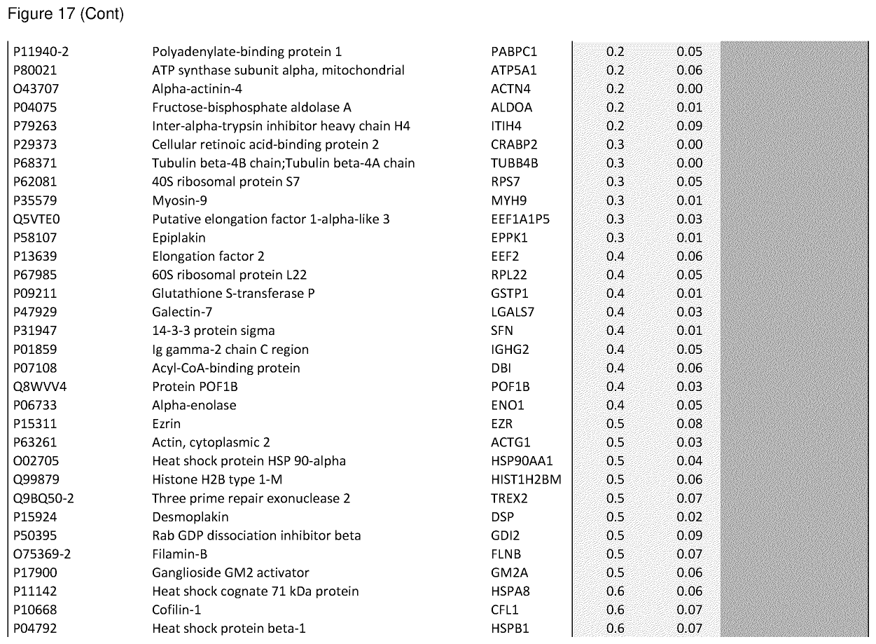

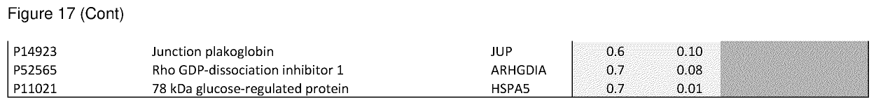

The mean concentration of proteins in the exhaled breath was 4.28 μg/100 l exhaled breath±0.26, which did not differ in PE animals and negative controls (Wilcoxon rank-sum p-value=0.12) or according to the collection times (FIGS. 3 and 14). In the mechanical ventilator 2.89 μg protein was collected per 100 liter of “exhaled air” which was lower compared with the pigs (FIG. 3). The increasing temperature of the aluminum cooling sleeve and subsequent efficiency of the condensation affected the duration of the collection period. The total protein amount was hence not markedly dependent of the total volume of exhaled air in our study (FIG. 17).

The Ventilators Condensate and the EBC

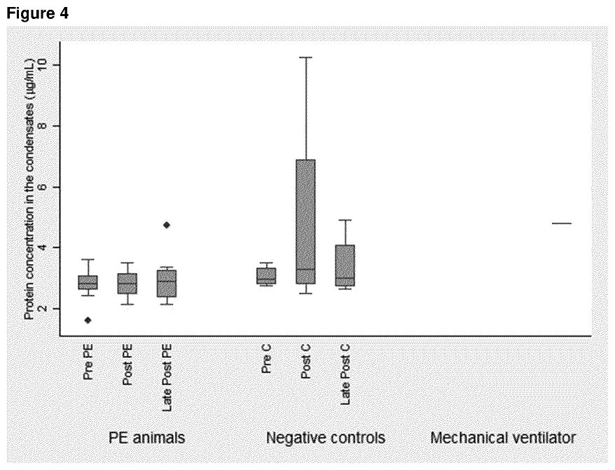

The protein concentration in the condensate from the mechanical ventilator was 4.81 μg/mL, which was higher compared with the protein concentrations in the EBCs (FIG. 4). Mean protein concentration in the EBC was 2.83±0.08 μg/mL for the PE animals, no difference between pre PE, Early Post PE and Late post PE (ANOVA p-value 0.89). For the negative controls, the mean protein concentration in the EBCs was 3.17±0.20 μg/mL and no difference between the different collection time groups (ANOVA p-value 0.50) (FIG. 14). The protein concentration in the EBC did not differ in PE animals compared with negative controls (FIGS. 4 and 14).

The Proteins in the Condensate from the Mechanical Ventilator and the EBCs

From all EBC samples and the condensate from the mechanical ventilator a total of 897 different proteins were detected after filtering in Perseus. The mean number of identified proteins in the EBCs was 249 (range 53-687).

Of the 897 proteins, 286 were also identified in the condensate from the mechanical ventilator; no proteins were identified in the mechanical ventilator solely (FIG. 25). 41% of the proteins identified in both pigs and mechanical ventilator (118 of the 286) were present at higher amounts in the EBC from the pigs, 19% of the proteins (55 of the 286) were present at lower amounts in the EBC from the pigs. For the remainder 40% of the proteins (113 of the 286) the amount was not significantly different in the EBC from the pigs compared with the mechanical ventilator (FIG. 25).

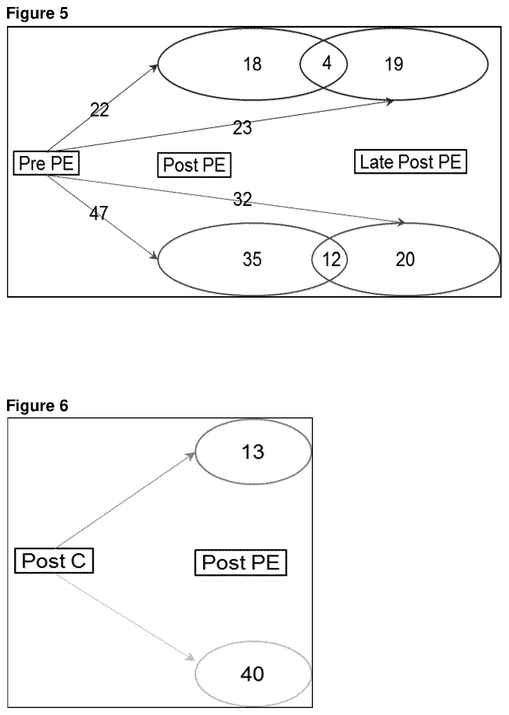

In the EBC collected in pigs Early Post PE and Late Post PE, more than a hundred proteins were present at significantly altered amounts compared with the Pre PE (FIGS. 5, 26 and 29). In Early Post PE compared with Pre PE, 22 proteins were significantly upregulated while 47 were significantly downregulated based on the unpaired t-test (FIG. 32). In Early Post PE compared with Pre PE, 14 proteins were significantly upregulated compared with Pre PE while 54 were significantly downregulated based on the paired t-test. Ten of the 14 up-regulated proteins from the paired analysis comparing Early Post PE with Pre PE were also identified in the un-paired analysis (FIG. 38). Fourty-two of the 54 proteins identified downregulated proteins in the paired analysis comparing Early Post PE with Pre PE were also identified in the un-paired analysis (FIG. 38). In the Late Post PE compared with Pre PE, 23 proteins were significantly upregulated while 32 were significantly downregulated based on the unpaired t-test (FIG. 33). In Late Post PE compared with Pre PE,17 proteins were significantly upregulated compared with Pre PE while 25 were significantly downregulated based on the paired t-test. Ten of the 17 up-regulated proteins from the paired analysis comparing Late Post PE with Pre PE were also identified in the un-paired analysis (FIG. 39). Fourteen of the 25 proteins identified downregulated proteins in the paired analysis comparing Late Post PE with Pre PE were also identified in the un-paired analysis (FIG. 39). Changes ranged from 17 fold higher amounts to reduction to 1/6 in the Early Post PE and Late Post PE samples compared with the Pre PE samples (FIGS. 26 and 27).

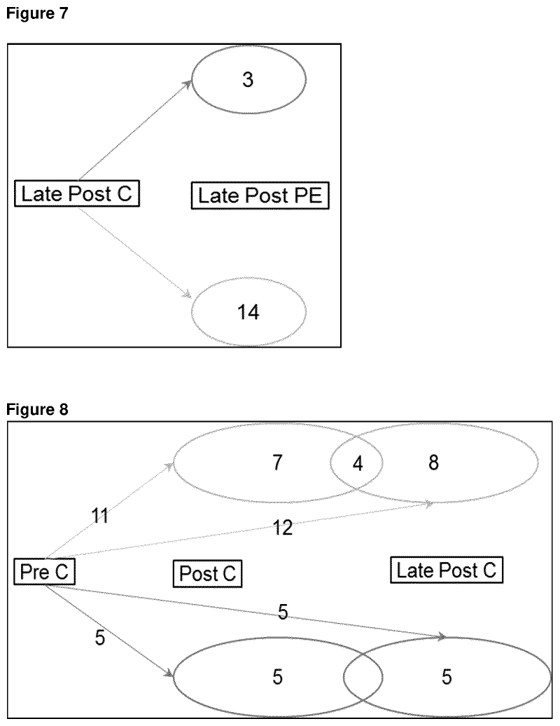

In Early Post PE compared with Early Post C, 13 proteins were upregulated (i.e. were present at significantly higher amounts in PE compared with controls) and 40 were downregulated (i.e. were present at significantly lower amounts in PE compared with controls, FIGS. 6 and 28). In Late Post PE compared with Late Post C, 3 proteins were significantly upregulated in PE compared with controls while 14 were significantly downregulated (FIGS. 7 and 29).

In analysis of samples from the controls (FIGS. 8, 30 and 31), 11 proteins were significantly upregulated in Post C compared with Pre C, while 5 were downregulated. In Late Post PE, 12 proteins were upregulated compared with Pre C, while 5 were downregulated.

Criteria for Identification of Biomarkers

In order to minimize the risk of overlooking biomarkers we prioritized that the risk of making a type 2 error should be minimal ignoring a concomitant higher risk of type 1 error and we therefore defined the following criteria for identification of positive and negative biomarker candidates:

Positive Markers

The criteria for selection as “positive marker” for PE (i.e. a protein that would be present in higher amounts in case of PE) were:
I. Proteins significantly upregulated in the Early Post PE or Late Post PE samples compared with the Pre PE samples (represented in the upper circles of FIG. 5) and not upregulated in Early Post C or late Post C compared with Pre C (not represented in the upper circles of FIG. 8).
II. Proteins significantly upregulated in Early Post PE compared with Post C (represented in the upper circle of FIG. 6) and not upregulated in Early Post C compared with Pre C (not represented in the upper circles of FIG. 8).
III. Proteins significantly upregulated in Late Post PE compared with Late Post C (represented in the upper circle of FIG. 7) and not upregulated in Late Post C compared with Pre C (not represented in the upper part of FIG. 8).
IV. Proteins significantly upregulated in the Early Post PE or Late Post PE compared with Pre PE in the paired analysis.

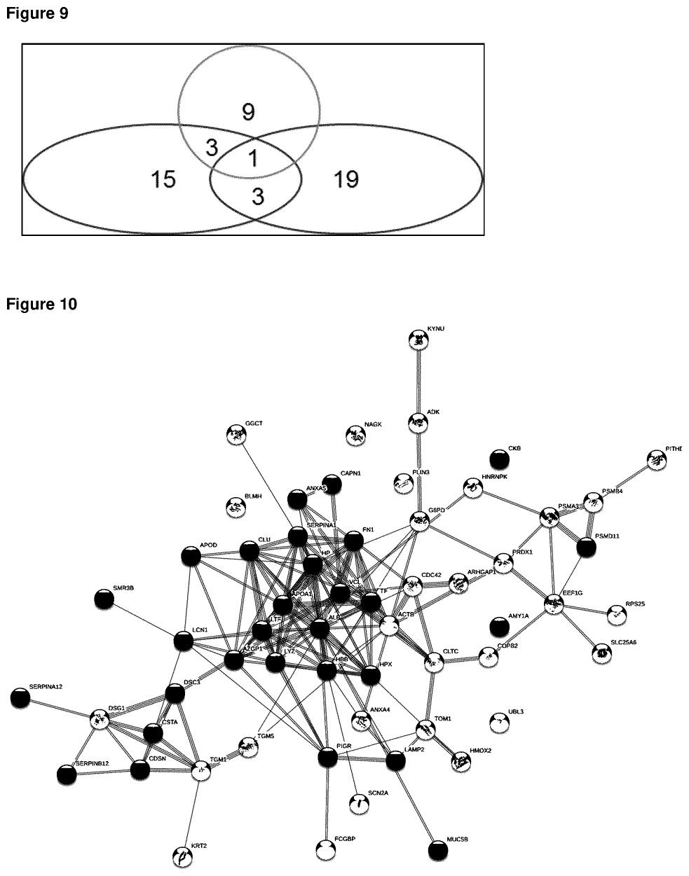

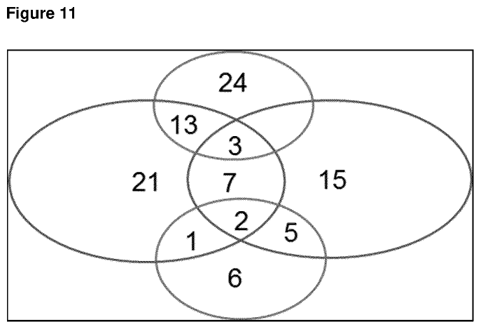

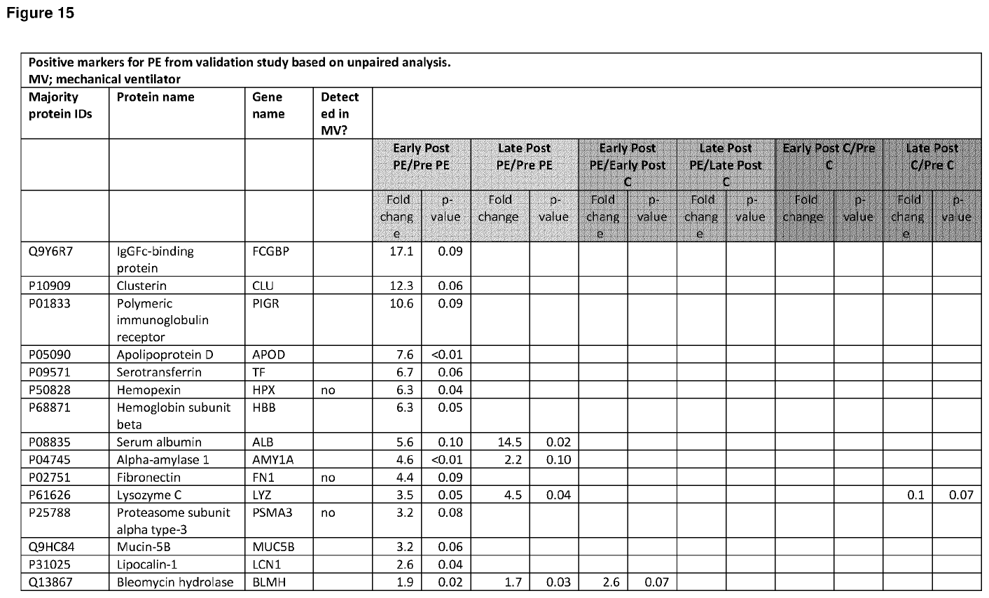

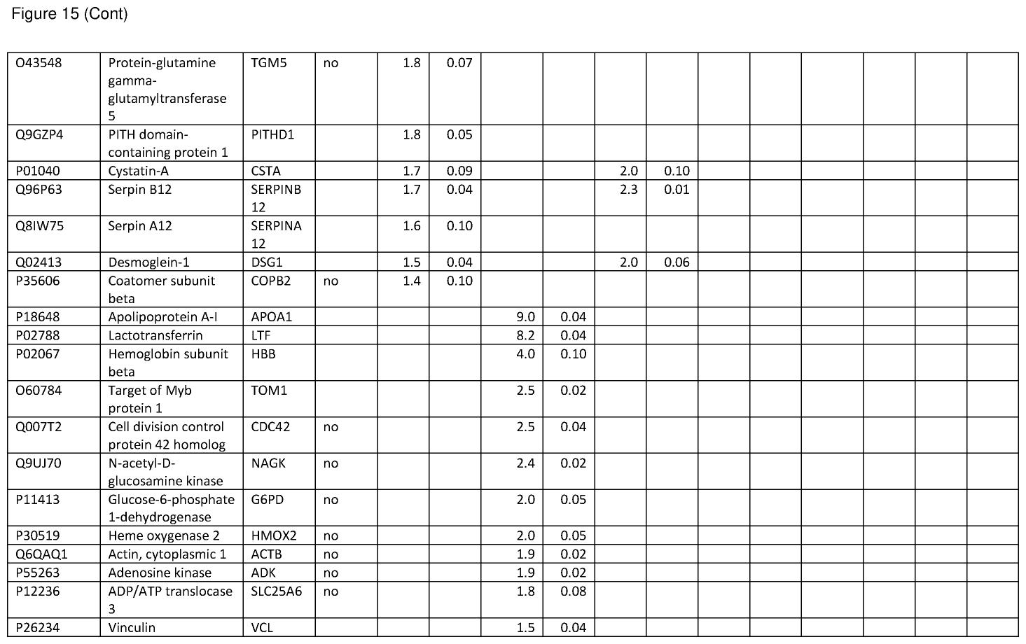

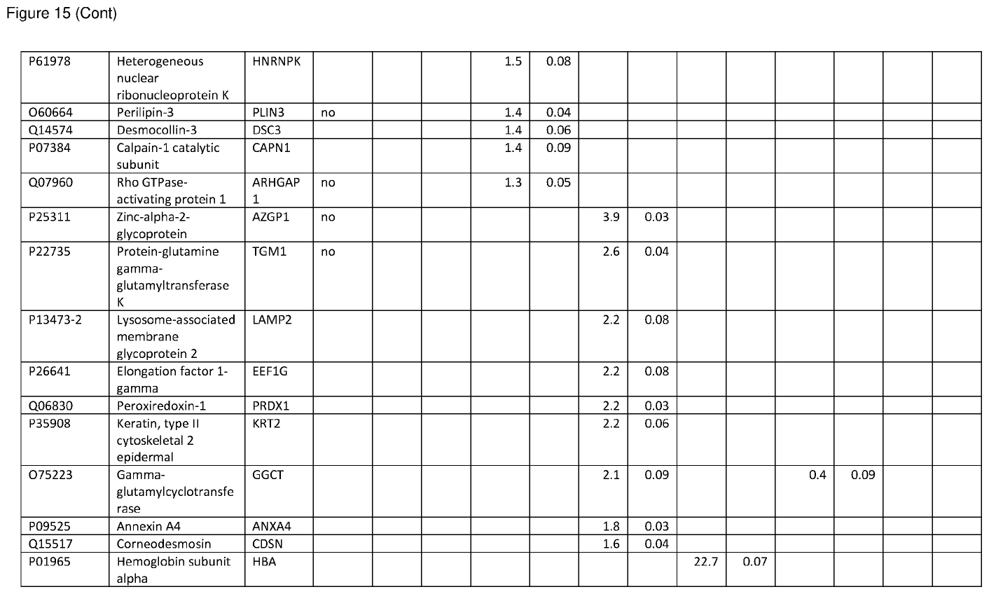

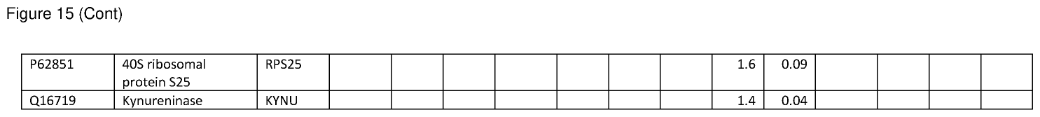

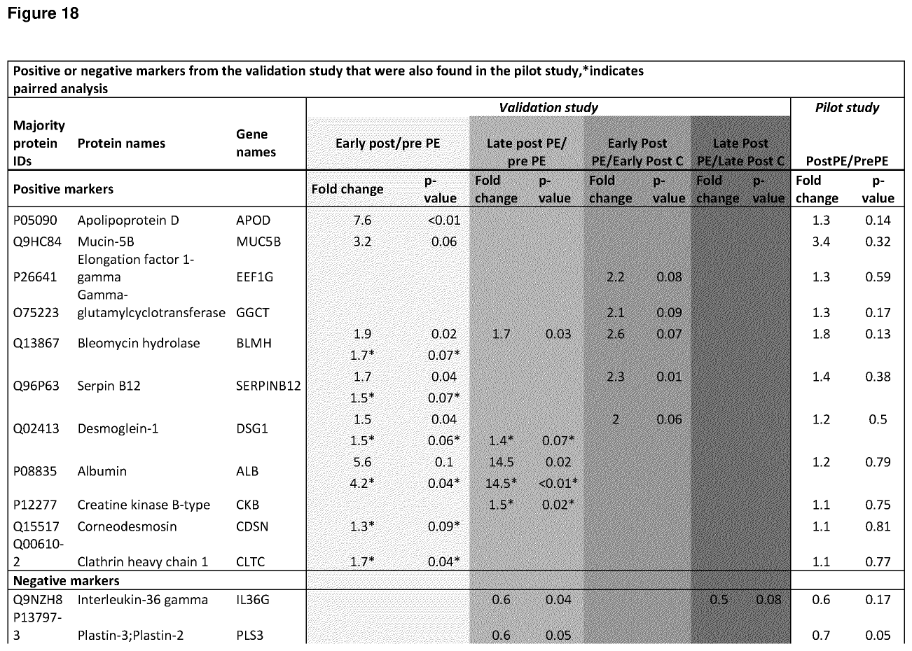

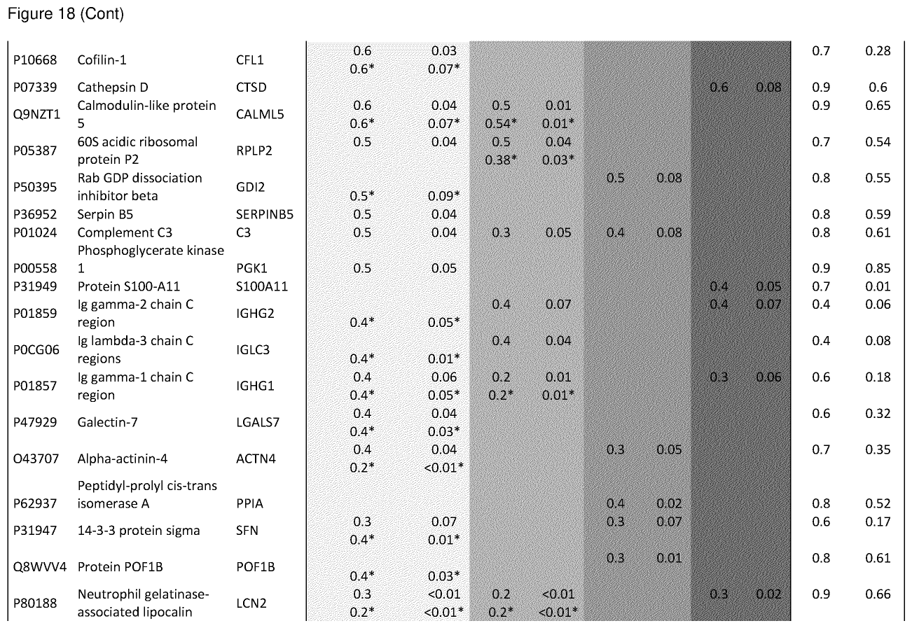

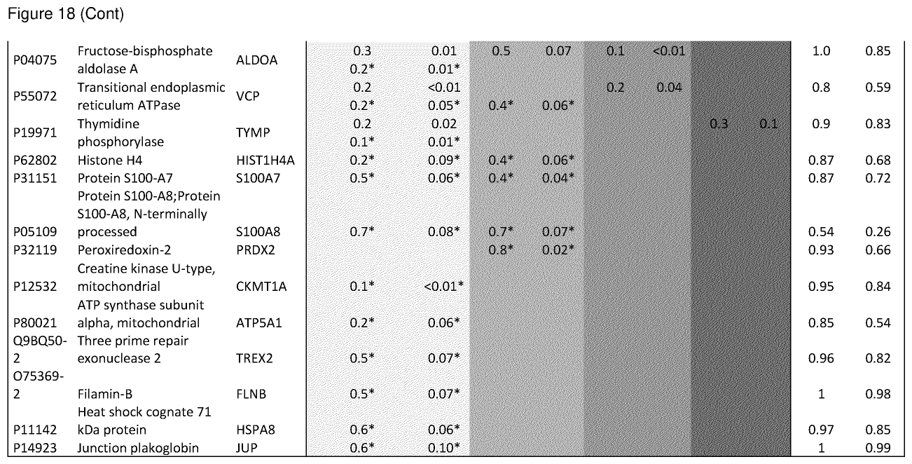

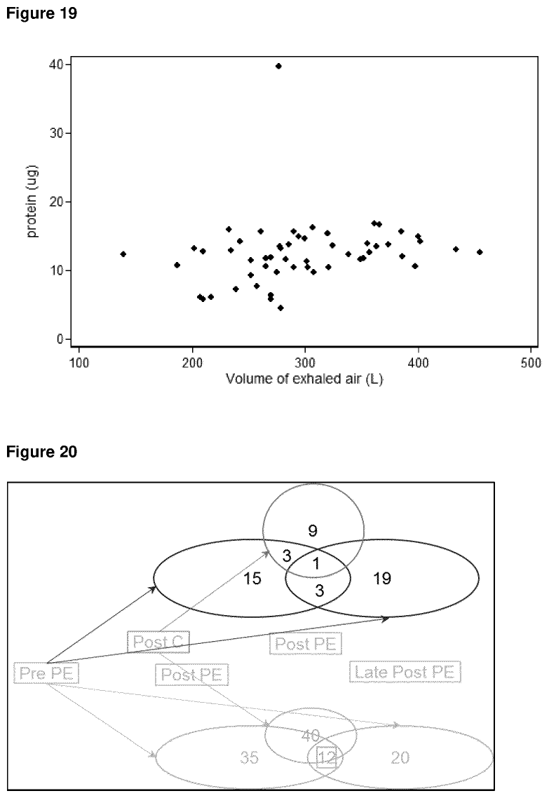

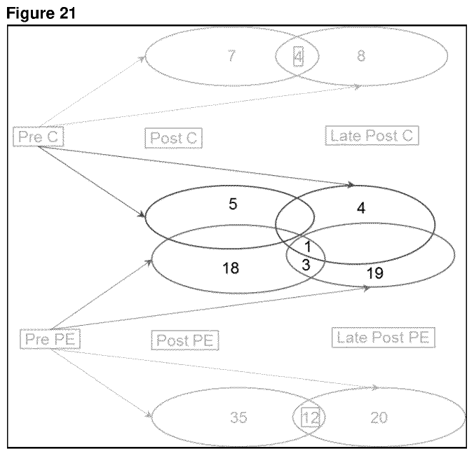

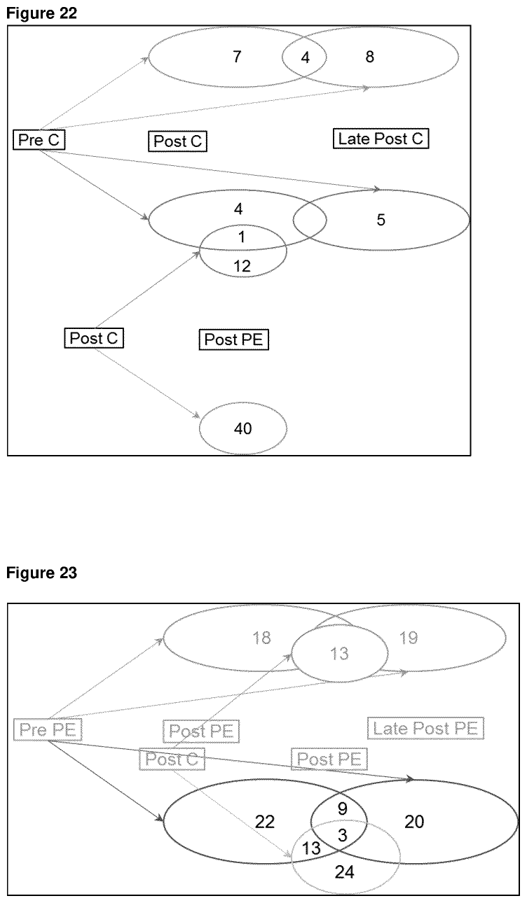

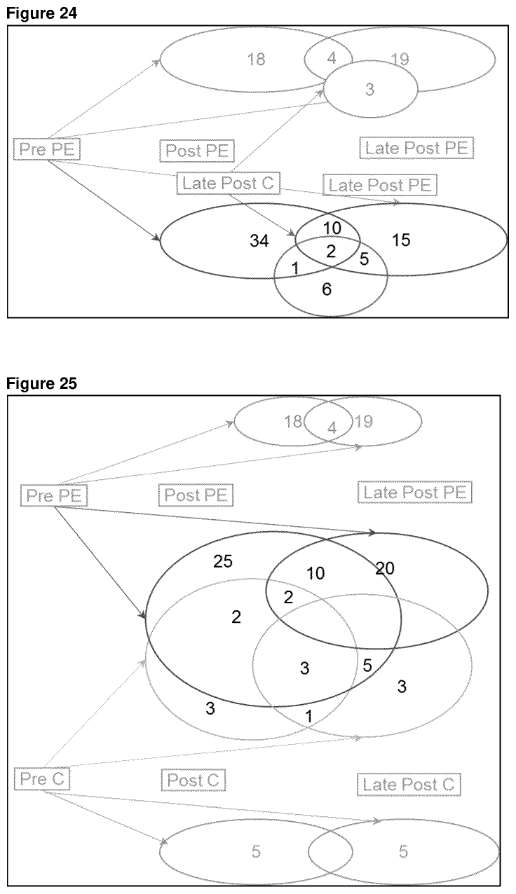

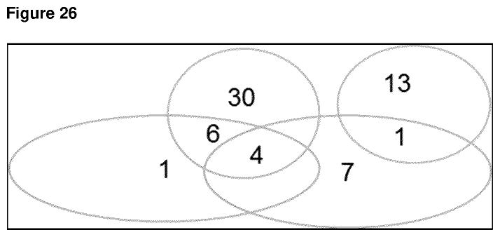

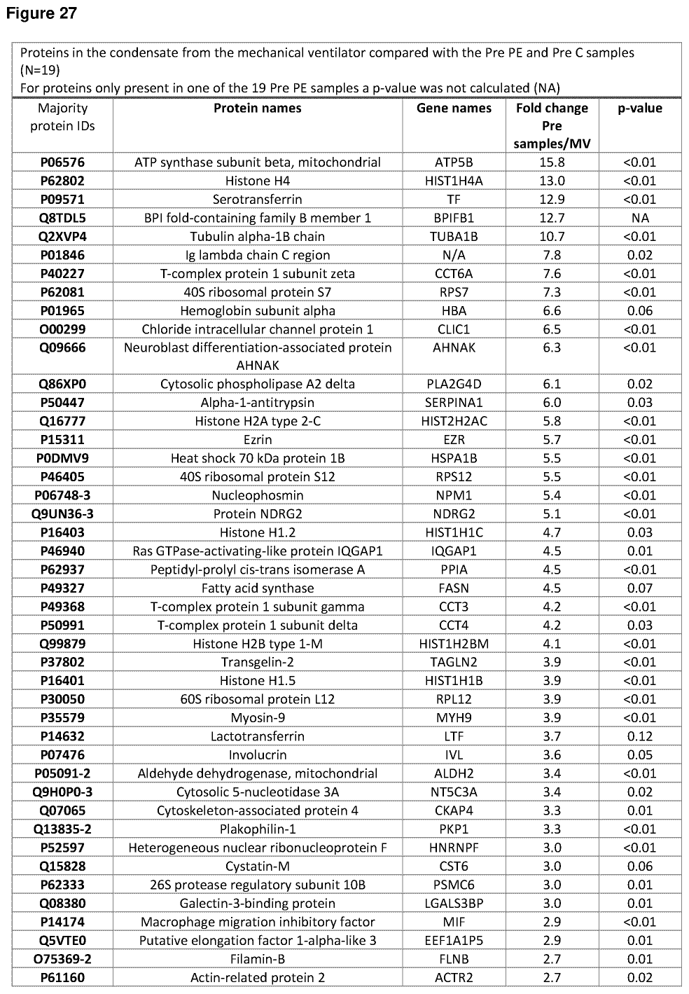

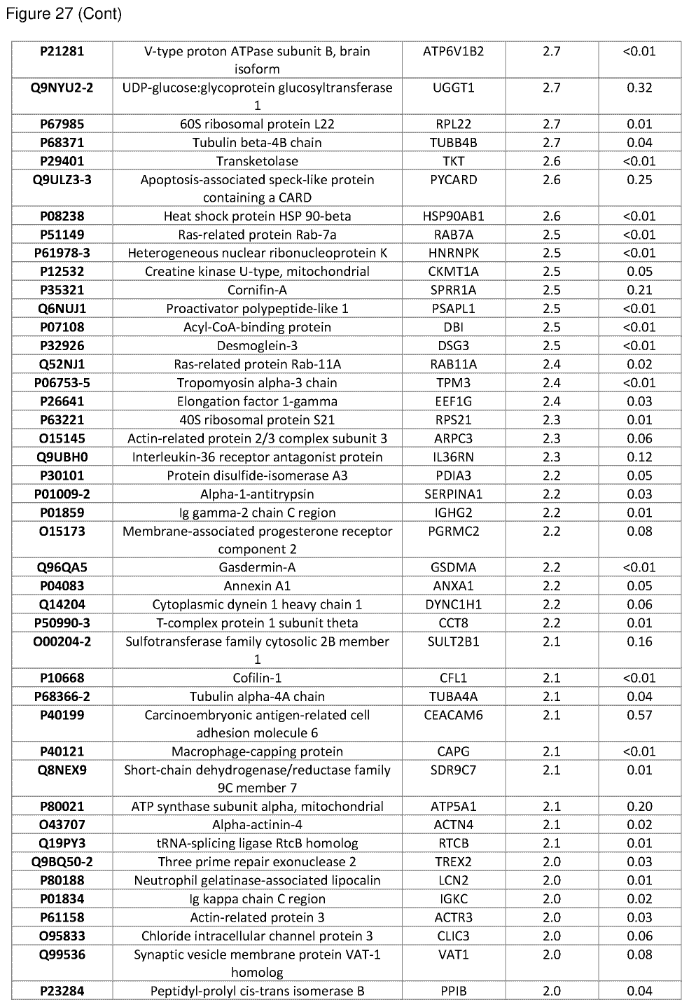

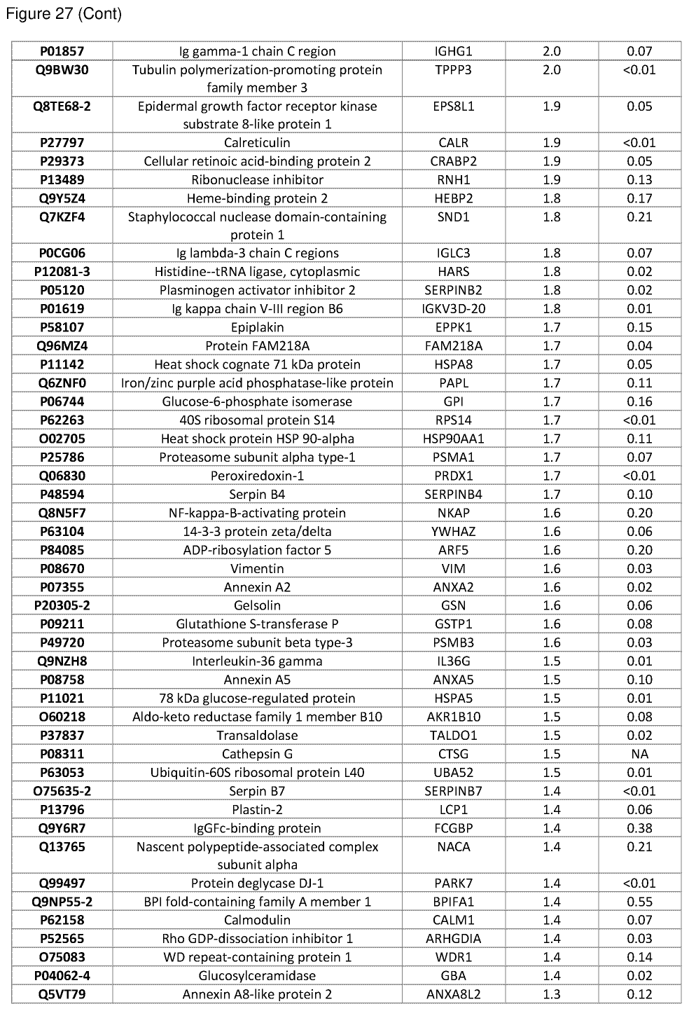

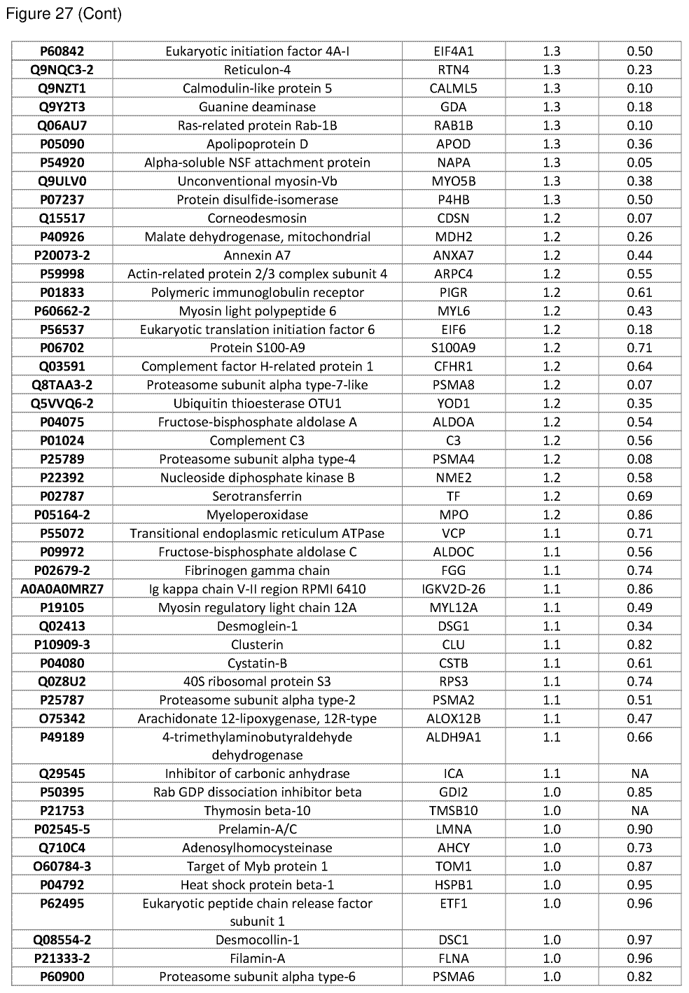

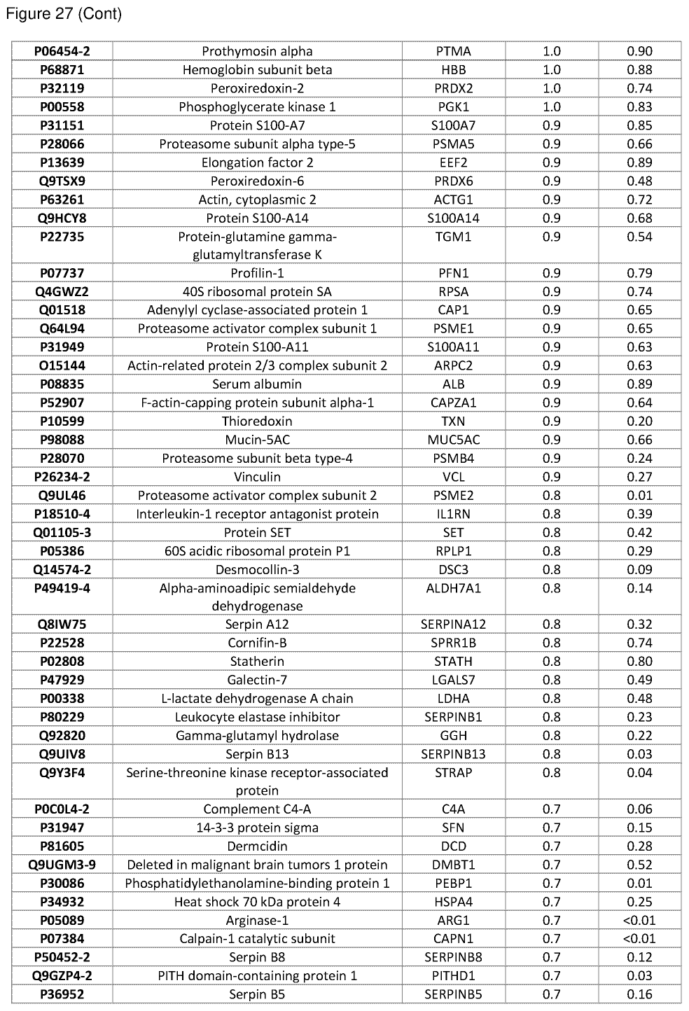

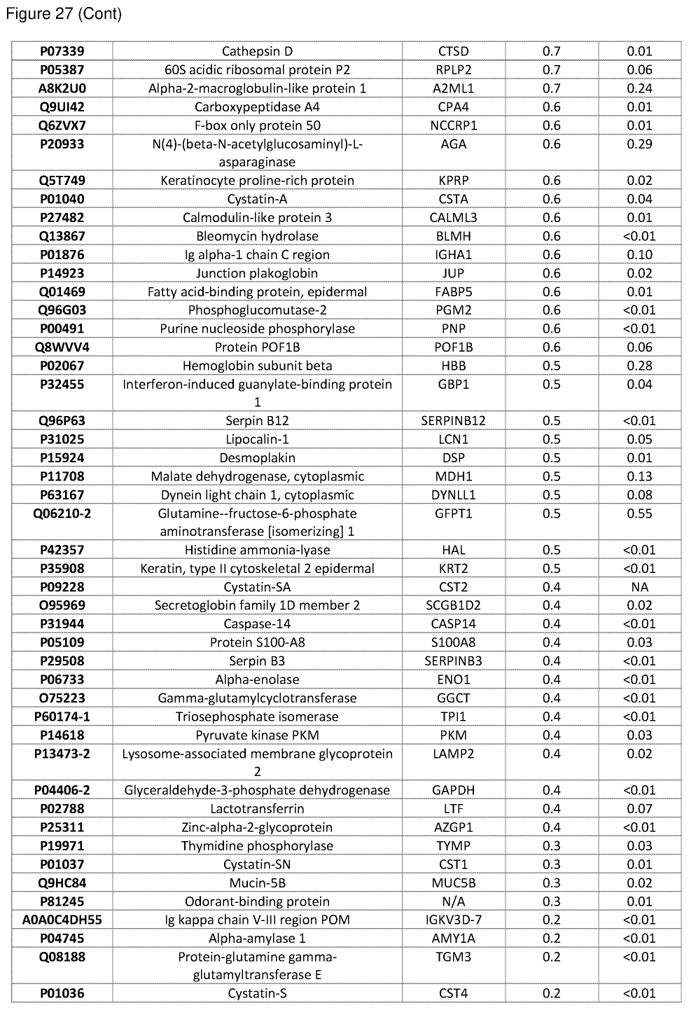

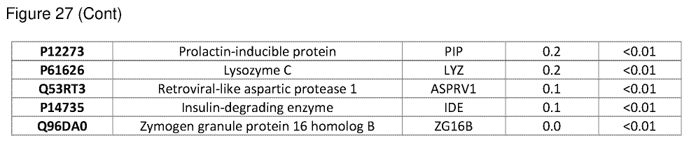

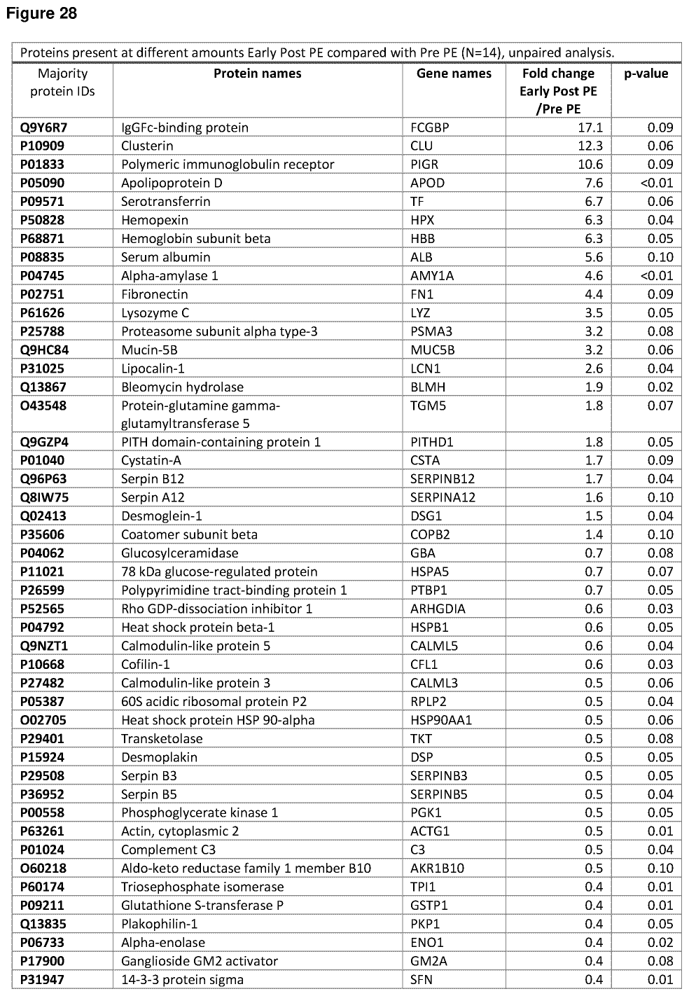

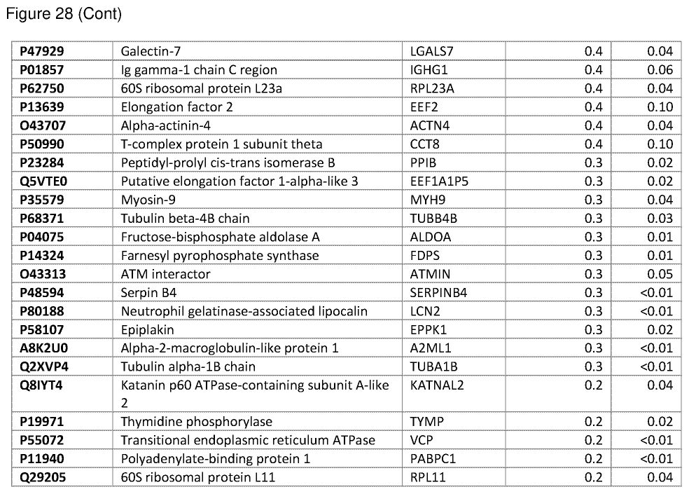

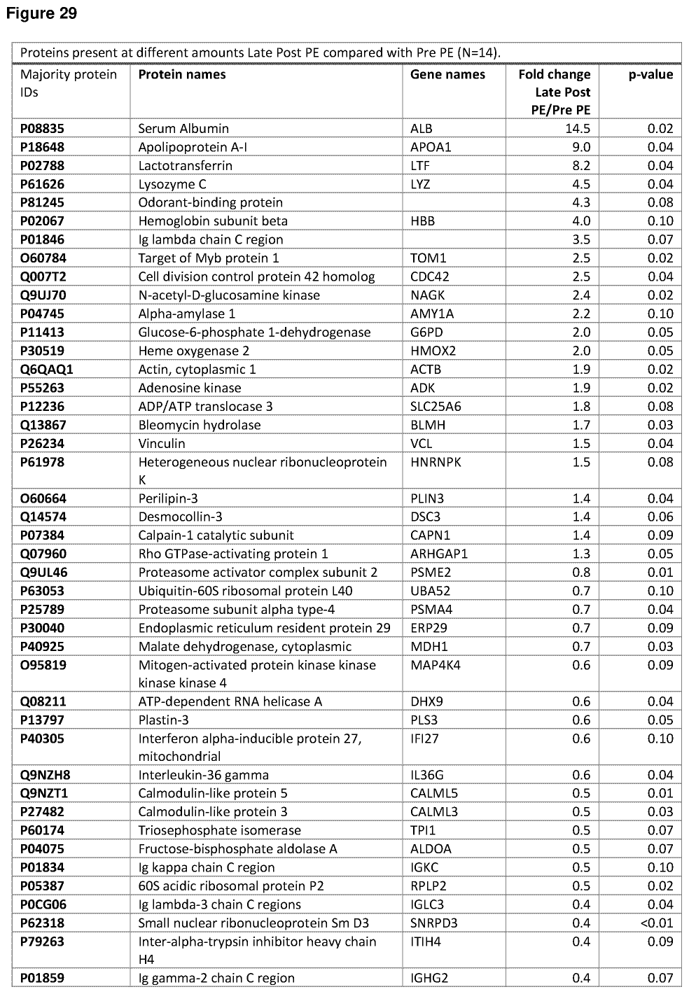

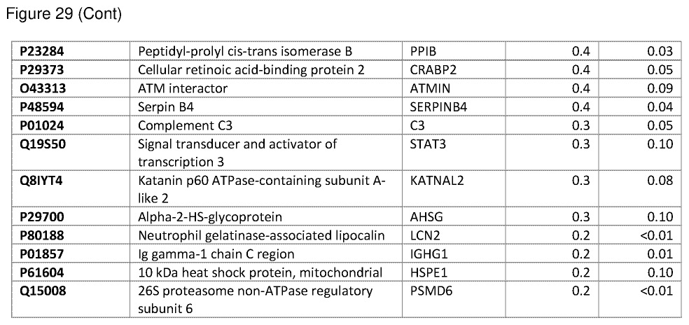

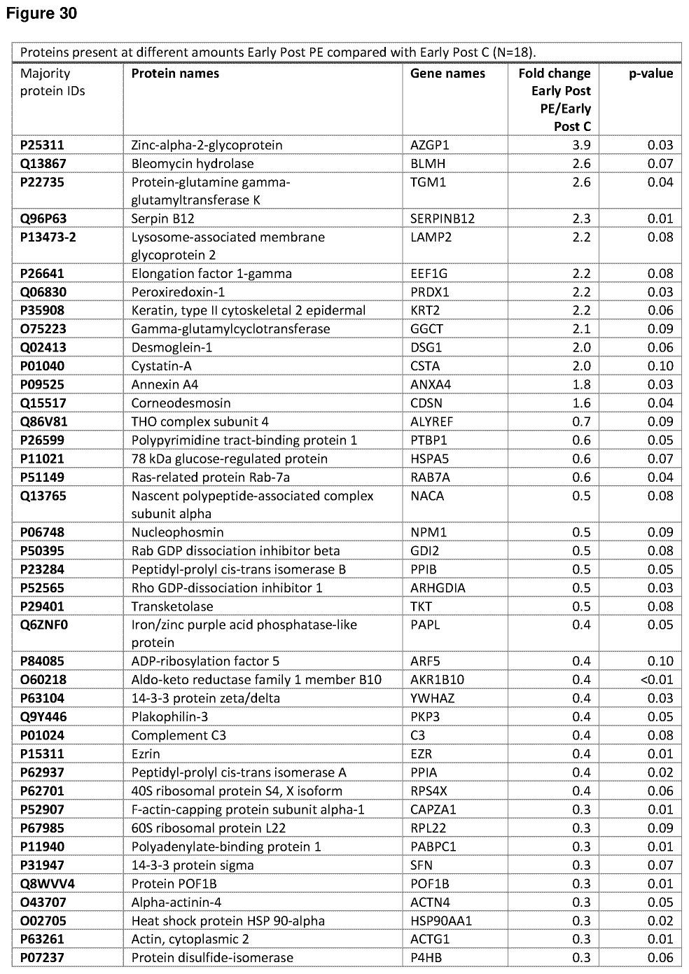

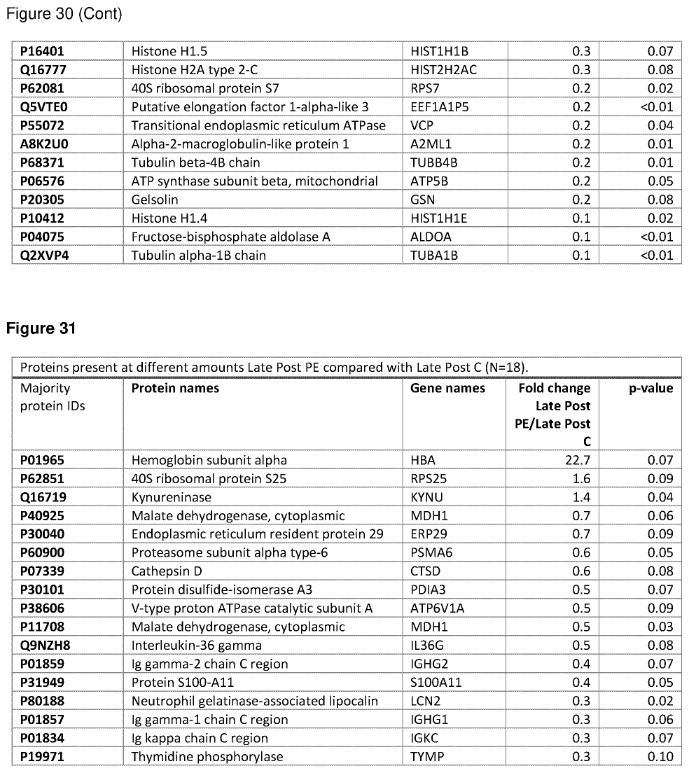

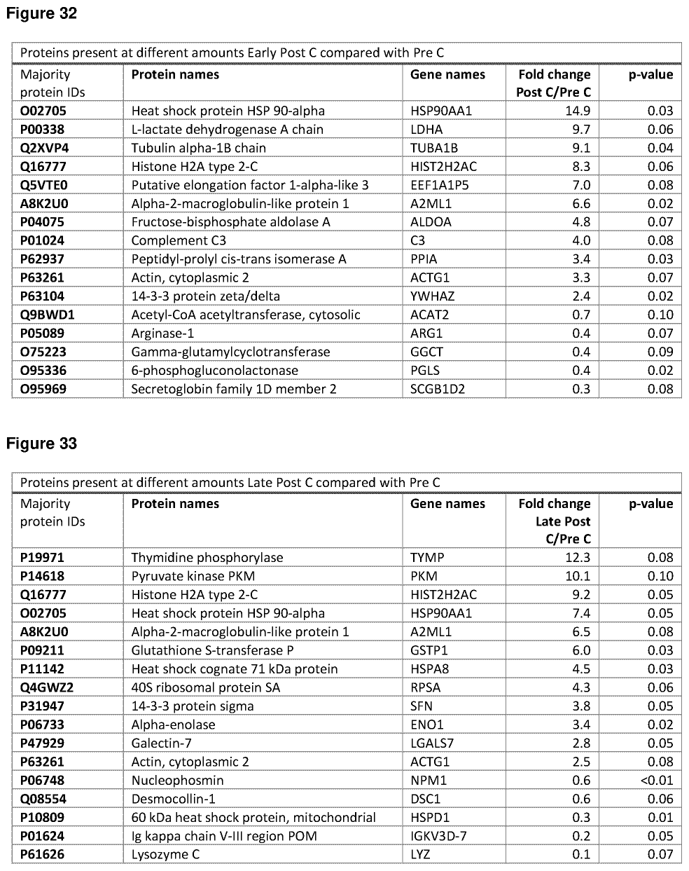

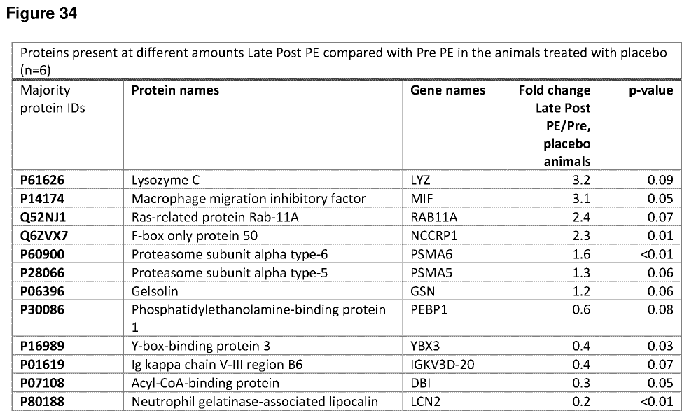

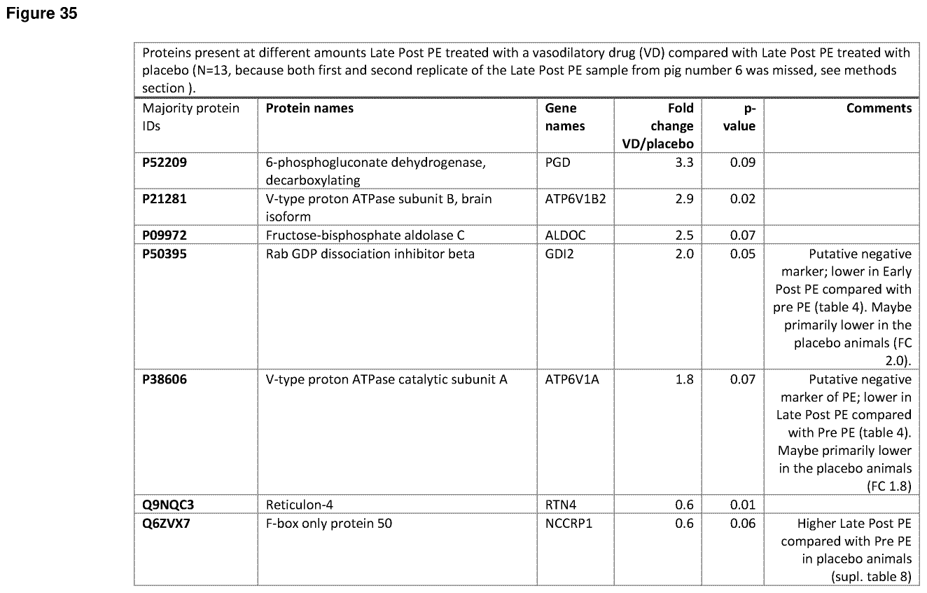

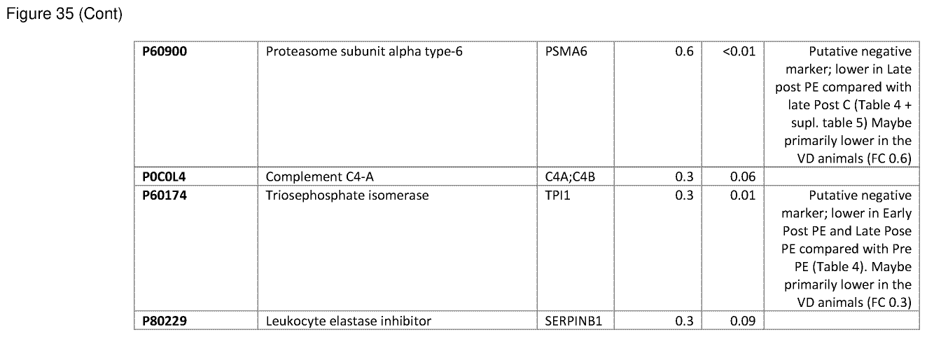

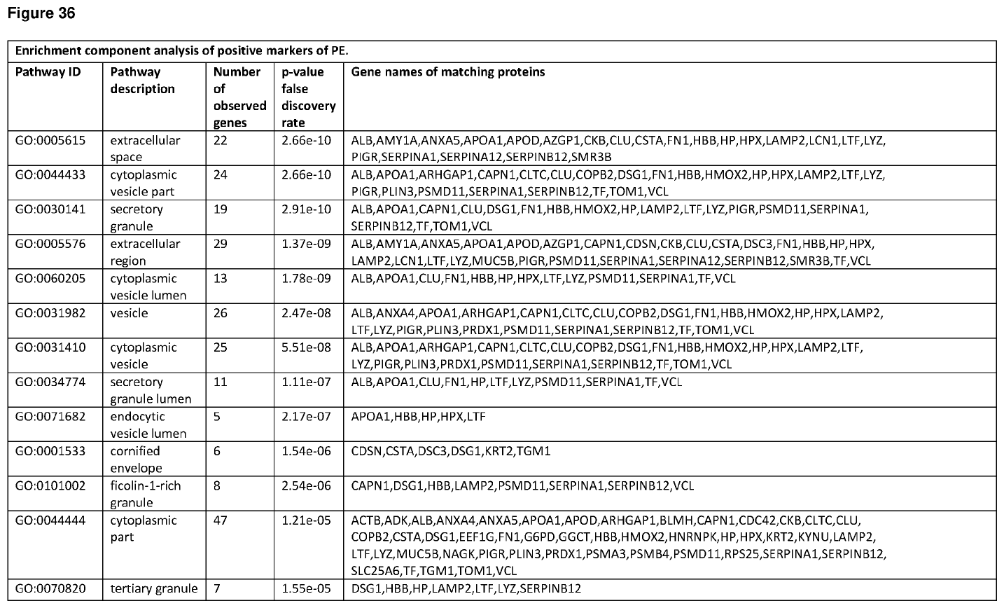

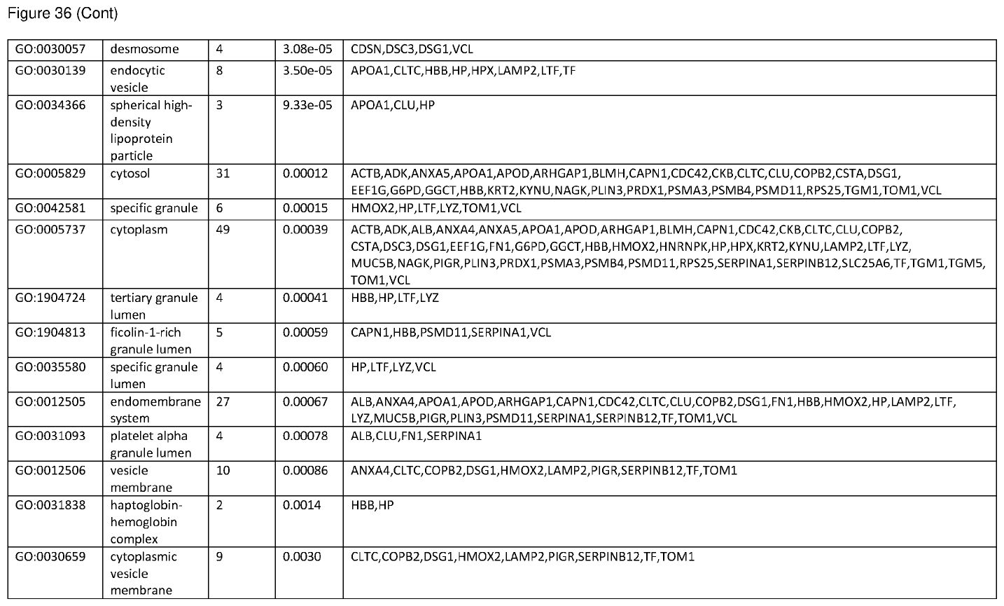

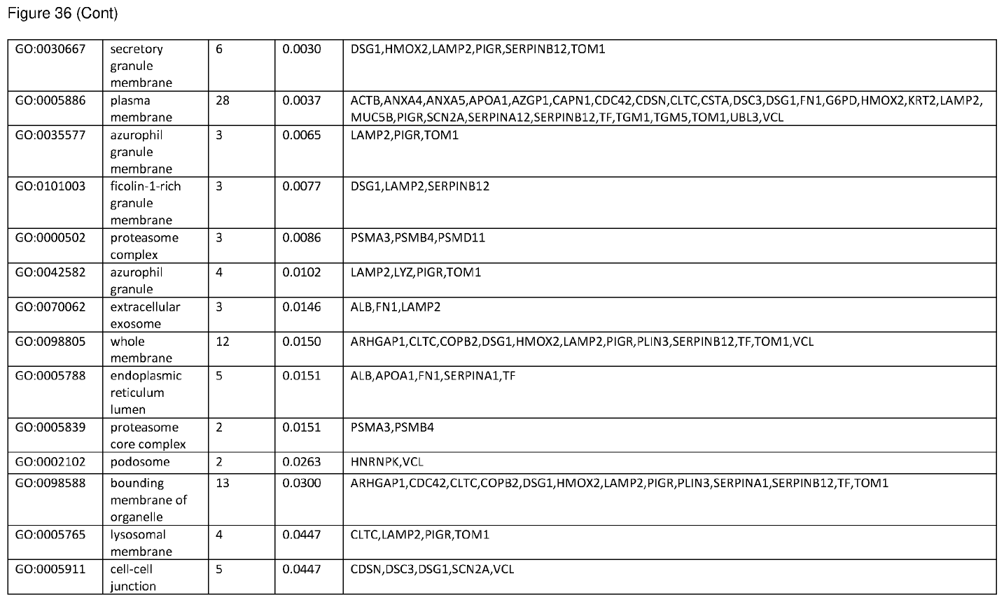

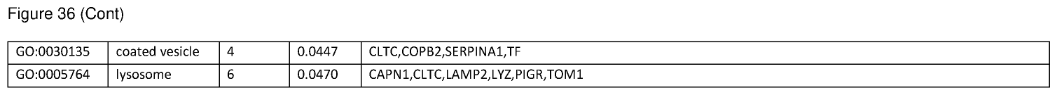

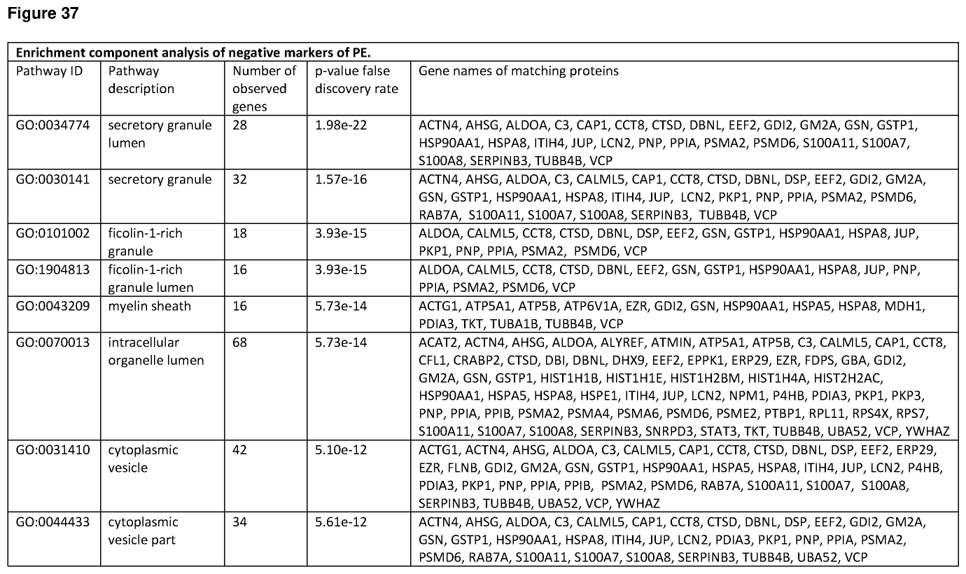

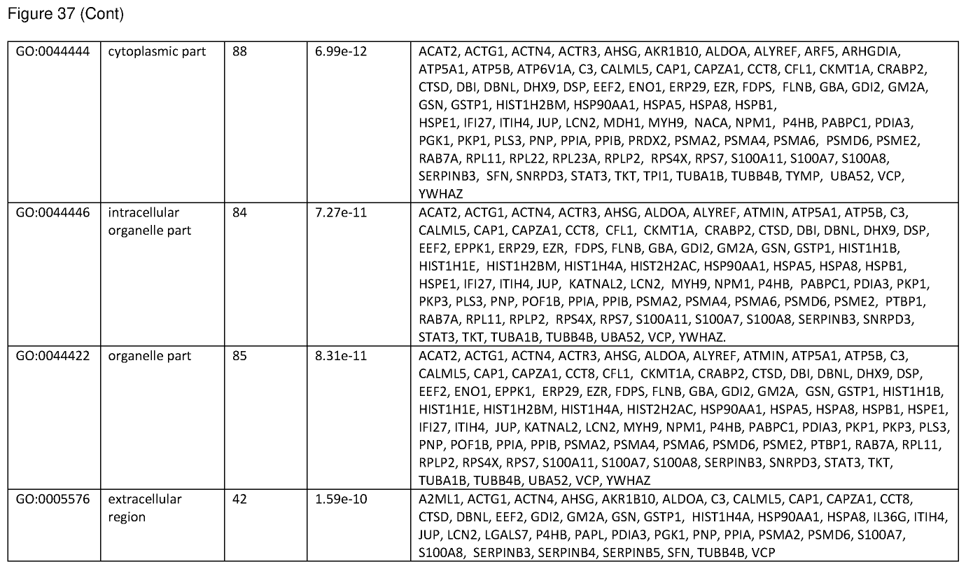

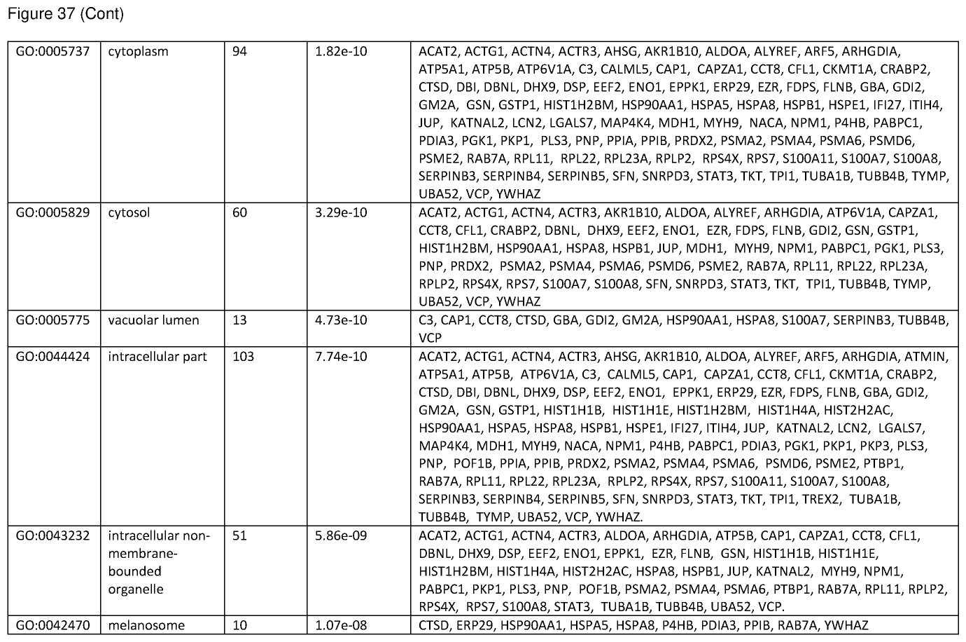

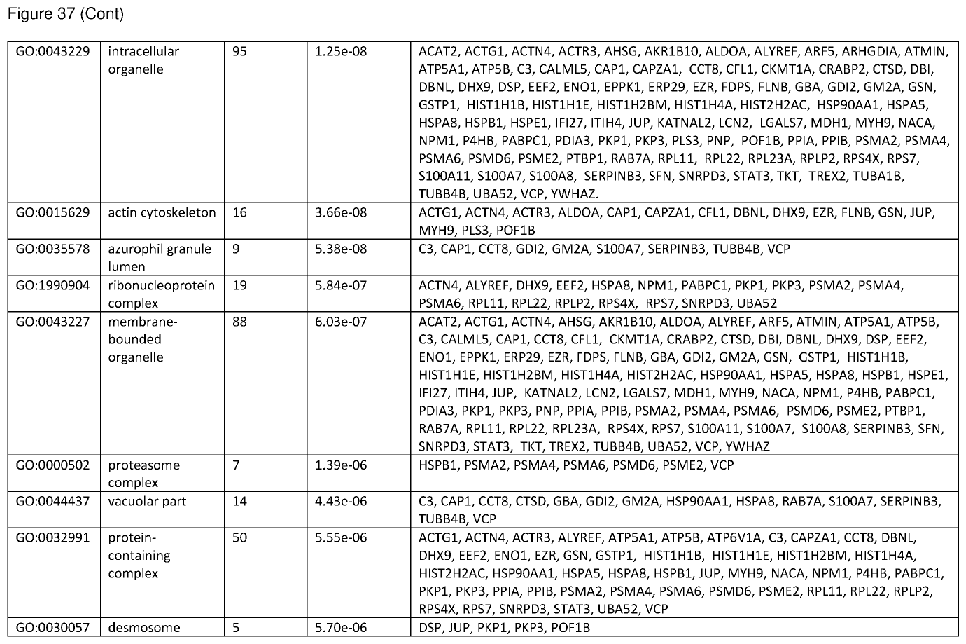

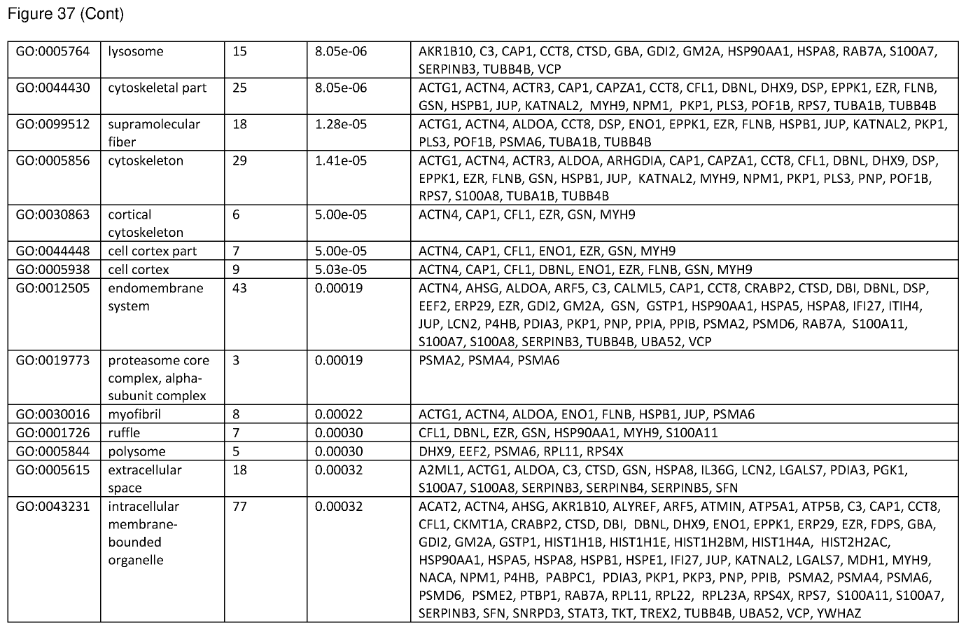

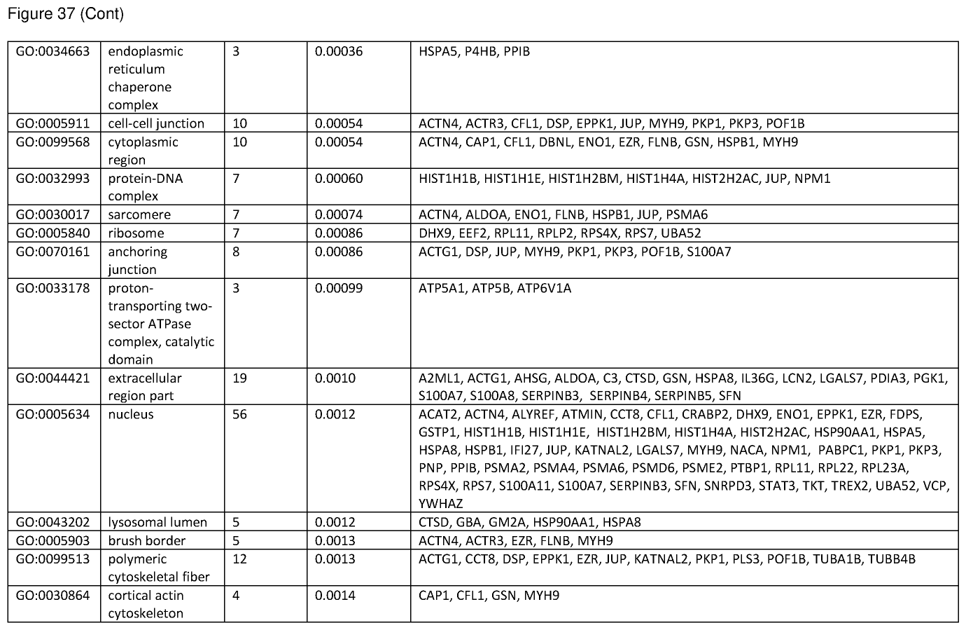

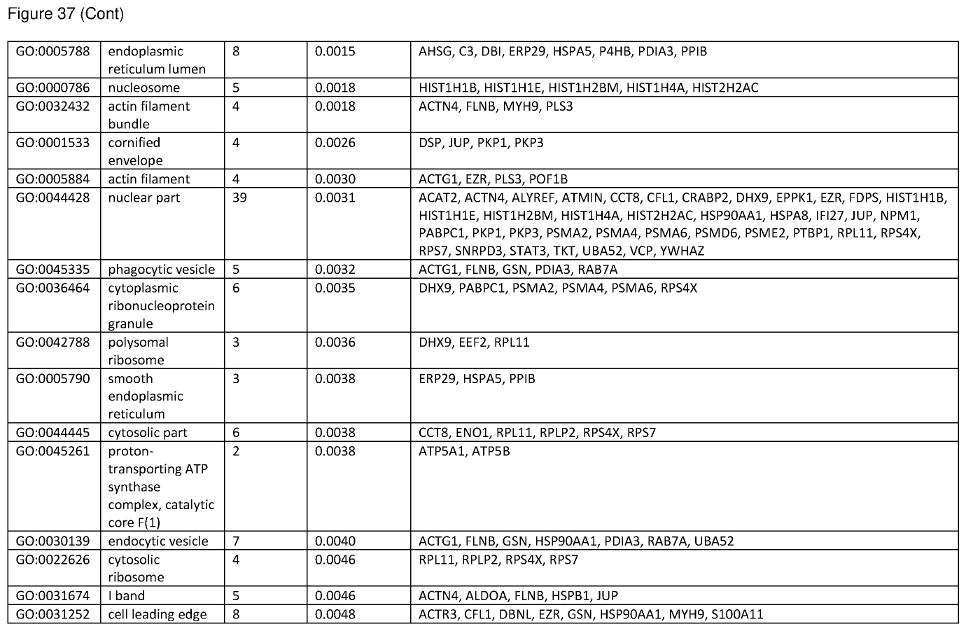

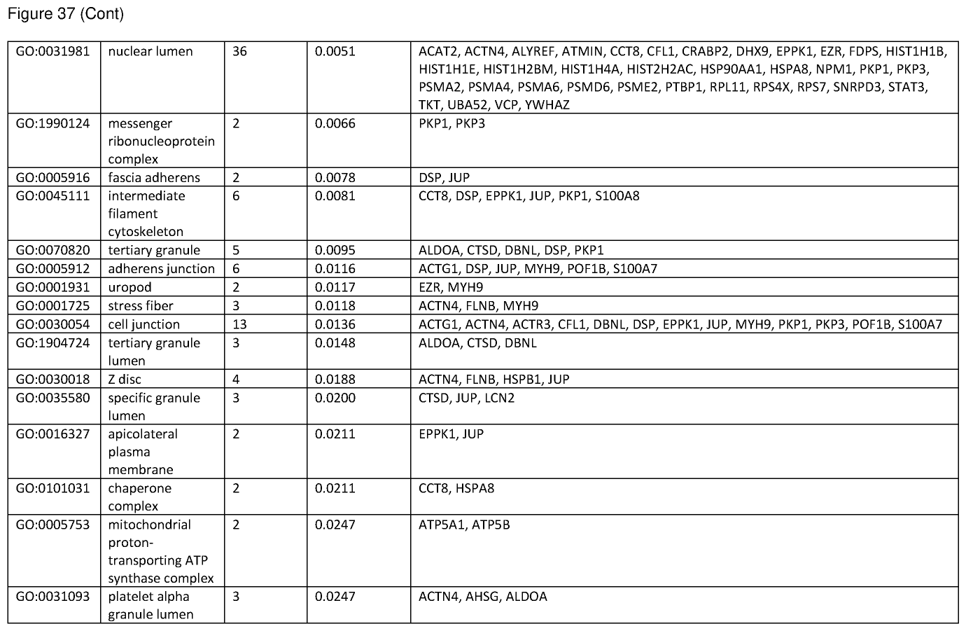

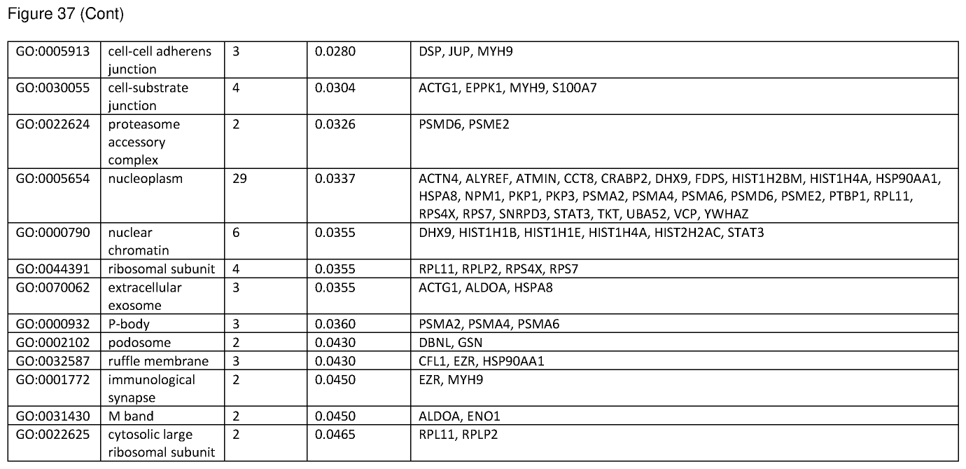

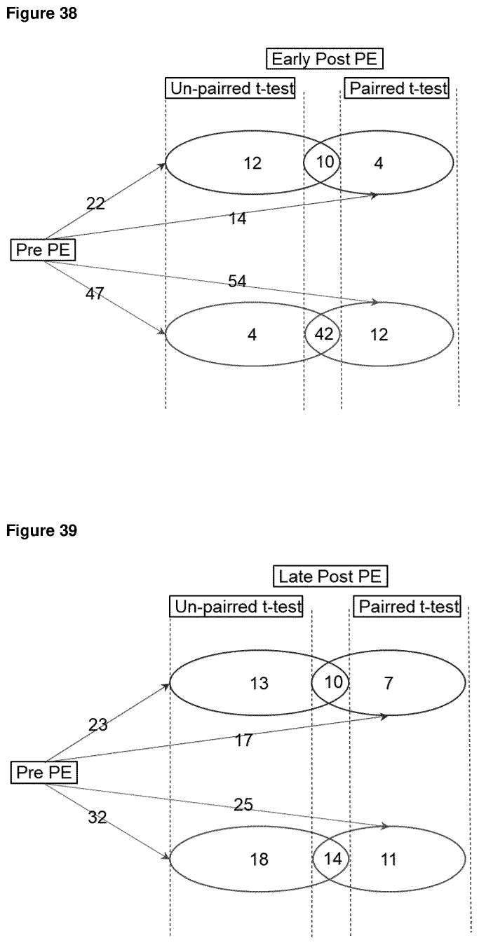

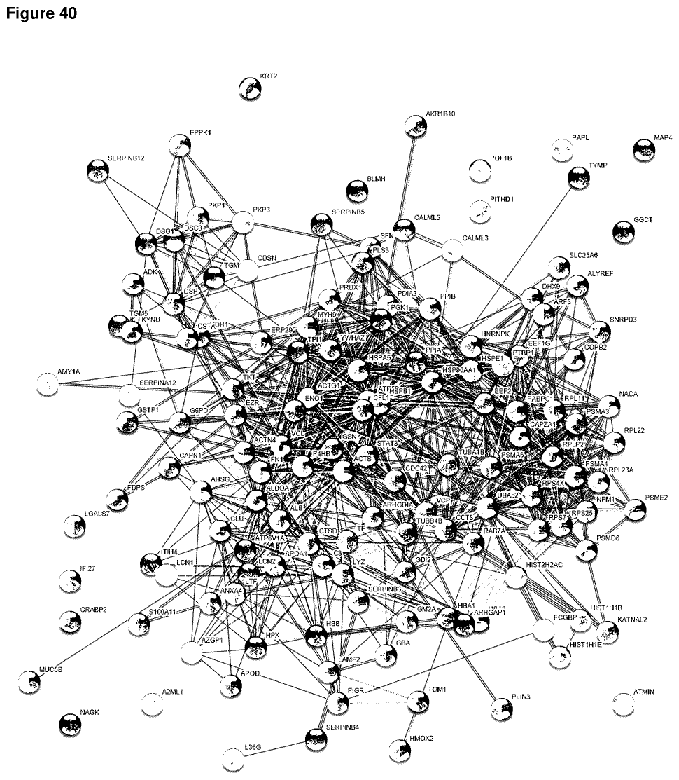

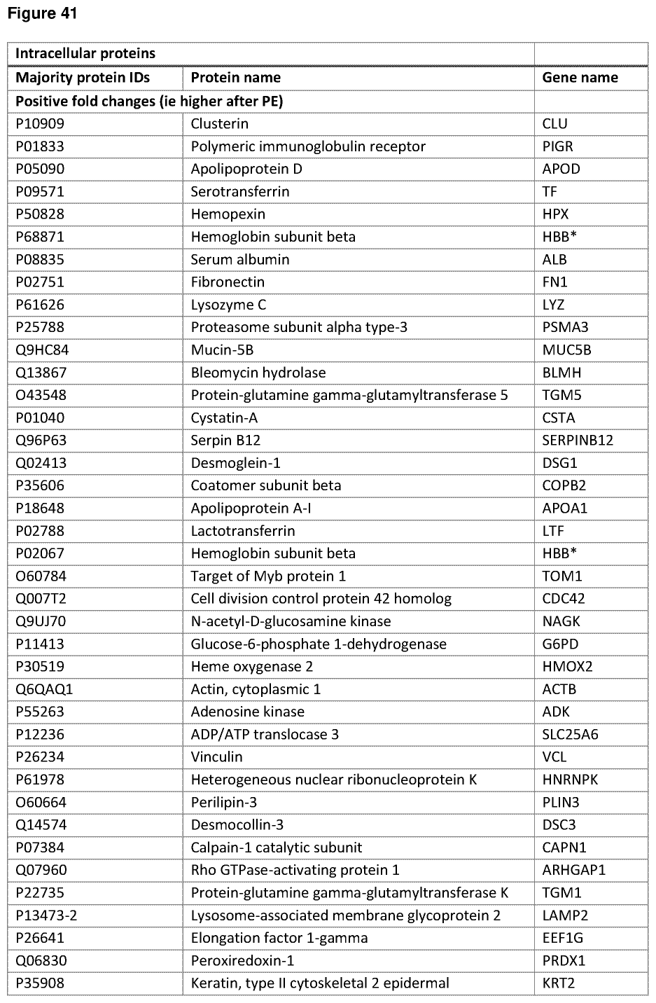

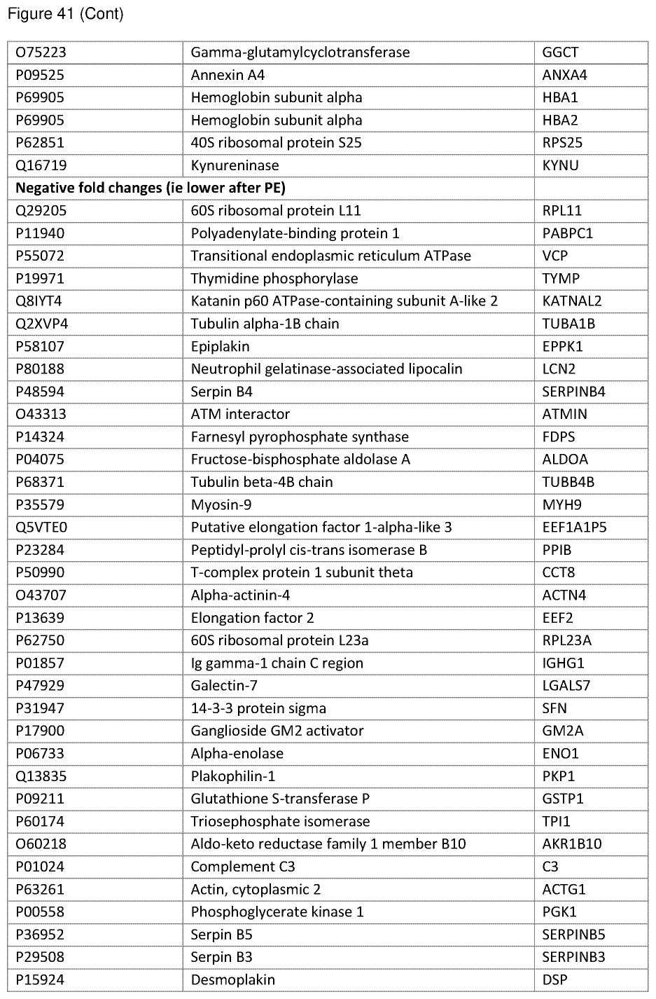

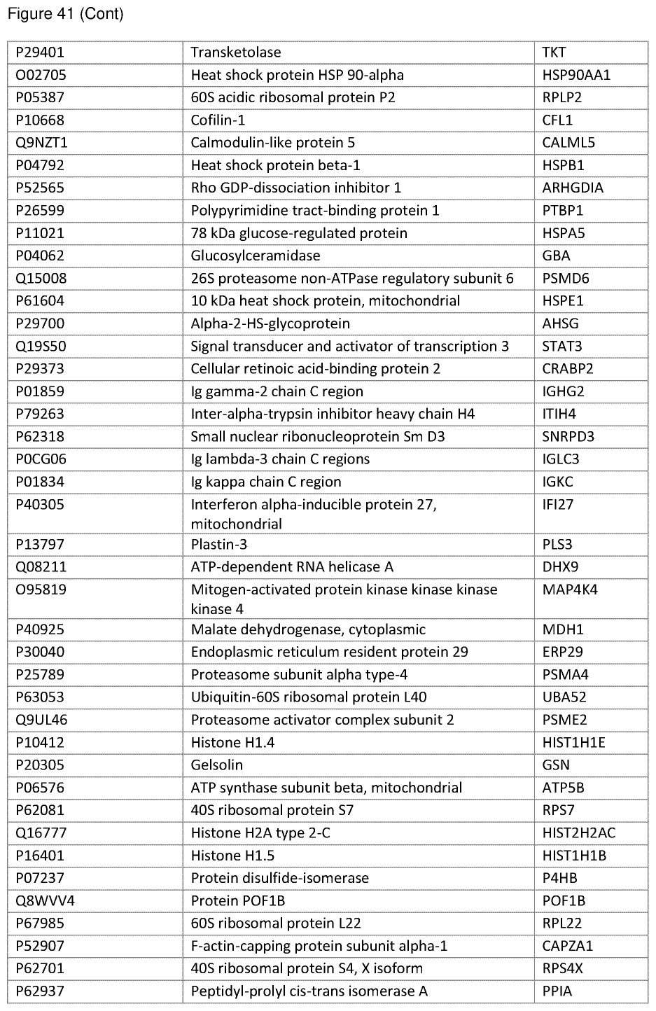

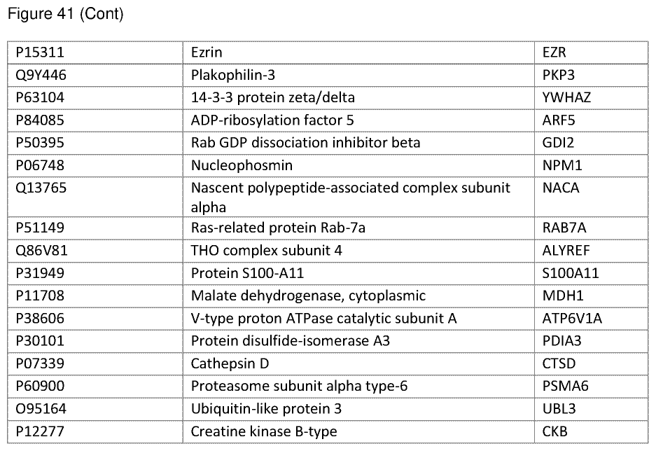

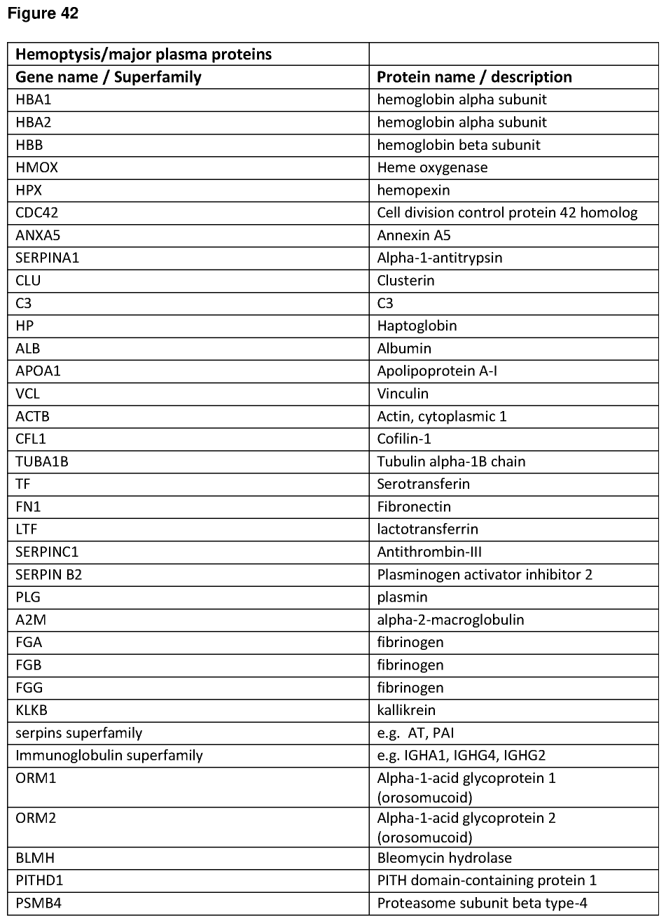

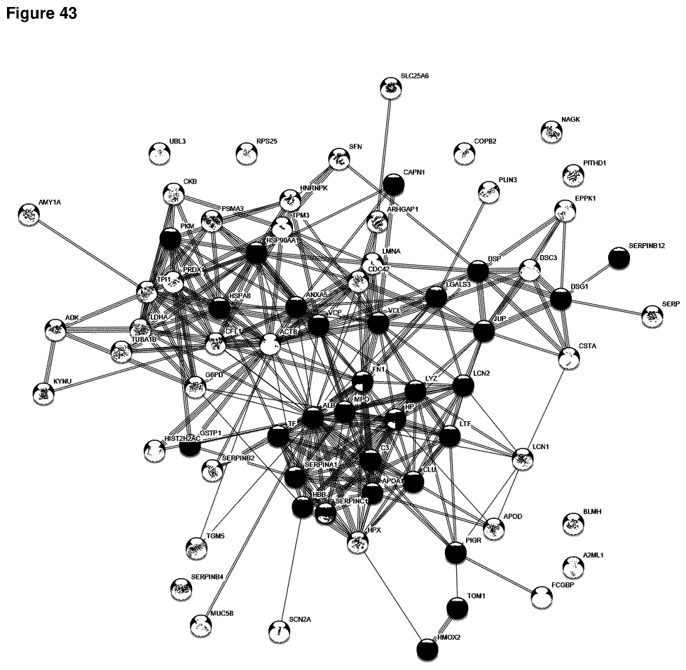

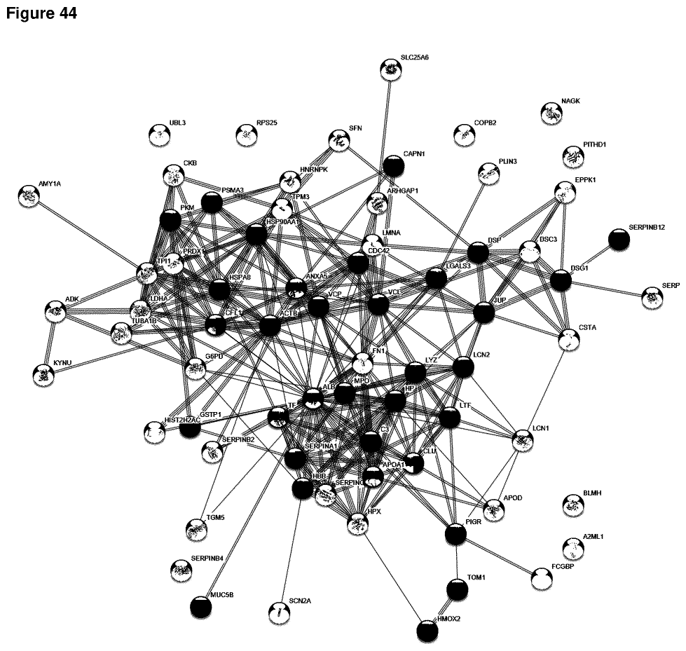

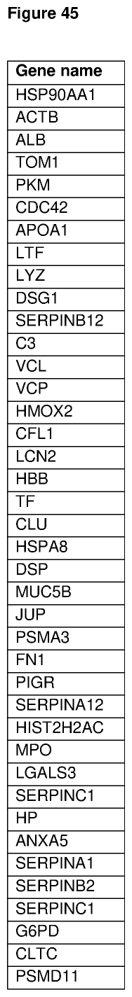

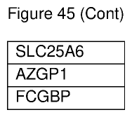

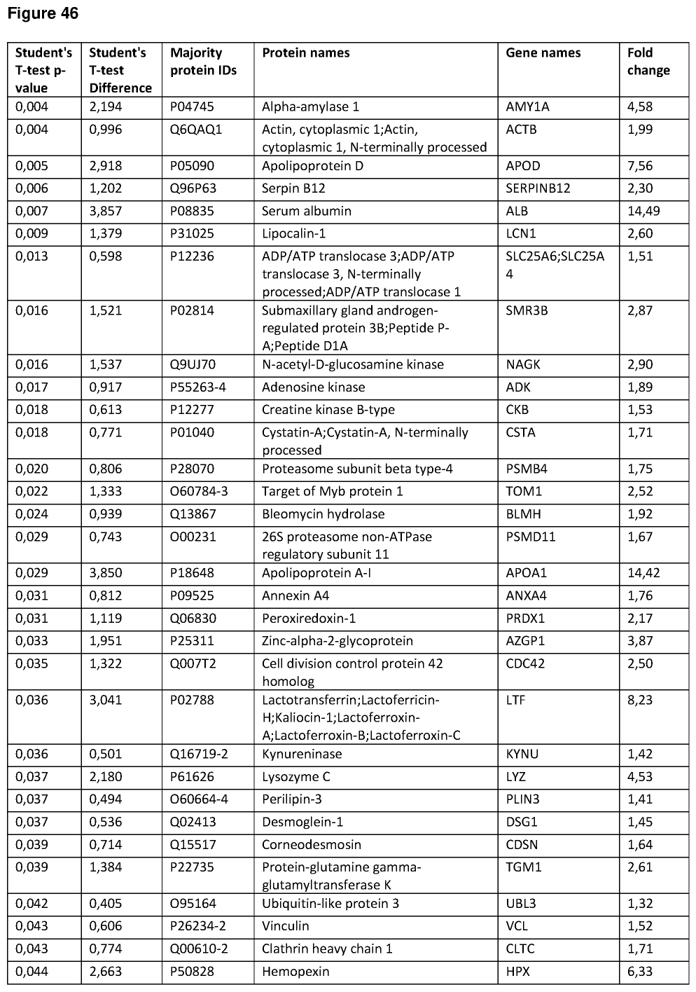

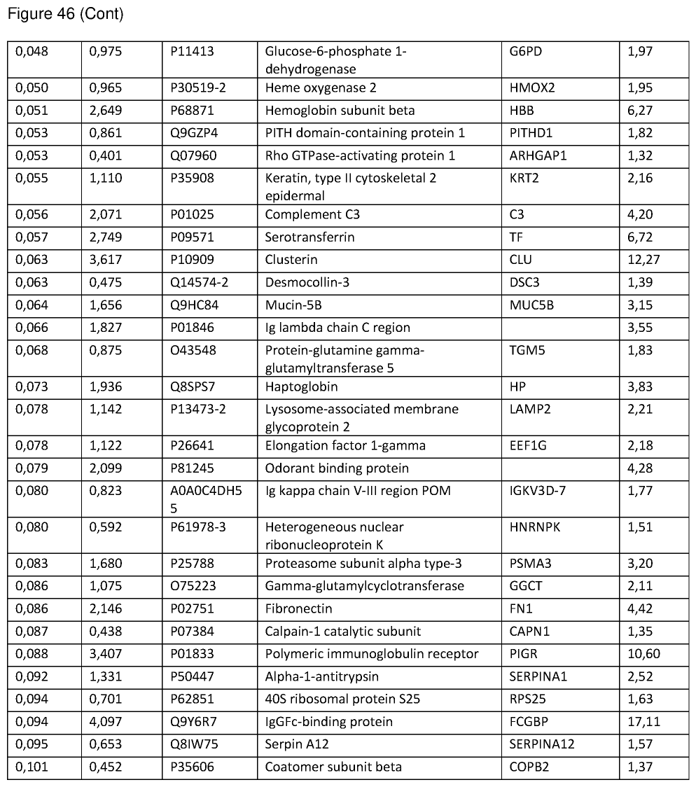

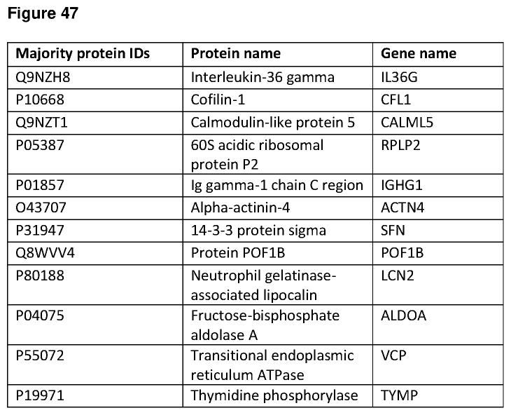

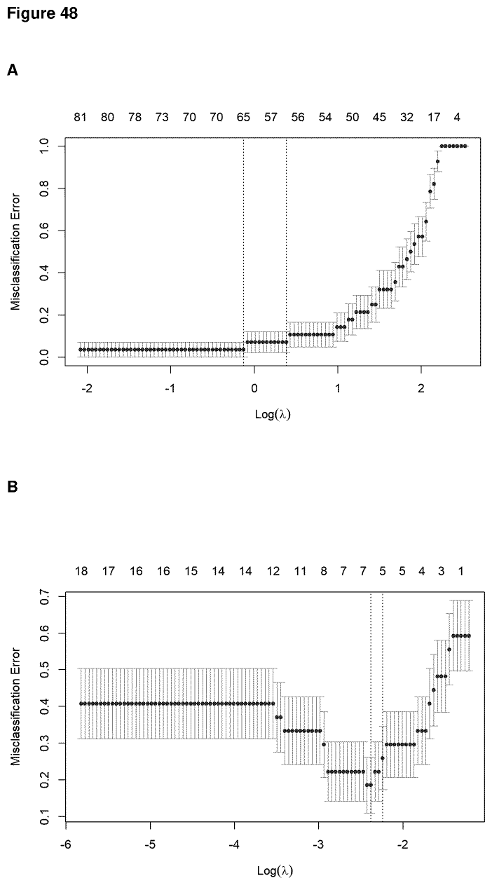

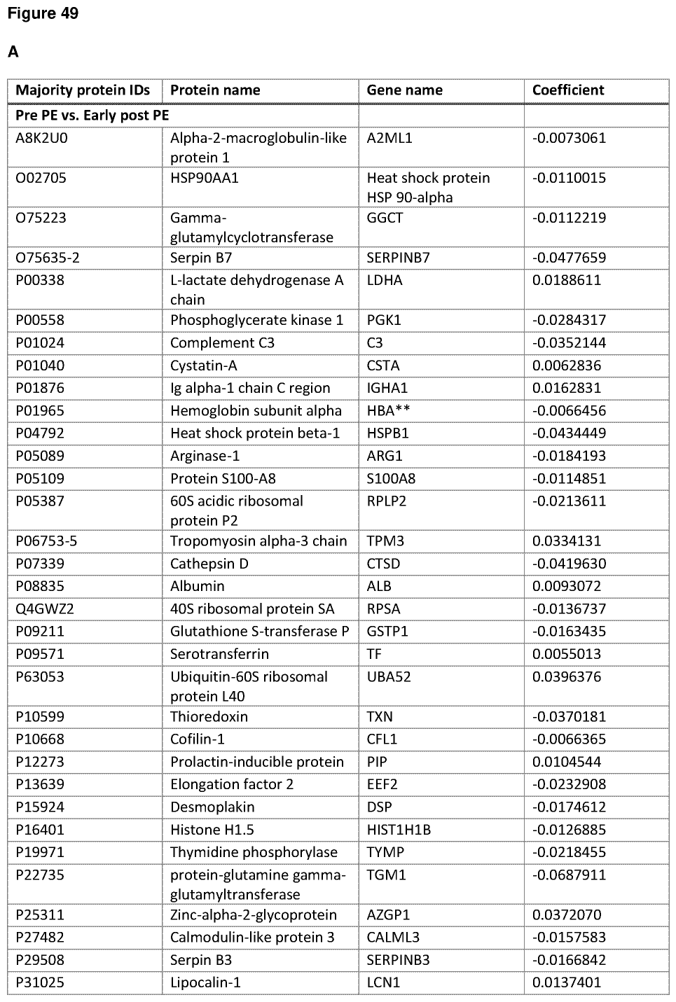

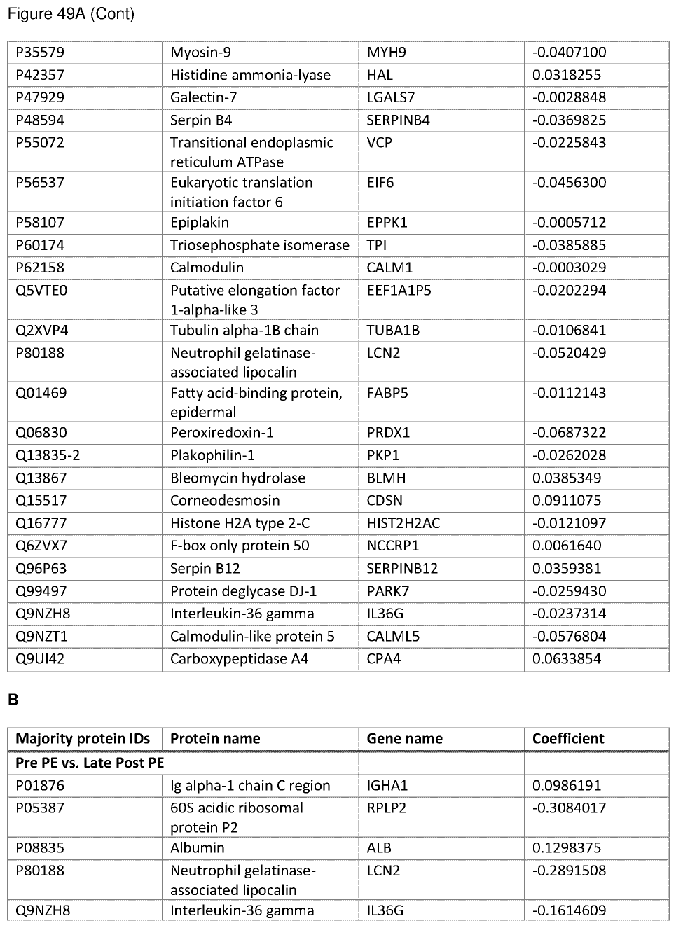

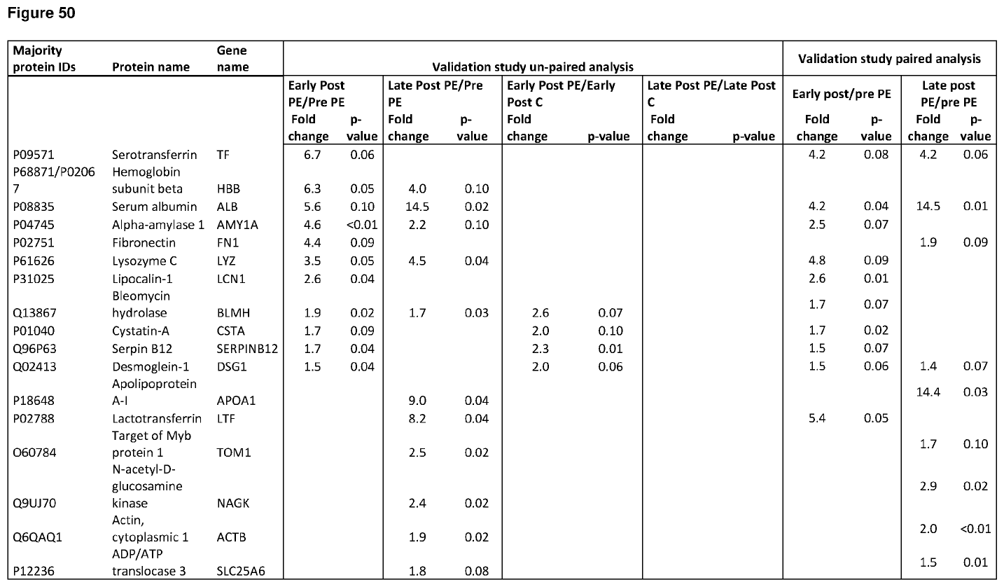

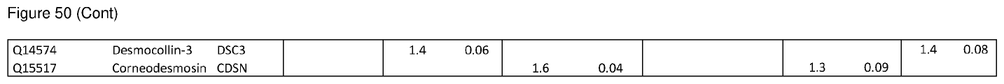

In total 62 proteins fulfilled at least one of these criteria (FIGS. 15 and 17). Two of the proteins (major protein IDs P01846 and P81245) were excluded, as they did not have a Homo sapiens counterpart (FIG. 15). Interestingly, some of the proteins from the unpaired analysis were upregulated both in comparisons between Early Post PE and/or Late Post PE with Pre PE (upper circles of FIG. 5), and also in comparisons between Early Post PE and Post C (upper circle of FIG. 6). These overlapping proteins are depicted in FIG. 9 and are further elaborated in FIG. 18. Also, a few proteins were found to be increased in PE samples and at the same time decreased in control samples (FIGS. 19 and 20). The positive markers of PE are listed in table 1, 37% of them were not identified in the condensate from the mechanical ventilator (FIG. 27). Bioinformatics analysis showed that the proteins were related to a significantly higher extend than expected for a random set of proteins (FIGS. 10 and 36). A large part of the proteins were extracellular proteins.

Negative Markers

The criteria for selection as “negative marker” for PE (i.e. a protein that would be present at very low amounts (if present at all) in case of PE and possibly present in higher amounts in non-PE cases) were:
I. Proteins significantly downregulated in the Post PE or Late Post PE samples compared with Pre PE (represented in the lower circles of FIG. 5) and not significantly downregulated in the Post C or Late Post C compared with Pre C (not represented in the lower circles of FIG. 8).
II. Proteins significantly downregulated in Post PE compared with Post C (represented in the lower circle of FIG. 6) and not downregulated in Post C compared with Pre C (not represented in the lower circles of FIG. 8).
III. Proteins significantly downregulated in Late Post PE compared with Late Post C (represented in lower circle of FIG. 7) and not downregulated in Late Post C compared with Pre C (not represented in the lower circles of FIG. 8).
IV. Proteins significantly downregulated in the Early Post PE or Late Post PE compared with Pre PE in the paired analysis.

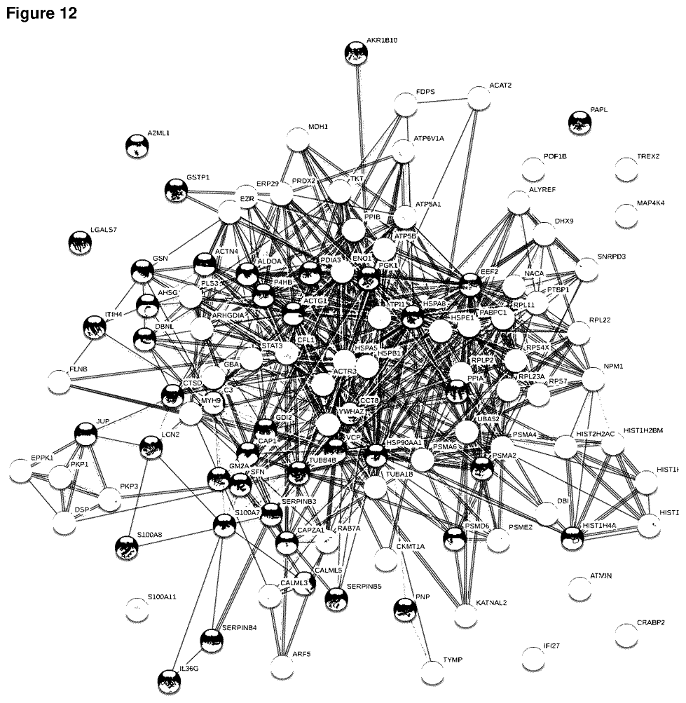

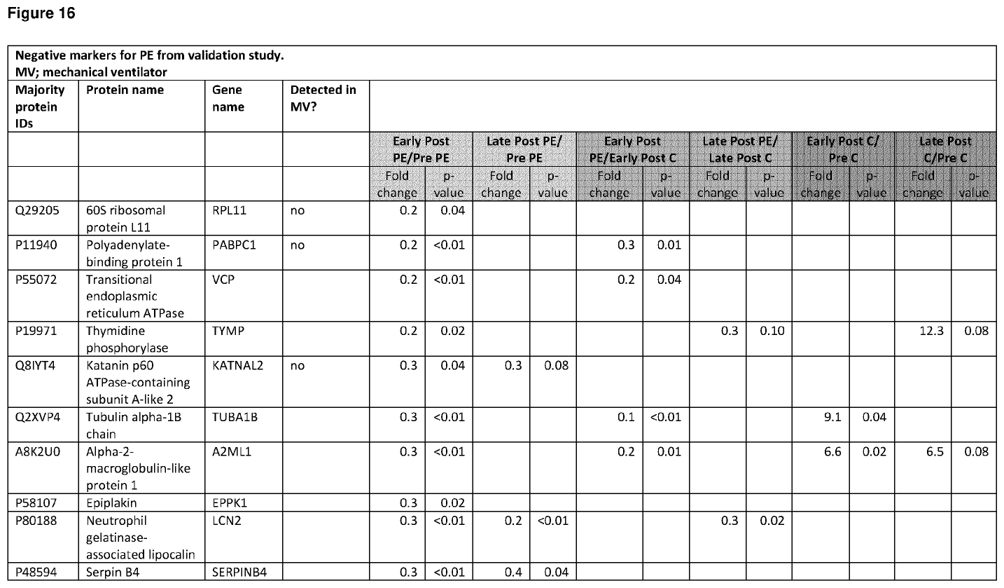

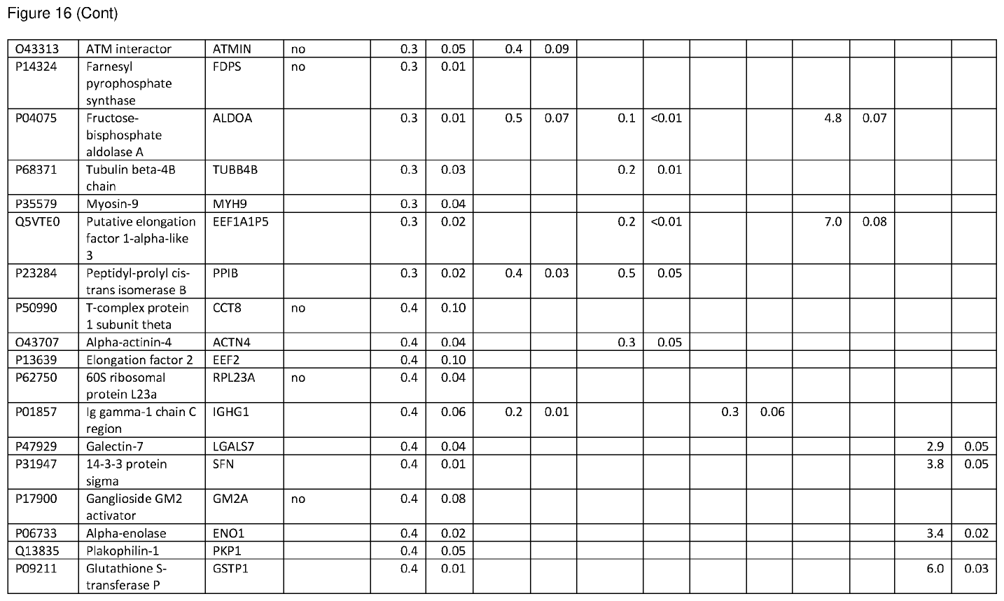

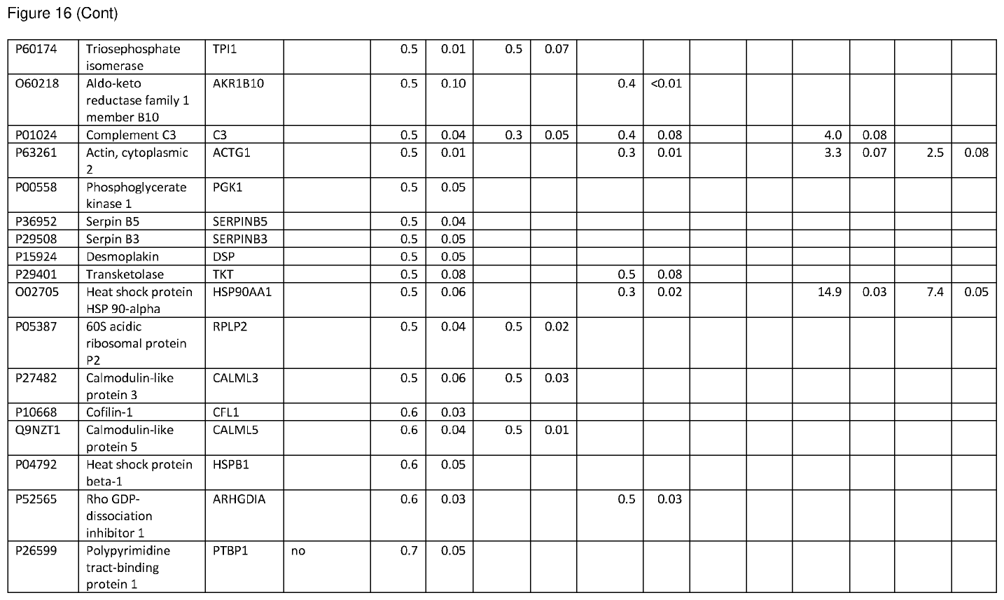

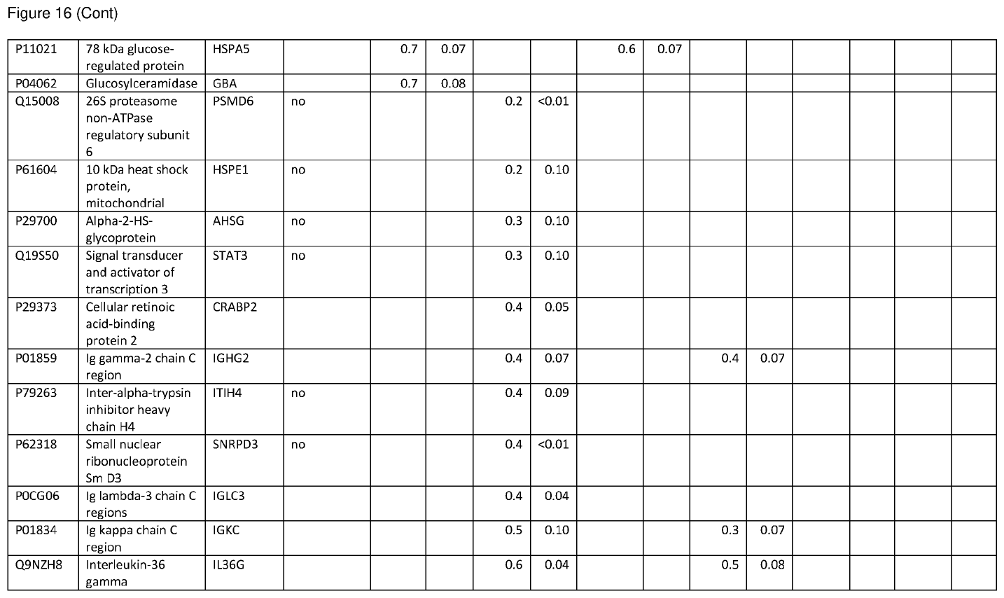

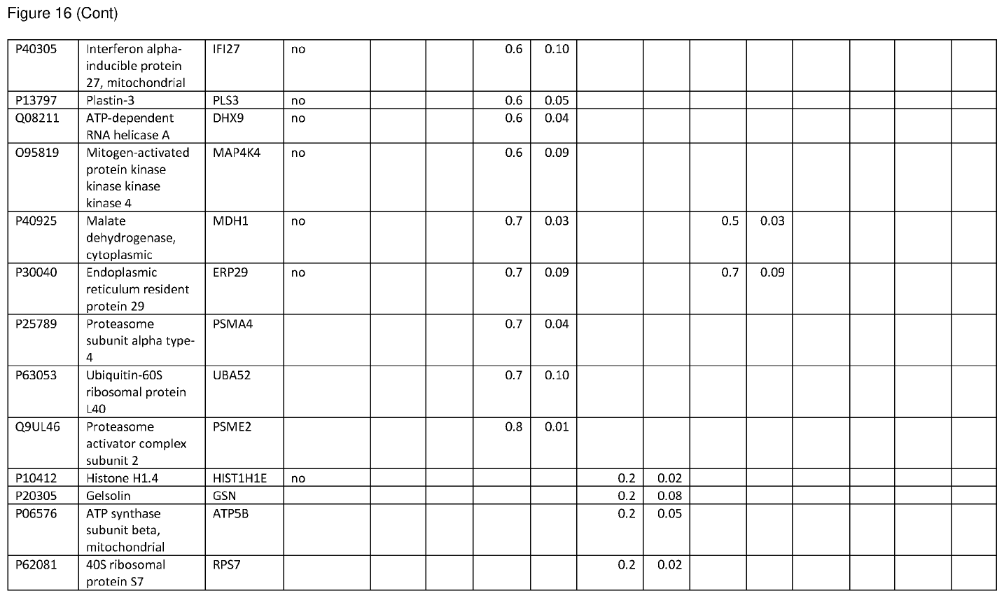

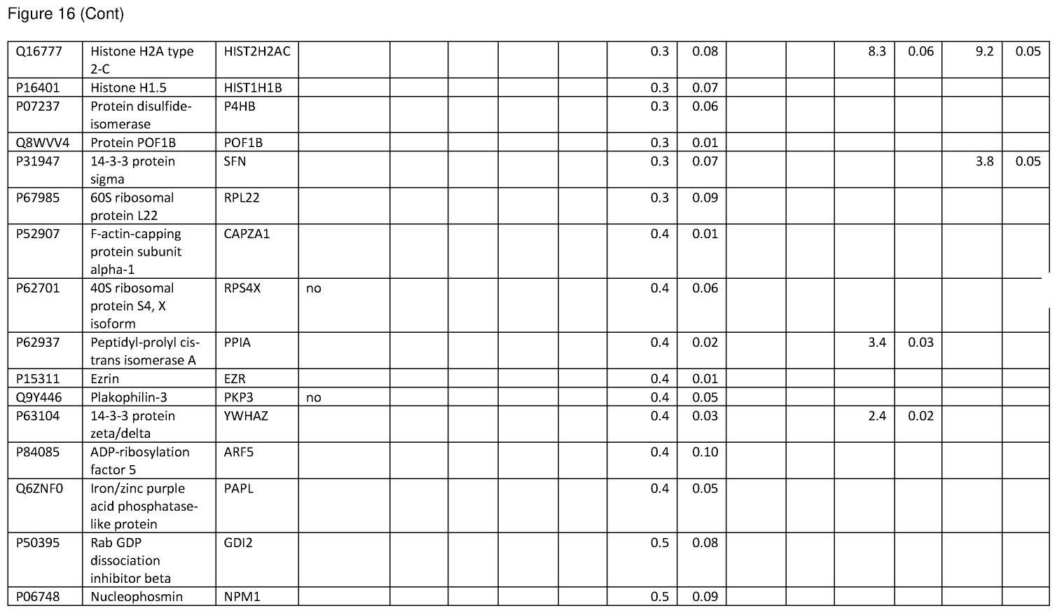

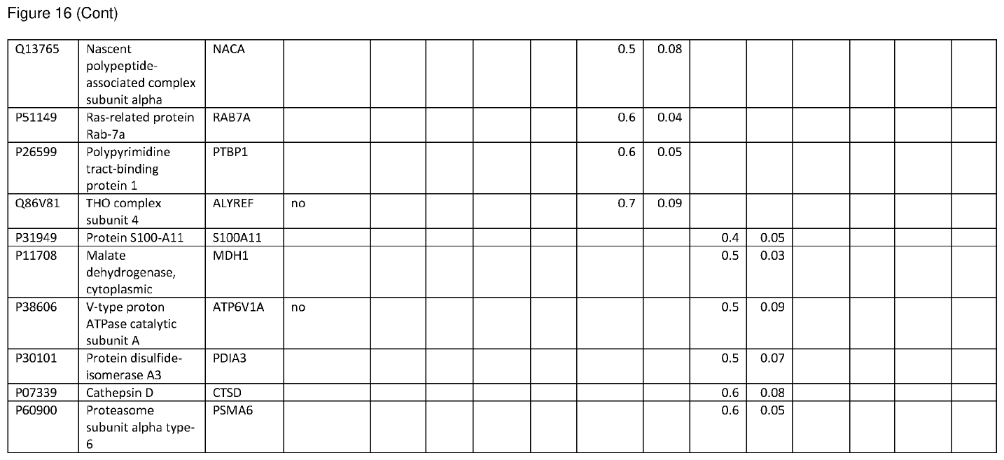

In total 114 proteins fulfilled at least one of these criteria (FIGS. 16 and 17). Twelve of the proteins from the unpaired analysis were downregulated in both Early Post PE and Late Post PE compared with Pre PE (depicted in the intersection of the two lower circles of FIG. 5). Interestingly, some of the proteins were downregulated both in comparisons between Early Post PE and/or Late Post PE with Pre PE (lower circles of FIG. 5), and also in comparisons between Early Post PE and Post C and Late post PE and Late post C (lower circles of FIGS. 6 and 7, respectively). The overlapping proteins are depicted in FIG. 11 and are further elaborated in FIGS. 21 and 22. Also, a considerable proportion of the proteins were found to be decreased in PE samples and at the same time increased in control samples, these observations are further elaborated in FIGS. 23 and 24. The negative markers of PE are listed in FIG. 16, 25% of them were not identified in the condensate from the mechanical ventilator (FIG. 25). Bioinformatics analysis showed that also these proteins were related to a significantly higher extent than expected for a random set of proteins (FIGS. 12 and 37). Also in case of these proteins, a large part were extracellular proteins.

### Example 2: Pilot Study

Animals

Seven female Danish Landrace pigs of 60 kg were included. The experimental pigs were breed at a local specific pathogen free farmer and transferred to the research animal facility at Aarhus University for quarantine at least 7 days before the day of the experiment. The study protocol was approved by the Danish Animal Experiment Inspectorate (license number 2016-15-0201-00840), the experiments followed national and international guidelines concerning ethical care of experimental animals and Danish legislation on transport of livestock.

Anesthesia, Ventilation and Basic Monitoring

Anesthesia was induced with intravenous Etomidate (0.5 mg/kg, Hypnomidate®Janssen Parmaceutical, Belgium) and maintained after intubation with continuous intravenous infusion of Propofol (2 mg/kg/hour, Propolipid, Fresenius Kabi, Germany) and Fentanyl (5 μg/kg/hour, Hamlen Pharma, Germany). Pressure controlled volume gated ventilation (Datex-Ohmeda S/5 Avance) with non-humidified air, tidal volume of 8 ml/kg, respiratory rate of 16 breaths/minute and no positive end-expiratory pressure was initiated. End tidal CO₂ before induction of PE was maintained at 5.0 kPa by altering tidal volume and respiratory rate if necessary. Initially after intubation, the fraction of inspired O₂ was set to 1.0 for correction of hypoxemia during the procedure, after stabilization it was reduced to 0.3.

A rectal temperature of 38-39° C. was maintained. Peripheral saturation was measured on the tale or ear. At the end of the experiment, the pigs were euthanatized by intravenous injection of an overdose of Phenobarbital (67 mg/kg, Exagon®, vet, Richter Pharma, Austria).

Catheterizations and Embolus Formation

Intravascular catheters were placed guided by ultrasound in the left femoral artery (for continuous measurement of blood pressure and heart rate (HR) and drawing of arterial blood samples), the left external jugular vein (for continuous infusion of the anesthetics, drawing of blood for the emboli and venous blood samples). In total, 120 ml of autologous blood was drawn and distributed to four ¾″ Uncoated Extra Corporal Tubes and left for coagulation at room temperature for approximately three hours. Serum was discharged before the embolus was transferred to an uncoated tube containing isotone saline, which was then connected to a 26 French (F) Dry-Seal sheath placed in the right external jugular vein. Isotonic saline was used to gently flush the embolus through the 26 F Dry-Seal sheath to the right atrium, from where the embolus was transported to the pulmonary artery system by the circulation. Two emboli were administered for each pig with a one hour interval. In all seven pigs, the embolus lodged in the central pulmonary arteries, the exact position was evaluated by Magnetic Resonance Imaging (MRI) after each embolus. Cardiac output (CO) and dimensions of the right ventricle (RVD) were obtained by MRI. Mean pulmonary artery blood pressure (MPAP) were measured at baseline and after induction of each embolus by A Swan-Ganz catheter introduced through the 26 F Dry-Seal sheath. Invasive hemodynamic measures and functional measures were obtained 80 minutes after the first embolus was administered.

Collection and Handling of Exhaled Breath Condensate

Two series of EBC collection were carried out in each pig; the first sample (Pre EBC) was collected after approximately one hour of mechanical ventilation, after insertion of the catheters in the femoral artery and in the left external jugular vein, but before catheterization of the right external jugular vein. The 26 F Dry-Seal sheath was then placed, and the pig was transported to the MRI center for pulmonary angiography, functional measures and right heart catheterizations before, in between and after administering of the two emboli. Second round of EBC collection (Post EBC) was initiated approximately 2% hours after induction of the first pulmonary embolus, after the pig was transported back to the research facility. For pig number 5, 6 and 7 two EBC samples were collected Pre PE and two EBC samples were collected Post PE as we were concerned about having enough material for the subsequent analysis. Arterial blood gasses were drawn concurrently.

The EBC was collected from the expiratory limb of the mechanical ventilator by insertion of a polypropylene collection tube (RTubeVent, Respiratory Research Inc., Austin, Tex. USA) positioned inline just after the Y-connector (FIG. 2). An aluminum cooling sleeve surrounding the collection tube precooled to either −21° C. or −80° C. condensed the vapor of the exhaled breath. The condensing temperature was −21° C. in the first four experimental animals. To increase the condensation efficiency the condensing temperature in the last three experimental animals was reduced to −80° C. The cooling sleeve was insulated to reduce heating during the 15-20 minutes of EBC collection. Upon EBC collection the heat and moisture exchanger filter was disconnected. Immediately after collection, EBCs was stored in the collection tubes at −80° C. until further use. Volume of EBC was estimated either by pipetting or by weighing taking 1 gram as 1 ml. Samples were vacuum centrifuged and re-dissolved in 120 μl digestion buffer (0.5% SDC, 20 mM TEAB). The fluorescence (Excitation at 295 nm, Emission at 350 nm) was measured in microtiter trays using 100 μl. A standard curve was constructed from tryptophan. The protein concentration was estimated under the presumption that 1 g of protein corresponds to 0.0117 gram of tryptophan as it is the case for human and mouse protein samples.

Filter-Aided Sample Preparation with In-Solution Digestion and Phase Transfer (FASP-ISD-PT)

The sample was added to a Microcon 30K centrifugal filter device (Merck Milipore Ltd., Tullagreen, IRL) and centrifuged at 14,000 g for 10 min. One hundred μl of 50 mM iodoacetamide (IAA) was added, mixed at 600 rpm for 1 min. and incubated at room temperature for 20 min. in the dark. The filter was centrifuged and 100 μl digestion buffer was added two times each followed by centrifugation. Then 35 μl digestion buffer was added together with 0.5 μg of trypsin and left overnight at 37° C. in a wet chamber. Fifty μl digestion buffer was added and the filter unit centrifuged at 14,000 g for 10 min. with new collection tubes. Trifluoroacetic acid (TFA) was added to a final concentration of 0.5% (v/v) and an equal sample volume of ethyl acetate was added, shaken for 1 min. and centrifuged at maximum speed for 2 min. The lower phase was saved by removal of the upper phase. The extraction was repeated two times. The samples were dried in a vaccuum centrifuge and re-suspended in 0.1% (v/v) formic acid.

Nano Liquid Chromatography-Tandem Mass Spectrometry (nLC-MS/MS)

The peptide mixture was separated by nLC on an UltiMate 3000 (Thermo Scientific) coupled to an Orbitrap Fusion mass spectrometer (Thermo Scientific) through an EASY-Spray source (Thermo Scientific). A trap column (300 μm×5 mm, C18 PepMap100, 5 μm, 100 Å, Thermo Scientific) was used to load the sample and an analytical column (EASY-Spray Column, 500 mm×75 μm, PepMap RSCL, C18, 2 mm, 100 Å, Thermo Scientific) was used to separate peptides. The peptides were eluted with a flow of 300 nl/min using a 60 min. gradient by mixing buffer A (0.1% formic acid) with buffer B (80% acetonitrile, 20% water, 0.1% formic acid). The following amount of buffer B was used, 6% (0 min.), 16% (3 min.), 30% (30 min.), 60% (38 min.), 99% (40 min.), 99% (50 min.), 6% (51 min.), 6% (60 min.). The universal method setting was used for mass spectrometry detection with full Orbitrap scans (m/z 400-1500) at a resolution of 120,000. Automatic gain control (AGC) target of 4×10⁵ and a maximum injection time of 50 ms was used. The cycle time was 3 sec. The most intense precursors were selected with an intensity threshold of 5×10³ in top speed data dependent mode. MS² scans were performed in the linear ion trap in auto scan range mode with CID energy set at 35%, an AGC target of 2×10³ and a maximum injection time of 300 ms. The precursor ions with charge states 2-7 were isolated using the quadrupole with an isolation window of 1.6 m/z. Dynamic exclusion was set to 60 s.

Identification and Label-Free Quantification of Proteins

The raw data files were used to search the Uniprot databases, Sus scrofa and Homo sapiens, each downloaded on the 22 Feb. 2017, using MaxQuant (v1.5.5.1) for LFQ analysis. Fixed modification was carbamidomethyl (C). The false discovery rate was set to 1% for PSM, protein and site and the LFQ minimum ratio count was set at 1. MS/MS was required for LFQ comparisons. Unique and razor peptides, unmodified and modified with oxidation (M) or acetyl (protein N-terminal), were used for protein quantification with a minimum ratio count of 2. The match between runs function was activated. Reverse sequences were used for decoy search. Contaminant sequences were included. The results were entered into Perseus (v1.5.8.5) for further processing. Proteins were filtered by removing potential contaminants, reverse and proteins only identified by site. LFQ-values were log 2 transformed. Technical replicates were averaged by mean. The samples were then categorized as pre PE EBC or post PE EBC and also according to condensing temperature (−20° C. or −80° C.). Proteins were filtered by requiring razor+unique >1. The proteins were further filtered by requiring that each protein was identified in all EBC samples prior to PE and after PE.

Bioinformatics

After removal of poorly identified proteins from the dataset a bioinformatic analysis was conducted in GeneCodis3 to bring insights into cellular compartments of proteins identified in the EBCs. To ensure that all proteins were recognized in GeneCodis3 Homo sapiens was chosen as organism. Cellular compartments were chosen in GeneCodis3. In GeneCodis, a hypergeometric test was used and p-values were corrected using the false discovery rate method of Benjamini and Hochberg. Cellular compartments with a corrected p-value <0.05 were accepted as statistically significant.

Principal Component Analysis (PCA)

The PCA analysis was based on proteins identified in all the EBC samples in the dataset and the LFQ values were log 2 transformed before the analysis.

A PCA analysis was conducted using default settings in Perseus in order to analyze if the samples could be separated according to initial collection temperature.

Tracking of Extracellular Vesicles

EBC samples were analyzed for presence of extracellular vesicles isolated by ultracentrifugation at 20.000 g, re-suspending the pellet in phosphate buffer and sought quantified via Nano-particle tracking analysis.

Statistics Concerning Clinical Data

In analyses where clinical data, protein concentrations and EBC volumes were compared according to initial collection temperature, data were analyzed as independent samples by the Students t-test. The assumption of normality was checked by inspection of Q-Q plots, when violated, the Wilcoxon rank sum test was used. In a linear regression model, we estimated the difference in amount of protein per 100 liter of exhaled air as the difference in mean protein amount after linear adjustment for collection time. The volume of the exhaled air from which the EBC was obtained was calculated as the tidal volume multiplied by the respiratory frequency/minute multiplied by the collection time (minutes). Estimates are given as mean with associated 95% confidence intervals (CI) and p-values. The model was checked by plots of the residuals.

When the data were compared according to collection time (Pre PE vs. Post PE), data were analyzed as paired samples based on the t-test, assumptions of equal distributions and normality was checked by Bland-Altman plots and Q-Q plots of the differences, respectively. For pig number 5, 6 and 7, the protein concentrations in the EBC in the Pre PEa and Pre PEb EBC samples averaged by the mean and the EBC volume, the total amount of protein and the total amount of exhaled air during the EBC collection were used in the paired analysis. Estimates are given as mean±standard error of the mean (SEM). Statistical analysis of clinical data and descriptive statistics of protein amount, concentrations and EBC volumes were performed in STATA version 14.0 (Stata corporation, College Station, Tex., USA) software package.

Results

Clinical Data

Mean weight of the pigs was 58.9 kg. Mean room temperature in the research facility was 22.6° C. The pigs had significantly higher mean pulmonary artery pressure and heart rate after PE, but preserved cardiac output and mean arterial pressure. The end systolic volume of the right ventricle diameter was larger after PE and the plasma levels of troponin T were increased; in summary the porcine model corresponded to intermediate-high-risk PE. Arterial blood gas analysis showed higher PaCO₂ after PE. Due to hypoxemia during transport from the MRI center to the research facility two of the pigs were given FiO2 of 0.75-1.0 during collection of post EBS samples, tidal volume and respiratory rate was however unaltered. The transitory hypoxemia should not change the effect of condensing temperature on protein concentration of the EBC or the protein concentration in the EBC to such a degree that these animals ought to be excluded.

Volume of Exhaled Air, EBC, Protein Amount and Concentrations

The mean minute ventilation was 6.85±0.24 l/min with no differences according to collection temperature (p-value 0.79) or in the Pre EBC versus the Post EBC as the respiratory frequency and tidal volume was unaltered in each pig. The mean volume of EBC was 1216.0±176.7 μl, significantly higher when condensing temperature was −80° C. instead of −20° C. (1779±250 μl versus 707±123 μl, p<0.001). The mean protein concentration in the EBC was 6.01±0.88 ng/ml, not altered by the collection temperature (mean protein concentration 5.33±1.60 ng/μl in the EBC collected at −21° C. compared with 6.36±1.01 ng/μl in the EBC samples collected at −80° C., p-value 0.59). The mean protein amount was 6.52±1.04 μg per EBC sample with significant higher amounts when collected at −80° C. (9.91±1.45 μg) compared with EBCs collected at −21° C. (3.33±0.76 μg, p-value<0.001).

The mean concentration of protein in the exhaled air was 4.96 μg/100 liter exhaled air ±0.94. In the EBCs collected at −80° C., the protein amount per 100 liter of exhaled air was significantly higher compared with the EBCs collected at −21° C. (respectively 3.31±0.87 μg/100 l and 7.98±0.92 μg/100 l, p-value 0.02). Collection times were longer, however not significantly, for EBC samples collected at −80° C. (15.1±0.2 minutes for EBC collected at −21° C. versus 18.1±0.8 minutes for those collected at −80° C., p-value 0.13). Due to this, the EBCs collected at −80° C. were sampled from larger volumes of exhaled air (104.1 liters±4.6 for the EBC collected at −21° C. versus 123.75 liters±8.3 for the EBC collected at −80° C., p-value 0.05).

In a linear regression model we assessed the effect of collection time on the total amount of protein per 100 liter increase in total exhaled air; in the unadjusted model, it was predicted that an increase in the total volume of exhaled air by 100 liter during the collection of EBC would result in additionally 11.84 ug protein (95% CI 3.05 to 20.63, p-value 0.01). When adjusted for the collection time however, this difference disappeared (6.87 ug, 95% Cl-8.27.03 to 22.02, p-value 0.35). Hence, the difference in protein yield per 100 liter increase in exhaled air can to a large degree be attributed to longer collection periods, which was possible when initial collection temperature was lower.

The mean concentration of protein in the exhaled air did not differ in the Pre PE EBCs compared with the Post PE EBC (p-value 0.48), neither did the total protein amount (p-value 0.58) or the protein concentration in the EBC (p-value 0.26).

In conclusion, a collection temperature of −80° C. allowed sampling from larger volumes of exhaled air due to longer collection periods. Furthermore, the total protein yield was higher because the condensation was more efficient compared with a collection temperature of −21° C. No differences in total protein yield or concentrations in the EBC or exhaled air was observed after PE compared with before PE.

Label-Free Quantification and Bioinformatics

A total of 254 proteins were successfully identified in the EBC, average number of proteins per sample was 209 (range 166-234). Of these, 131 proteins were identified and quantified in all EBC samples. These 131 proteins were grouped according to their cellular localization using the bioinformatics tool GeneCodis3. A protein can be localized in more than one cellular compartment. Cellular compartments of the identified proteins included cytoplasm (73 proteins), cytosol (47 proteins), nucleus (40 proteins), extracellular region (31 proteins), plasma membrane (28 proteins), mitochondrion (18 proteins), intracellular (13 proteins), soluble fraction (12 proteins), cytoskeleton (11 proteins), extracellular space (11 proteins), melanosome (11 proteins) and protein complex (10 proteins).

Principal Component Analysis (PCA)

A PCA plot was made in order to test if the samples could be separated according to condensing temperature. The PCA plot revealed that samples did not group according to condensing temperature. Thus, the sample did not group into two well-defined groups based on initial collection temperature.

Extracellular Vesicles

Neither the Nano-sight nor the western blotting procedures showed presence of extracellular vesicles in the EBC.

### Example 3: Human Trials

All of the above examples, and methods used therein, are repeated in a human clinical trial with the exception of the mechanical ventilator, which is substituted for a mouthpiece that the participants breathe voluntarily into.

1. Use of an assay comprising exhaled breath condensate for detection of pulmonary embolism.

2. The assay according to claim 1, wherein said assay is an antibody-based detection assay.

3. The assay according to any of the preceding claims, wherein said antibody-based detection assay is an ELISA, a western blot or a protein immunoprecipitation assay.

4. The assay according to claim 1, wherein said assay is mass spectrometry, such as nano liquid chromatography-tandem mass spectrometry, such as high-performance liquid chromatography (HPLC).

5. The assay according to any of the preceding claims, wherein said assay further comprises the detection of one or more biomarkers.

6. The assay according to any of the preceding claims, wherein the one or more biomarkers are proteins.

7. The assay according to any of the preceding claims, wherein the one or more biomarkers are those having a p-value below 0.10 selected from FIGS. 15 and 16.

8. The assay according to any of the preceding claims, wherein the one or more biomarkers are those having a fold-change of at least 10 selected from FIGS. 15 and 16.

9. The assay according to any of the preceding claims, wherein the one or more biomarkers are Tropomyosin alpha-3 (TPM3), destrin (DSTN), protein S100-A11 (S100A11), arginase 1 (ARG1) and/or apolipoprotein D (APOD).

10. The assay according to any of the preceding claims, wherein the one or more biomarkers associated with pulmonary embolism are selected from the group set out in tables 1 and 2.

11. The assay according to any of the preceding claims, wherein the one or more of said markers are positively correlated with pulmonary embolism.

12. The assay according to claim 11, wherein the one or more markers are selected those set out in from table 1.

13. The assay according to any of the preceding claims, wherein the one or more markers are negatively correlated with pulmonary embolism.

14. The assay according to claim 13, wherein the one or more markers are selected from those set out in from table 2.

15. The assay according to any of the preceding claims, wherein the one or more biomarkers are intracellular proteins.

16. The assay according to claim 15, wherein the one or more biomarkers are intracellular proteins provided in FIG. 41.

17. The assay according to any of the preceding claims, wherein the one or more biomarkers are major plasma proteins and/or proteins associated with hemoptysis.

18. The assay according to claim 17, wherein the one or more biomarkers are the major plasma proteins and/or proteins associated with hemoptysis provided in FIG. 42.

19. The assay according to claim 18, wherein the one or more biomarkers are one or more major plasma proteins selected from a group consisting of: SERPINA1, C3, HP, TF, FN1, LTF, SERPINC1, PLG, A2M, FGA, FGB, FGG, KLKB, serpins superfamily, Immunoglobulin superfamily, ORM1 and ORM2.

20. The assay according to any of the preceding claims, wherein the one or more biomarkers are one or more proteins associated with coagulation.

21. The assay according to claim 20, wherein the one or more biomarkers are selected from a group consisting of: CDC42, ANXA5, SERPINA1, CLU, C3, SERPINC1 and SERPIN B2.

22. The assay according to any of the preceding claims, wherein the one or more biomarkers are one or more proteins associated with hemostasis and/or heme metabolism.

23. The assay according to any of the preceding claims, wherein the one or more biomarkers are hemoglobin scavenger proteins.

24. The assay according to any of claim 22 or 23, wherein the one or more biomarkers are selected from a group consisting of: HMOX, HPX, HP, ALB, APOA1, VCL, ACTB, CFL1 and TUBA1B.

25. The assay according to any of the preceding claims, wherein the one or more biomarkers are inflammatory proteins.

26. The assay according to claim 25, wherein the one or more biomarkers are one or more inflammatory proteins provided in FIG. 45.

27. The assay according to any of the preceding claims, wherein the one or more biomarkers are one or more proteins provided in FIG. 46.

28. The assay according to any of the preceding claims, wherein the one or more biomarkers are one or more proteins provided in FIG. 47.

29. The assay according to any of the preceding claims, wherein the one or more biomarkers are one or more proteins provided in FIG. 49.

30. The assay according to any of the preceding claims, wherein the one or more biomarkers are one or more proteins provided in FIG. 49A.

31. The assay according to any of the preceding claims, wherein the one or more biomarkers are one or more proteins provided in FIG. 49B.

32. The assay according to any of the preceding claims, wherein the one or more biomarkers are one or more proteins provided in FIG. 50.

33. A method for determining pulmonary embolism and/or increased risk thereof in a human being comprising
a. collecting a sample of exhalation air from said human being and
b. determining the presence or absence in said exhaled air of one or more biomarkers associated with pulmonary embolism or increased risk thereof.

34. The method according to claim 33, wherein said sample of exhalation air is an exhaled breath condensate sample.

35. The method according to any of claims 33-34, wherein said one or more biomarkers associated with pulmonary embolism or increased risk thereof are selected from the group set out in tables 1 and 2.

36. The method according to any of claims 33-35, wherein said one or more biomarkers are those having a p-value below 0.10 selected from FIGS. 15 and 16.

37. The method according to any of claims 33-36, wherein said one or more biomarkers are those having a fold-change of at least 10 selected from FIGS. 15 and 16.

38. The method according to any of claims 33-37, wherein said one or more biomarkers are Tropomyosin alpha-3 (TPM3), destrin (DSTN), protein S100-A11 (S100A11), arginase 1 (ARG1) and/or apolipoprotein D (APOD).

39. The method according to any of claims 33-38, wherein one or more of said markers are positively correlated with pulmonary embolism.

40. The method according to claim 39, wherein said one or more markers are selected those set out in from table 1.

41. The method according to any of claims 33-40, wherein one or more of said markers are negatively correlated with pulmonary embolism.

42. The method according to claim 41, wherein said one or more markers are selected from those set out in from table 2.

43. The method according to any of claims 33-42, wherein the level of said one or more biomarkers are determined.

44. The method according to any of claims 33-43, wherein the level of said one or more biomarkers is determined by antibody-based detection assay such as an ELISA, such as western blot, such as protein immunoprecipitation.

45. The method according to any of claims 33-44, wherein the level of said one or more biomarkers is determined by methods of protein detection such as mass spectrometry, such as nano liquid chromatography-tandem mass spectrometry, such as high-performance liquid chromatography (HPLC).

46. A kit comprising means for detecting at least one biomarker associated with pulmonary embolism or risk thereof.

47. The kit according to claim 46, said kit comprising an indicator strip comprising said means for detecting at least one biomarker associated with pulmonary embolism or risk thereof.
